(19) United States
(12) Patent Application Publication
(10) Pub. No.: US 2023/0054595 A1
(43) Pub. Date: Feb. 23, 2023

(54) NOVEL DRUGGABLE TARGETS FOR THE TREATMENT OF INFLAMMATORY DISEASES SUCH AS SYSTEMIC LUPUS ERYTHEMATOSUS (SLE) AND METHODS FOR DIAGNOSIS AND TREATMENT USING THE SAME

(71) Applicant: THE CHILDREN'S HOSPITAL OF PHILADELPHIA, Philadelphia, PA (US)

(72) Inventors: Struan Grant, Swarthmore, PA (US); Andrew Wells, Berwyn, PA (US); Chun Su, Secane, PA (US)

(73) Assignee: THE CHILDREN'S HOSPITAL OF PHILADELPHIA, Philadelphia, PA (US)

(21) Appl. No.: 17/787,433

(22) PCT Filed: Dec. 18, 2020
(86) PCT No.: PCT/US20/66246

§ 371 Date: Jun. 20, 2022

Related U.S. Application Data
(60) Provisional application No. 62/949,833, filed on Dec. 18, 2019.

(51) Int. Cl. C12Q 1/6883; C12N 15/113; A61P 37/00

(52) U.S. Cl. CPC C12Q 1/6883; C12N 15/1137; C12Y 207/11001; A61P 37/00

(57) ABSTRACT

Compositions and methods for the management and treatment of inflammatory disorders including SLE are disclosed.

### CROSS-REFERENCE TO RELATED APPLICATIONS

This application claims priority to U.S. Provisional Patent Application No. 62/949,833, filed on Dec. 18, 2019, which is incorporated herein by reference in its entirety.

### FIELD OF THE INVENTION

The present invention relates the fields of inflammatory disease and gene mapping. More specifically, the present invention provides compositions and methods for identifying new gene targets associated with inflammatory diseases such as SLE, new genes so identified, and agents useful for treatment and management of such diseases.

### BACKGROUND OF THE INVENTION

Several publications and patent documents are cited throughout the specification in order to describe the state of the art to which this invention pertains. Each of these citations is incorporated by reference herein as though set forth in full.

GWAS has been an important tool in understanding the genetic basis of complex, heritable metabolic, neurological, and inflammatory diseases. However, GWAS is typically powered to identify relatively large blocks of the genome containing dozens to hundreds of single nucleotide polymorphisms (SNP) in linkage disequilibrium (LD), any one of which could be responsible for the association of the entire locus with disease susceptibility. Moreover, ˜90% of GWAS-implicated SNP are intergenic or intronic, and do not affect the coding sequence of proteins. Therefore, the location of the GWAS signal per se does not identify the culprit gene(s). Examples of this are the FTO GWAS signal in obesity^{1,2}, and the TCF7L2 GWAS signal in type 2 diabetes³, in which each top variant resides in an intron of the local gene, but were shown instead to regulate expression of the distant genes IRX3/5 and ACSL5, respectively.

Systemic lupus erythematosus (SLE) is a complex inflammatory disease mediated by autoreactive antibodies that damage skin, joints, kidneys, brain and other tissues in children and adults⁴. An important inflammatory leukocyte required for the development of SLE is the follicular helper T cell (TFH). TFH differentiate from naïve CD4+ T cells in the lymph nodes, spleen, and tonsil, where they license B cells to produce high affinity protective or pathogenic antibodies^{5,6}. Given the central role for TFH in the regulation of humoral immune responses, genetic susceptibility to SLE is highly likely to manifest functionally in this immune cell population.

GWAS has associated over 60 loci with SLE susceptibility to date^{7,8}, but this represents thousands of SNP in LD that could potentially contribute to disease. Clearly, a need exists in the art to identify new and useful targets and therapeutics for treatment and management of inflammatory disorders such as SLE.

### SUMMARY OF THE INVENTION

In accordance with the present invention, a method for alleviating inflammatory disease symptoms in a patient in need thereof is disclosed. In one embodiment, a biological sample is obtained from said patient, wherein the sample comprises nucleic acids. An inflammatory disease GWAS causal variant in a gene which is indicative of the presence of, or increased risk for, inflammatory disease is then identified followed by treatment of said patient with an effective amount of at least one agent which targets said gene harboring said causal variant, thereby alleviating inflammatory disease symptoms. The inflammatory disease can include, without limitation systemic lupus erythematosus (SLE), arthritis, rheumatoid arthritis, psoriatic arthritis, juvenile idiopathic arthritis, multiple sclerosis, myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, scleroderma, systemic sclerosis, or allergic asthma. In certain embodiments, the inflammatory disease is SLE.

Table 1 provides information relating to proximal and sentinel SNPs, inferred GWAS genes and genes implicated in 3D epigenomics assays which can be used to advantage in the practice of the present invention. Table 2 provides genes implicated in inflammatory disease and suitable therapeutic targeting the listed genes. In certain embodiments, the gene is MINK1 and the agent is a MAP3/4K antagonist.

Also provided is a method for identifying at least one agent useful for the treatment of inflammatory disease comprising; providing a cell harboring at least one gene comprising an informative SNP for inflammatory disease in a cell type of interest and a cell which lacks said informative SNP; incubating said cells in the presence with an agent; and identifying agents which alter the function of said gene in cells harboring said SNP relative to those lacking said SNP. In certain embodiments, the cells are selected from tonsil follicular T helper cells, naïve CD4+ T cells, naïve CD8+ T cells, memory CD4+ T cells, memory CD8+ T cells, cytotoxic T lymphocytes, naïve B cells, germinal center B cells, Th1 cells, Th2 cells, Th17 cells, NK cells, dendritic cells, monocytes

In a preferred embodiment a method for treatment of SLE is provided comprising administration of an effective amount of a MAP3/4K antagonist, wherein said treatment alleviates SLE symptoms. In certain approaches the agent is PF06260933. In each case, the agents used for treatment can also be combined with agents conventionally used to treat inflammatory disease, such as a steroid.

In other embodiments, a method for treatment of SLE comprising administration of an effective amount of a pharmacological modulator of HIPK1 is disclosed wherein the treatment alleviates SLE symptoms.

The invention also provides transgenic mice harboring insertions or deletions in one or more genes listed in Table 1. In certain embodiments, the mice are knockout mice for HIPK1 or MINK1. In other embodiment, the mice harbor immune disorder associated mutations identified in patients having one or more disorders described herein below. In preferred embodiments, the patient harbors a mutation associated with the SLE phenotype.

Also provided are EBV transformed cell lines harboring a mutations in at least one gene listed in Table 1. In certain embodiments, the mutations are in the HIPK1 or MINK1 genes, preferably obtained from patient DNA.

### BRIEF DESCRIPTION OF THE DRAWINGS

FIG. 1A-1C. TFH and naive T cells show comparable genomic accessibility. Overall log 2 fold changes in reference OCR accessibility (CPM) in TFH compared to naive T cells represented by density plot (FIG. 1A) or distribution plot (FIG. 1B). FIG. 1C. The accessibility signal was normalized by the counts per million method and mean p values across three replicates were used for comparison between TFH and naive T cells.

FIG. 2A-2B. ATAC-seq analysis of open chromatin landscapes in naïve and follicular helper T cells from human tonsil. FIG. 2A. Quantitative differences between naive and follicular helper T cell open chromatin landscapes. A total of 91,222 OCR were used as reference for differential analysis of genome accessibility. The number of statistically up- (green) or down- (red) regulated OCR in TFH compared to naïve is shown as a Venn diagram and also plotted as a function of log 2 fold change. FIG. 2B. GSEA enrichment analysis of genes with differential promoter accessibility at promoter regions. The log 2 fold change in expression between naïve and follicular helper T cells was used to generate the pre-ranked list for GSEA.

FIG. 3A-3B. Canonical pathway enrichment for genes with accessible SLE SNPs in their promoters. The log 2FDR (blue) and gene ratios (red) for the top 10 enriched Ingenuity canonical pathways is shown for TFH (FIG. 3A) and naive (FIG. 3B) cells.

FIG. 4A-4C. Genes harboring accessible SLE variants in naïve and follicular helper T cells. FIG. 4A. Comparison of accessible SLE SNPs between TFH and naïve tonsillar T cells. FIG. 4B. Comparison of the expression levels of genes with accessible SLE SNPs in their promoters in TFH vs. all genes or a random sample of genes with no accessible SLE SNPs in their promoters. FIG. 4C. Ingenuity disease network for the genes with accessible SLE variants at promoters. The color gradient represents the log 2 fold change IN expression in TFH compared to naïve T cells.

FIG. 5A-5D. High-resolution, fragment-based Capture-C analysis of promoter connectomes in naïve and follicular helper T cells. FIG. 5A. Cartoon depicting the approaches for 1 DpnII fragment promoter interaction analysis, 4 DpnII fragment promoter interaction analysis, and promoter-OCR interaction analysis. FIG. 5B. The relationship between the number of interactions per gene promoter and expression of the corresponding gene is shown. Gene expression was binned into the lowest 20th, 20-40th, 40-60th, 60-80th and >80th percentiles. Lower and upper boxplot hinges correspond to the first and third quartiles, and outliers were defined as >1.5*IQR from the hinge. WashU browser depictions of fragment-based promoter interactions and ATAC-seq accessibility at the IL21 (FIG. 5C) and IFNG (FIG. 5D) loci in TFH (red) and naive T cells (blue). Color gradients represent the CHiCAGO scores.

FIG. 6A-6C. Enrichment of chromatin signatures at promoter interacting regions. FIG. 6A. PIR enrichment for genomic features compared with distance-matched random regions in naive T cells. Error bars show SD across 100 draws of non-significant interactions. FIG. 6B. Feature enrichment at promoter-interacting OCR (iOCR) compared to a random sample of non-promoter-interacting OCR in naive T cells. FIG. 6C. Enrichment of iOCR within chromHMM-defined chromatin states and TSS neighborhood in naive T cells. ChromHMM 15-state models defined on the basis of 5 histone modifications (H3K4me1, H3K4me3, H3K27me3, H3K27ac and H3K36me3) are shown in the middle panel, with blue color intensity representing the probability of observing the mark in each state. The heatmap to the left of the emission parameters displays the overlap fold enrichment for iOCR in promoters (priOCR) and non-promoter iOCR (npriOCR), while the heatmap to the right shows the fold enrichment for each state within 2 kb around a set of TSS. Blue color intensity represents fold enrichment.

FIG. 7A-7C. Enrichment of chromatin signatures at promoter interacting regions in TFH cells. FIG. 7A. PIR enrichment for genomic features compared with distance-matched random regions in TFH cells. Error bars show SD across 100 draws of non-significant interactions. FIG. 7B. Feature enrichment of promoter-interacting OCR (iOCR) compared to a random sample of non-promoter-interacting OCR in TFH. FIG. 7C. Enrichment of iOCR within chromHMM-defined chromatin states and TSS neighborhood in TFH. Roadmap Epigenomics 15-state models (middle panel) were defined on the basis of 5 histone modifications (H3K4me1, H3K4me3, H3K27me3, H3K27ac and H3K36me3). Blue color intensity represents the probability of observing the mark in the state. The heatmap to the left of the emission parameters displays the overlap fold enrichment for different categories of iOCR, while the heatmap to the right shows the fold enrichment for each state within 2 kb around a set of TSS. Blue color intensity represents fold enrichment.

FIG. 8A-8D. Analysis of promoter-open chromatin connectomes in naïve and follicular helper T cells. FIG. 8A. Comparison of promoter-OCR interactions between TFH and naïve T cells. Relationship between the number of promoter-OCR interactions at a gene and its corresponding expression level in naive T cells (FIG. 8B) and TFH (FIG. 8C) are depicted with genes with recognized functions in naive and TFH labeled. FIG. 8D. Promoter-OCR interactions and ATAC-seq accessibility at the CD28, CTLA4 and ICOS loci in TFH (red) and naive T cells (blue).

FIG. 9. Distribution of promoter-interacting OCR per gene in naïve T and TFH cells. The number of promoter-interacting OCR per gene is plotted for both naïve T (red) and TFH (blue) cells.

FIG. 10A-10B. Immune networks enriched among SLE SNP connectome implicated gene sets. The top 3 merged immune networks in naïve (FIG. 10A) and TFH (FIG. 10B) are depicted. Red color intensity represents the number of interactions detected per promoter for each gene in the network.

FIG. 11A-11D. SLE variant-to-gene mapping through integration of GWAS and promoter-open chromatin connectomes in follicular helper T cells. Four categories of accessible SLE SNP-promoter interactions were detected. FIG. 11A. Accessible SLE SNP uniquely interacts with the nearest promoter (8.5%). An example is rs527619, which physically interacts only with the nearest gene STAT4. FIG. 11B. Accessible SLE SNP interacts with the nearest promoter and at least one distant promoter (29%). An example is rs112677036, which physically interacts with IKZF3 and the distant ERBB2 and PGAP3 genes. FIG. 11C. Accessible SLE SNP ‘skips’ the nearest promoter to interact exclusively with on or more distant promoters (62.5%). Examples are (c) rs34631447, which skips LPP to physically interact with BCL6, and (FIG. 11D) rs527619 and rs71041848, which interact with the distant CXCR5 gene instead of the nearest gene, TREH.

FIG. 12A-12B. Interaction of open SLE variants with genes encoding nuclear proteins targeted by autoantibodies in SLE patients. FIG. 12A. The accessible SNP rs3117582 at the promoter of APOM physically interacts with the LSM2 promoter. FIG. 12B. The accessible SNP rs7769961 at the SNRPC promoter physically interacts with the UHRF1BP1 promoter.

FIG. 13A-13C. Ontology and pathway analysis of genes implicated through integration of GWAS and promoter-open chromatin connectomes in follicular helper T cells from human tonsil. FIG. 13A. Enrichment of the top 25 canonical pathways (FIG. 13A) and 3 disease networks (FIG. 13B) among genes implicated through promoter-open chromatin connectomes in TFH. FIG. 13C. Regulatory hierarchical network from SLE-connectome-implicated genes. Color gradients in FIG. 13B and FIG. 13C represent log 2 expression changes between TFH and naïve T cells, with green indicating down-regulation and red indicating up-regulation in TFH. Blue nodes in FIG. 13C represent regulatory hubs for genes with no SLE-OCR connectome detected.

FIG. 14. Comparison of SLE SNP-gene associations obtained by promoter-open chromatin connectomes vs. eQTL studies. Comparison of sentinel SNP-gene pairs implicated by the promoter-open chromatin connectomes in this study vs. sentinel SNP-gene pairs statistically associated in two SLE eQTL studies7,29. SNP-gene pairs shared by each group are detailed.

FIG. 15A-15F. CRISPR/CAS9-based editing of accessible genomic regions containing SLE GWAS proxy SNP in Jurkat T cells. FIG. 15A, UCSC genome browser track displaying intergenic TFH OCR near the TREH gene harboring the rs527619 and rs71041848 proxies to the rs4639966 SLE sentinel SNP that interacts with CXCR5 (chr11:118,563,185-118,563,321). Eight sgRNAs flanking the OCR and Sanger sequencing identifying several deletions present within the OCR are depicted. FIG. 15B, Electrophoresis gel analysis of PCR amplified regions encompassing the targeted region showing two distinct deletions present at 500 bp and 350 bp. FIG. 15C, Genomic region surrounding the TFH OCR containing the rs4385425 SNP proxy to the sentinel SLE SNP rs11185603 (chr7:50307234-50307447) that interacts with IKZF1, showing sgRNAs and deletions detected in targeted Jurkat lines. Note that this OCR was called by HOMER, but not by MACS2. FIG. 15D, Electrophoresis gel analysis detects three different deletions at 900 bp, 400 bp and 350 bp. FIG. 15E, Intronic OCR (chr3:188,472,234-188,472,390) in the LPP locus harboring the rs34631447 and rs79044630 SNPs proxy to sentinel rs6762714 SLE SNP and found connected to BCL6. This region was targeted with five total sgRNAs surrounding the OCR and Sanger sequencing showed two distinct deletions. FIG. 15F, Electrophoresis gel analysis detects 1200 bp and 821 bp deletions. All experiments were performed in three biological replicates.

FIG. 16A-16C. CRISPR/CAS9-based deletion of accessible, promoter-connected genomic regions harboring SLE variants influences TFH gene expression. FIG. 16A, CRISPR-CAS9 targeting of the 136 bp OCR near the TREH gene harboring the rs527619 and rs71041848 SLE proxy SNPs and captured interacting with the CXCR5 promoter leads to increased CXCR5 expression (blue histogram) by Jurkat cells compared to cells transduced with a CTRL-gRNA+CAS9 (pink histogram). FIG. 16B, CRISPR-CAS9 targeting of the 213 bp OCR containing the rs4385425 SLE proxy and captured interacting with the IKZF1 promoter leads to increased IKZF1 (Ikaros) expression (blue histogram) by Jurkat cells compared to cells transduced with a CTRL-gRNA+CAS9 (pink histogram). FIG. 16C, CRISPR-CAS9 targeting of the LPP SLE proxy SNPs rs34631447 and rs79044630 captured interacting with the BCL6 promoter abrogates IFNγ-induced BCL-6 expression (orange histogram) compared to cells transduced with a CTRL-gRNA+CAS9 (blue histogram). The red histogram shows BCL-6 expression by unstimulated Jurkat cells. Bar graphs in a-c depict the mean fluorescence intensity (upper panels) and percent positive cells (lower panels) for CXCR5, Ikaros, and BCL-6 in control gRNA-transduced vs. targeted cells. All data are representative of three independent experiments. See FIG. 15 for design and validation of CRISPR/CAS9-mediated deletion and mutation.

FIG. 17A-17G. SLE variant-to-gene mapping implicates novel drug targets for modulation of TFH function. FIG. 17A. The interactomes of SLE proxy SNPs rs11552449, rs71368520, and rs71368521 implicate HIPK1 and MINK1. FIG. 17B. Purified naïve CD4+ T cells cultured under TFH-skewing conditions show increased expression of PD-1 and CXCR5, as well as BCL-6 (FIG. 17C). FIG. 17D. HIPK1 mRNA expression by in vitro-differentiated TFH cells transduced with scrambled Lenti-shRNA or Lenti-HIPK-1 shRNA. FIG. 17E. shRNA-mediated knock-down of HIPK1 inhibits IL-21 secretion by TFH cells. FIG. 17F. A HIPK inhibitory drug causes dose-dependent inhibition of IL-21 production by TFH cells. FIG. 17G. A MINK inhibitory drug causes dose-dependent inhibition of IL-21 production by TFH cells. All data are representative of 3-4 independent experiments.

FIG. 18A-18E. Promoter-variant connectome-guided targeting of novel kinases for modulation of primary human TFH function. FIG. 18A, Lentiviral delivery of B2M shRNA and HIPK1 shRNA into in vitro differentiated TFH as assessed by GFP fluorescence by flow cytometry. FIG. 18B, Assessment of shRNA-mediated knock-down of B2M and HIPK1 in TFH by flow cytometry and qRT-PCR. Red histograms are TFH transduced with scrambled control shRNA, and blue histograms depict TFH transduced with specific B2M (left panel) or HIPK1 (right panel) shRNA. FIG. 18C, Effect of HIPK inhibitory drug treatment on TFH differentiation in vitro as measured by co-induction of PD-1 and CXCR5. FIG. 18D, The HIPK inhibitory drug A64 does not affect IL-2 secretion by TFH cells as measured by ELISA. FIG. 18E, A MINK inhibitory drug inhibits IL-2 secretion by activated T cells with an ED50 of ˜50 nM. All data are representative of 3-4 replicate experiments.

FIG. 19. 3D epigenomic map of promoter-Capture-C, ATAC-seq, H3K27ac, and H3K4me1 in the BCL6-LPP region in naïve (blue) and TFH cells (red).

### DETAILED DESCRIPTION

Genome-wide association studies (GWAS) have statistically implicated hundreds of loci in the susceptibility to human disease, but the majority have failed to identify the causal variants or the effector genes. As an alternative, physicochemical approach to detecting functional variants and linking them to target genes, we generated comprehensive, high resolution maps of systemic lupus erythematosus (SLE) variant accessibility and gene connectivity in the context of the three-dimensional chromosomal architecture of human tonsillar follicular helper T cells (TFH) from three healthy individuals. Spatial epigenomic maps of this cell type, which is required for the production of anti-nuclear antibodies characteristic of SLE, identified over 400 potentially functional variants across 63 GWAS-implicated SLE loci. Twenty percent of these variants were located in open promoters of highly-expressed TFH genes, while 80% reside in non-promoter genomic regions that are connected in 3D to genes that likewise tend to be highly expressed in this immune cell type. Importantly, we find that 90% of SLE variants exhibit spatial proximity to genes that are not nearby in the 1D sequence of the genome, and over 60% of variants ‘skip’ the nearest gene to physically interact only with the promoters of distant genes. Gene ontology confirmed that genes in spatial proximity to SLE variants reside in highly SLE-relevant networks, including accessible SLE variants that loop 200-1000 kb to interact with the promoters of the canonical TFH genes BCL6 and CXCR5. CRISPR-Cas9 genome editing confirmed that these variants reside in novel, distal regulatory elements required for normal BCL6 and CXCR5 expression by T cells. Furthermore, SLE-associated SNP-promoter interactomes implicated a set of novel genes with no known role in TFH or SLE disease biology, including the homeobox-interacting protein kinase HIPK1 and the Ste kinase homolog MINK1. Targeting these kinases in primary human TFH cells inhibited production of IL-21, a requisite cytokine for production of class-switched antibodies by B cells. This 3D-variant-to-gene mapping approach gives mechanistic insight into the disease-associated regulatory architecture of the human genome.

### Definitions

The phrase “inflammatory disease” refers to a variety of disorders, which include for example, systemic lupus erythematosus (SLE), arthritis, rheumatoid arthritis, psoriatic arthritis, juvenile idiopathic arthritis, multiple sclerosis, myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, scleroderma, systemic sclerosis, or allergic asthma.

In certain embodiments, compositions are disclosed herein which are useful for the preparation of a medicinal product for treating and/or preventing skin lesions associated with autoimmune and/or inflammatory diseases in a human subject, and to a method for preventing and/or treating skin lesions associated with autoimmune and/or inflammatory diseases comprising the administration of the same to a human subject

In some aspects, the target will be skin lesions associated with autoimmune and/or inflammatory diseases selected from, in particular, lupus erythematosus, scleroderma, psoriasis, cutaneous vasculitis, vascular purpura, autoimmune bullous dermatoses (in particular bullous pemphigoid, cicatricial pemphigoid, linear IgA dermatosis, dermatitis herpetiformis, epidermolysis bullosa acquisita, pemphigus and variants thereof), dermatitis, (in particular atopic dermatitis, seborrheic dermatitis, stasis dermatitis), dermatomyositis, erythema nodosum, pyoderma gangrenosum, eczema (in particular eczema atopic, contact eczema, dyshidrotic eczema), lichen planus, lichen sclerosus et atrophicus, and alopecia areata. Preferentially the skin lesions associated with autoimmune or inflammatory diseases are skin lesions associated with lupus.

As used herein, the term “lupus” is equivalent to the term “lupus erythematosus” and comprises cutaneous lupus erythematosus (CLE) and disseminated lupus erythematosus (DLE) or systemic lupus erythematosus (SLE). CLE is a particularly polymorphic affection traditionally divided into three groups: chronic CLE, which includes discoid lupus, tumid lupus, lupus pernio and lupus profundus (or panniculitis); subacute CLE (SCLE); and acute CLE (ACLE).

The term “diagnosis” refers to a relative probability that a disease (e.g. an autoimmune, inflammatory autoimmune, cancer, infectious, immune, or other disease) is present in the subject. The term “prognosis” refers to a relative probability that a certain future outcome may occur in the subject with respect to a disease state. For example, in the present context, prognosis can refer to the likelihood that an individual will develop a disease (e.g. an autoimmune, inflammatory autoimmune, cancer, infectious, immune, or other disease), or the likely severity of the disease (e.g., extent of pathological effect and duration of disease). The terms are not intended to be absolute, as will be appreciated by any one of skill in the field of medical diagnostics.

As used herein, the term “treatment” or “treating” encompasses prophylaxis and/or therapy. Accordingly, the compositions and methods of the present invention are not limited to therapeutic applications and can be used in prophylaxis ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

Generally, an “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound. As used herein, the term “pharmaceutically effective amount” refers to a dose or quantity that causes improvement in at least one objective or subjective inflammation associated symptom, but not limited to: a reduction in flare ups, joint stiffness, a reduction in neurological symptoms, reduction in or lessening of skin lesion formation, and improvement in kidney function.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, skin cells, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods disclosed herein. The biopsy technique applied will depend on the tissue type to be evaluated (i.e., prostate, lymph node, liver, bone marrow, blood cell, joint tissue, synovial tissue, synoviocytes, fibroblast-like synoviocytes, macrophage-like synoviocytes, immune cells, hematopoietic cells, fibroblasts, macrophages, T cells, etc.), the size and type of a tumor (i.e., solid or suspended (i.e., blood or ascites)), among other factors. Representative biopsy techniques include excisional biopsy, incisional biopsy, needle biopsy, surgical biopsy, and bone marrow biopsy. Biopsy techniques are discussed, for example, in Harrison's Principles of Internal Medicine, Kasper, et al., eds., 16th ed., 2005, Chapter 70, and throughout Part V.

The phrase “Capture C” refers to a method for profiling chromosomal interactions involving targeted regions of interest, such as gene promoters, globally and at high resolution.

A “single nucleotide polymorphism (SNP)” refers to a change in which a single base in the DNA differs from the usual base at that position. These single base changes are called SNPs or “snips.” Millions of SNP's have been cataloged in the human genome. Some SNPs such as that which causes sickle cell are responsible for disease. Other SNPs are normal variations in the genome.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. In embodiments, antibodies or fragments of antibodies may be derived from different organisms, including humans, mice, rats, hamsters, camels, etc. Antibodies disclosed herein may include antibodies that have been modified or mutated at one or more amino acid positions to improve or modulate a desired function of the antibody (e.g. glycosylation, expression, antigen recognition, effector functions, antigen binding, specificity, etc.).

An “inhibitory nucleic acid” is a nucleic acid (e.g. DNA, RNA, polymer of nucleotide analogs) that is capable of binding to a target nucleic acid and reducing transcription of the target nucleic acid (e.g. mRNA from DNA) or reducing the translation of the target nucleic acid (e.g., mRNA) or altering transcript splicing (e.g. single stranded morpholino oligo). A “morpholino oligo” may be alternatively referred to as a “morpholino nucleic acid” and refers to morpholine-containing nucleic acid nucleic acids commonly known in the art (e.g. phosphoramidate morpholinio oligo or a “PMO”). See Marcos, P., Biochemical and Biophysical Research Communications 358 (2007) 521-527. In embodiments, the “inhibitory nucleic acid” is a nucleic acid that is capable of binding (e.g. hybridizing) to a target nucleic acid (e.g. an mRNA translatable into a protein) and reducing translation of the target nucleic acid. The target nucleic acid is or includes one or more target nucleic acid sequences to which the inhibitory nucleic acid binds (e.g. hybridizes). Thus, an inhibitory nucleic acid typically is or includes a sequence (also referred to herein as an “antisense nucleic acid sequence”) that is capable of hybridizing to at least a portion of a target nucleic acid at a target nucleic acid sequence. An example of an inhibitory nucleic acid is an antisense nucleic acid.

An “antisense nucleic acid” is a nucleic acid (e.g. DNA, RNA or analogs thereof) that is at least partially complementary to at least a portion of a specific target nucleic acid (e.g. a target nucleic acid sequence), such as an mRNA molecule (e.g. a target mRNA molecule) (see, e.g., Weintraub, Scientific American, 262:40 (1990)), for example antisense, siRNA, shRNA, shmiRNA, miRNA (microRNA). Thus, antisense nucleic acids are capable of hybridizing to (e.g. selectively hybridizing to) a target nucleic acid (e.g. target mRNA). In embodiments, the antisense nucleic acid hybridizes to the target nucleic acid sequence (e.g. mRNA) under stringent hybridization conditions. In embodiments, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and -anomeric sugar-phosphate, backbone-modified nucleotides. Another example of an inhibitory nucleic acid is siRNA or RNAi (including their derivatives or pre-cursors, such as nucleotide analogs). Further examples include shRNA, miRNA, shmiRNA, or certain of their derivatives or pre-cursors. In embodiments, the inhibitory nucleic acid is single stranded. In embodiments, the inhibitory nucleic acid is double stranded.

In embodiments, an antisense nucleic acid is a morpholino oligo. In embodiments, a morpholino oligo is a single stranded antisense nucleic acid, as is known in the art. In embodiments, a morpholino oligo decreases protein expression of a target, reduces translation of the target mRNA, reduces translation initiation of the target mRNA, or modifies transcript splicing. In embodiments, the morpholino oligo is conjugated to a cell permeable moiety (e.g. peptide). Antisense nucleic acids may be single or double stranded nucleic acids.

In the cell, the antisense nucleic acids may hybridize to the target mRNA, forming a double-stranded molecule. The antisense nucleic acids, interfere with the translation of the mRNA, since the cell will not translate a mRNA that is double-stranded. The use of antisense methods to inhibit the in vitro translation of genes is well known in the art (Marcus-Sakura, Anal. Biochem., 172:289, (1988)). Antisense molecules which bind directly to the DNA may be used.

The compositions of the invention, including without limitation, small molecules, kinase inhibitors and inhibitory nucleic acids can be delivered to the subject using any appropriate means known in the art, including by injection, inhalation, or oral ingestion. Another suitable delivery system is a colloidal dispersion system such as, for example, macromolecule complexes, nanocapsules, microspheres, beads, and lipid-based systems including oil-in-water emulsions, micelles, mixed micelles, and liposomes. An example of a colloidal system is a liposome. Liposomes are artificial membrane vesicles which are useful as delivery vehicles in vitro and in vivo. Nucleic acids, including RNA and DNA within liposomes and be delivered to cells in a biologically active form (Fraley, et al., Trends Biochem. Sci., 6:77, 1981). Liposomes can be targeted to specific cell types or tissues using any means known in the art Inhibitory nucleic acids (e.g. antisense nucleic acids, morpholino oligos) may be delivered to a cell using cell permeable delivery systems (e.g. cell permeable peptides). In embodiments, inhibitory nucleic acids are delivered to specific cells or tissues using viral vectors or viruses.

An “siRNA” refers to a nucleic acid that forms a double stranded RNA, which double stranded RNA has the ability to reduce or inhibit expression of a gene or target gene when the siRNA is present (e.g. expressed) in the same cell as the gene or target gene. The siRNA is typically about 5 to about 100 nucleotides in length, more typically about 10 to about 50 nucleotides in length, more typically about 15 to about 30 nucleotides in length, most typically about 20-30 base nucleotides, or about 20-25 or about 24-29 nucleotides in length, e.g., 20, 21, 22, 23, 24, 25, 26, 27, 28, 29, or 30 nucleotides in length. siRNA molecules and methods of generating them are described in, e.g., Bass, 2001, Nature, 411, 428-429; Elbashir et al., 2001, Nature, 411, 494-498; WO 00/44895; WO 01/36646; WO 99/32619; WO 00/01846; WO 01/29058; WO 99/07409; and WO 00/44914. A DNA molecule that transcribes dsRNA or siRNA (for instance, as a hairpin duplex) also provides RNAi. DNA molecules for transcribing dsRNA are disclosed in U.S. Pat. No. 6,573,099, and in U.S. Patent Application Publication Nos. 2002/0160393 and 2003/0027783, and Tuschl and Borkhardt, Molecular Interventions, 2:158 (2002).

The siRNA can be administered directly or siRNA expression vectors can be used to induce RNAi that have different design criteria. A vector can have inserted two inverted repeats separated by a short spacer sequence and ending with a string of T's which serve to terminate transcription.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the functional and novel characteristics of the sequence.

“Target nucleic acid” as used herein refers to a previously defined region of a nucleic acid present in a complex nucleic acid mixture wherein the defined wild-type region contains at least one known nucleotide variation which may or may not be associated with inflammatory disease. The nucleic acid molecule may be isolated from a natural source by cDNA cloning or subtractive hybridization or synthesized manually. The nucleic acid molecule may be synthesized manually by the triester synthetic method or by using an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated.

The term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus, if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any inflammatory disease specific marker gene or nucleic acid, but does not hybridize to other nucleotides. Also, polynucleotides which “specifically hybridizes” may hybridize only to an inflammatory disease specific marker, such an inflammatory disease-specific marker shown in the Appendix contained herein. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tₘ=81.5° C.+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the T is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washing in 0.1×SSC and 0.5% SDS at 65° C. for 15 minutes.

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension, product.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the inflammatory disease specific marker nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the inflammatory disease specific marker gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the proteins encoded by the inflammatory disease associated nucleic acids described herein. Agents are evaluated for potential biological activity by inclusion in screening assays described hereinbelow.

### Kits and Articles of Manufacture

Any of the aforementioned products can be incorporated into a kit which may contain a inflammatory disease-associated specific marker polynucleotide or one or more such markers immobilized on a Gene Chip, an oligonucleotide, a polypeptide, a peptide, an antibody, a label, marker, or reporter, a pharmaceutically acceptable carrier, a physiologically acceptable carrier, instructions for use, a container, a vessel for administration, an assay substrate, or any combination thereof

### Methods of Using Inflammatory Disease-Associated Specific Markers for Development of Therapeutic Agents

Since the genes identified herein have been associated with the etiology of inflammatory disease, methods for identifying agents that modulate the activity of the genes and their encoded products the identified SNPs should result in the generation of efficacious therapeutic agents for the treatment of a variety of disorders associated with this condition.

As can be seen from the data provided in the Tables, several chromosomes contain regions which provide suitable targets for the rational design of therapeutic agents which modulate their activity. Small peptide molecules corresponding to these regions may be used to advantage in the design of therapeutic agents which effectively modulate the activity of the encoded proteins.

Molecular modeling should facilitate the identification of specific organic molecules with capacity to bind to the active site of the proteins encoded by the inflammatory disease associated nucleic acids based on conformation or key amino acid residues required for function. A combinatorial chemistry approach will be used to identify molecules with greatest activity and then iterations of these molecules will be developed for further cycles of screening.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as described above) which have a nonfunctional or altered inflammatory disease associated gene. These host cell lines or cells are defective at the polypeptide level. The host cell lines or cells are grown in the presence of drug compound. The rate of cellular metabolism of the host cells is measured to determine if the compound is capable of regulating the cellular metabolism in the defective cells. Host cells contemplated for use in the present invention include but are not limited to bacterial cells, fungal cells, insect cells, mammalian cells, and plant cells. The inflammatory disease-associated DNA molecules may be introduced singly into such host cells or in combination to assess the phenotype of cells conferred by such expression. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

A wide variety of expression vectors are available that can be modified to express the novel DNA sequences of this invention. The specific vectors exemplified herein are merely illustrative, and are not intended to limit the scope of the invention. Expression methods are described by Sambrook et al. Molecular Cloning: A Laboratory Manual or Current Protocols in Molecular Biology 16.3-17.44 (1989). Expression methods in Saccharomyces are also described in Current Protocols in Molecular Biology (1989).

Suitable vectors for use in practicing the invention include prokaryotic vectors such as the pNH vectors (Stratagene Inc., 11099 N. Torrey Pines Rd., La Jolla, Calif. 92037), pET vectors (Novogen Inc., 565 Science Dr., Madison, Wis. 53711) and the pGEX vectors (Pharmacia LKB Biotechnology Inc., Piscataway, N.J. 08854). Examples of eukaryotic vectors useful in practicing the present invention include the vectors pRc/CMV, pRc/RSV, and pREP (Invitrogen, 11588 Sorrento Valley Rd., San Diego, Calif. 92121); pcDNA3.1/V5&His (Invitrogen); baculovirus vectors such as pVL1392, pVL1393, or pAC360 (Invitrogen); and yeast vectors such as YRP17, YIPS, and YEP24 (New England Biolabs, Beverly, Mass.), as well as pRS403 and pRS413 Stratagene Inc.); Picchia vectors such as pHIL-D1 (Phillips Petroleum Co., Bartlesville, Okla. 74004); retroviral vectors such as PLNCX and pLPCX (Clontech); and adenoviral and adeno-associated viral vectors.

Promoters for use in expression vectors of this invention include promoters that are operable in prokaryotic or eukaryotic cells. Promoters that are operable in prokaryotic cells include lactose (lac) control elements, bacteriophage lambda (pL) control elements, arabinose control elements, tryptophan (trp) control elements, bacteriophage T7 control elements, and hybrids thereof. Promoters that are operable in eukaryotic cells include Epstein Barr virus promoters, adenovirus promoters, SV40 promoters, Rous Sarcoma Virus promoters, cytomegalovirus (CMV) promoters, baculovirus promoters such as AcMNPV polyhedrin promoter, Picchia promoters such as the alcohol oxidase promoter, and Saccharomyces promoters such as the gal4 inducible promoter and the PGK constitutive promoter, as well as neuronal-specific platelet-derived growth factor promoter (PDGF), the Thy-1 promoter, the hamster and mouse Prion promoter (MoPrP), and the Glial fibrillar acidic protein (GFAP) for the expression of transgenes in glial cells.

In addition, a vector of this invention may contain any one of a number of various markers facilitating the selection of a transformed host cell. Such markers include genes associated with temperature sensitivity, drug resistance, or enzymes associated with phenotypic characteristics of the host organisms.

Host cells expressing the inflammatory disease-associated nucleic acids and proteins of the present invention or functional fragments thereof provide a system in which to screen potential compounds or agents for the ability to modulate the development of inflammatory disease, particularly SLE. Thus, in one embodiment, the nucleic acid molecules of the invention may be used to create recombinant cell lines for use in assays to identify agents which modulate aspects of cellular metabolism associated with immune cell signaling associated with inflammatory disease. Also provided herein are methods to screen for compounds capable of modulating the function of proteins encoded by the inflammatory disease associated nucleic acids described herein.

Another approach entails the use of phage display libraries engineered to express fragment of the polypeptides encoded by the inflammatory disease associated nucleic acids on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays. Such compound libraries are commercially available from a number of companies including but not limited to Maybridge Chemical Co., (Trevillet, Cornwall, UK), Comgenex (Princeton, N.J.), Microsour (New Milford, Conn.) Aldrich (Milwaukee, Wis.) Akos Consulting and Solutions GmbH (Basel, Switzerland), Ambinter (Paris, France), Asinex (Moscow, Russia) Aurora (Graz, Austria), BioFocus DPI (Switzerland), Bionet (Camelford, UK), Chembridge (San Diego, Calif.), Chem Div (San Diego, Calif.). The skilled person is aware of other sources and can readily purchase the same. Once therapeutically efficacious compounds are identified in the screening assays described herein, they can be formulated into pharmaceutical compositions and utilized for the treatment of inflammatory disease such as SLE.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacore.

Thus, one may design drugs which have, e.g., improved polypeptide activity or stability or which act as inhibitors, agonists, antagonists, etc. of polypeptide activity. By virtue of the availability of inflammatory disease associated nucleic acid sequences described herein, sufficient amounts of the encoded polypeptide may be made available to perform such analytical studies as x-ray crystallography. In addition, the knowledge of the protein sequence provided herein will guide those employing computer modeling techniques in place of, or in addition to x-ray crystallography.

In another embodiment, the availability of inflammatory disease-associated nucleic acids enables the production of strains of laboratory mice carrying the inflammatory disease-associated nucleic acids of the invention. Transgenic mice expressing the inflammatory disease-associated nucleic acids of the invention provide a model system in which to examine the role of the protein encoded by the nucleic acid (with or without a sentinel SNP) in the development and progression towards inflammatory disease. Methods of introducing transgenes in laboratory mice are known to those of skill in the art. Three common methods include: 1. integration of retroviral vectors encoding the foreign gene of interest into an early embryo; 2. injection of DNA into the pronucleus of a newly fertilized egg; and 3. the incorporation of genetically manipulated embryonic stem cells into an early embryo. Production of the transgenic mice described above will facilitate the molecular elucidation of the role that a target protein plays in various cellular metabolic and regulatory processes associated with aberrant inflammation. Such mice provide an in vivo screening tool to study putative therapeutic drugs in a whole animal model and are encompassed by the present invention.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The alteration of genetic information may be foreign to the species of animal to which the recipient belongs, or foreign only to the particular individual recipient, or may be genetic information already possessed by the recipient. In the last case, the altered or introduced gene may be expressed differently than the native gene. Such altered or foreign genetic information would encompass the introduction of inflammatory disease-associated nucleotide sequences and expression of proteins encoded thereby.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use isolated inflammatory disease-associated genes as insertional cassettes to selectively inactivate a wild-type gene in totipotent ES cells (such as those described above) and then generate transgenic mice. The use of gene-targeted ES cells in the generation of gene-targeted transgenic mice was described, and is reviewed elsewhere (Frohman et al., (1989) Cell 56:145-147; Bradley et al., (1992) Bio/Technology 10:534-539).

Techniques are available to inactivate or alter any genetic region to a mutation desired by using targeted homologous recombination to insert specific changes into chromosomal alleles. However, in comparison with homologous extrachromosomal recombination, which occurs at a frequency approaching 100%, homologous plasmid-chromosome recombination was originally reported to only be detected at frequencies between 10⁻⁶ and 10⁻⁸. Nonhomologous plasmid-chromosome interactions are more frequent occurring at levels 10⁵-fold to 10²-fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodou-racil, (FIAU). By this counter selection, the number of homologous recombinants in the surviving transformants can be increased. Utilizing inflammatory disease-associated SNP containing nucleic acid as a targeted insertional cassette provides means to detect a successful insertion as visualized, for example, by acquisition of immunoreactivity to an antibody immunologically specific for the polypeptide encoded by inflammatory disease-associated nucleic acid and, therefore, facilitates screening/selection of ES cells with the desired genotype.

As used herein, a knock-in animal is one in which the endogenous murine gene, for example, has been replaced with human inflammatory disease-associated gene of the invention.

Such knock-in animals provide an ideal model system for studying the development of inflammatory disease.

As used herein, the expression of an inflammatory disease-associated nucleic acid, fragment thereof, or an inflammatory disease-associated fusion protein can be targeted in a “tissue specific manner” or “cell type specific manner” using a vector in which nucleic acid sequences encoding all or a portion of inflammatory disease-associated nucleic acid are operably linked to regulatory sequences (e.g., promoters and/or enhancers) that direct expression of the encoded protein in a particular tissue or cell type. Such regulatory elements may be used to advantage for both in vitro and in vivo applications. Promoters for directing tissue specific proteins are well known in the art and described herein. The nucleic acid sequence encoding the inflammatory disease-associated sequence of the invention may be operably linked to a variety of different promoter sequences for expression in transgenic animals. Such promoters include, but are not limited to a platelet-derived growth factor B gene promoter, described in U.S. Pat. No. 5,811,633; a brain specific dystrophin promoter, described in U.S. Pat. No. 5,849,999; a Thy-1 promoter; a PGK promoter; a CMV promoter; a neuronal-specific platelet-derived growth factor B gene promoter; FOXP3 promoter for expression specifically in regulatory T cells and Glial fibrillar acidic protein (GFAP) promoter for the expression of transgenes in glial cells.

In certain embodiments, a conditional HIPK1 knock out mouse can be constructed to assess the impact of deletion of HIPK1 in specific immune cell types on immune responses to foreign and self antigens. MINK1 knock out mice can also be generated.

Methods of use for the transgenic mice of the invention are also provided herein. Transgenic mice into which a nucleic acid containing the inflammatory disease-associated nucleic acid, or its encoded protein have been introduced are useful, for example, to develop screening methods to screen therapeutic agents to identify those capable of modulating the development of inflammatory disease.

### Pharmaceuticals and Peptide Therapies

The elucidation of the role played by the inflammatory disease associated nucleic acids described herein in inflammation facilitates the development of pharmaceutical compositions useful for treatment and diagnosis of inflammatory disease. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

The following materials and methods are provided to facilitate the practice of the present invention.

Purification of Naïve and Follicular Helper T Cells from Human Tonsil

Fresh tonsils were obtained from immune-competent children (n=10) undergoing tonsillectomy to address airway obstruction or a history of recurrent tonsillitis. The mean age of donors was 5.7 years (range 2-16 years) and 50% were male. Tonsillar mononuclear cells were isolated from tissues by mechanical disruption (tonsils were minced and pressed through a 70 μM cell screen) followed by Ficoll-Paque centrifugation. CD19 positive cells were removed (StemCell) and CD4⁺ T cells were enriched with magnetic beads (Biolegend) prior to sorting naïve T cells (CD4⁺CD45RO⁻) and T follicular helper cells (CD4⁺CD45RO⁺CD25^{lo}CXCR5^{hi}PD1^{hi}) on a MoFlo Astrios EQ (Beckman Coulter).

### Cell Fixation

We used standard methods for cell fixation {Chesi:2019}. Briefly, 10⁷ TFH or naïve CD4+ T cells were suspended in 10 mL RPMI+10% FBS, followed by additional 270 uL of 37% formaldehyde and incubation for 10 min at RT on a platform rocker. The fixation reaction was quenched by the addition of 1.5 mL cold 1M glycine (4° C.). The fixed cells were centrifuged at 1000 rpm for 5 min at 4° C. and supernatants were removed. The cell pellets were washed in 10 ml cold PBS (4° C.) followed by centrifugation as above. Cell pellets were resuspended in 5 ml cold lysis buffer (10 mM Tris pH8, 10 mM NaCl, 0.2% NP-40 (Igepal) supplemented with a protease inhibitor cocktail). Resuspended cell pellets were incubated for 20 minutes on ice, centrifuged at 1800 rpm, and lysis buffer was removed. Cell pellets were resuspended in 1 mL of fresh lysis buffer, transferred to 1.5 mL Eppendorf tubes, and snap frozen in ethanol/dry ice or liquid nitrogen. Frozen cell pellets were stored at −80° C. for 3C library generation.

### 3C Library Generation

The generation of 3C libraries was performed as previously described {Chesi:2019}. For each library, 10⁷ fixed cells were thawed at 37° C., followed by centrifugation at RT for 5 mins at 14,000 rpm. The cell pellet was resuspended in 1 mL of dH₂O supplemented with 5 μL 200× protease inhibitor cocktail, incubated on ice for 10 mins, then centrifuged. Cell pellets were resuspended to a total volume of 650 μL in dH2O. 50 μL of cell suspension was set aside for pre-digestion QC, and the remaining sample was divided into 3 tubes. Both pre-digestion control and samples were undergo pre-digestion incubation in a Thermomixer (BenchMark) with added 0.3% SDS, 1×NEB DpnII Restriction Buffer, and dH₂O for 1 hr at 37° C. with shaking at 1,000 rpm. Addition 1.7% of Triton X-100 was added to each tube, continue shaking for another hour. After pre-digestion incubation, 10 μl of DpnII (NEB) (50 U/μL) was added to each sample tube only, and continue shaking along with pre-digestion control until the end of the day. An additional 10 of DpnII was added to each digestion reaction and digested overnight. The next day, a further 10 μL DpnII was added and continue shaking for another few hours. 100 uL of each digestion reaction was then removed and pooled to a new 1.5 mL tube and set aside for digestion efficiency QC. The remaining samples were heat inactivated incubated at 1000 rpm in a MultiTherm for 20 min at 65° C. to inactivate the DpnII, and cooled on ice for 20 additional minutes. Digested samples were ligated with 8 uL of T4 DNA ligase (HC ThermoFisher, 30 U/μL) and 1× ligase buffer at 1,000 rpm overnight at 16° C. in a BenchMark MultiTherm. The next day, an additional 2 μL of T4 DNA ligase was spiked into each sample and incubated for another few hours. The ligated samples were then de-crosslinked overnight at 65° C. with Proteinase K (20 mg/mL, Denville Scientific) along with pre-digestion and digestion control. The following morning, both controls and ligated samples were incubated for 30 min at 37° C. with RNase A (Millipore), followed by phenol/chloroform extraction, ethanol precipitation at −20° C., the 3C libraries were centrifuged at 3000 rpm for 45 min at 4° C. to pellet the samples. The controls were centrifuged at 14,000 rpm. The pellets were resuspended in 70% ethanol and centrifuged as described above. The pellets of 3C libraries and controls were resuspended in 300 uL and 20 μL dH₂O, respectively, and stored at −20° C. Sample concentrations were measured by Qubit. Digestion and ligation efficiencies were assessed by gel electrophoresis on a 0.9% agarose gel and also by quantitative PCR (SYBR green, Thermo Fisher).

### Promoter-Capture-C Design

The promoter-Capture-C approach described herein was designed to leverage the four-cutter restriction enzyme DpnII in order to give high resolution restriction fragments of a median of ˜250 bp {Chesi:2019}. This approach also allows for scalable resolution through in silico fragment concatenation (not shown). Custom capture baits were designed using Agilent SureSelect RNA probes targeting both ends of the DpnII restriction fragments containing promoters for coding mRNA, non-coding RNA, antisense RNA, snRNA, miRNA, snoRNA, and lincRNA transcripts (UCSC lincRNA transcripts and sno/miRNA under GRCh37/hg19 assembly) totaling 36,691 RNA baited fragments through the genome {Chesi:2019}. In this study, the capture library was re-annotated under gencodeV19 at both 1-fragment and 4-fragment resolution, and is successful in capturing 89% of all coding genes and 57% of noncoding RNA gene types. The missing coding genes could not be targeted due to duplication or highly repetitive DNA sequences in their promoter regions.

### Promoter-Capture-C Assay

Isolated DNA from 3C libraries was quantified using a Qubit fluorometer (Life technologies), and 10 μg of each library was sheared in dH₂O using a QSonica Q800R to an average fragment size of 350 bp. QSonica settings used were 60% amplitude, 30 s on, 30 s off, 2 min intervals, for a total of 5 intervals at 4° C. After shearing, DNA was purified using AMPureXP beads (Agencourt). DNA size was assessed on a Bioanalyzer 2100 using a DNA 1000 Chip (Agilent) and DNA concentration was checked via Qubit. SureSelect XT library prep kits (Agilent) were used to repair DNA ends and for adaptor ligation following the manufacturer protocol. Excess adaptors were removed using AMPureXP beads. Size and concentration were checked by Bioanalyzer using a DNA 1000 Chip and by Qubit fluorometer before hybridization. 1 μg of adaptor-ligated library was used as input for the SureSelect XT capture kit using manufacturer protocol and our custom-designed 41K promoter Capture-C library. The quantity and quality of the captured library was assessed by Bioanalyzer using a high sensitivity DNA Chip and by Qubit fluorometer. SureSelect XT libraries were then paired-end sequenced on 8 lanes of Illumina Hiseq 4000 platform (100 bp read length).

### ATAC-seq Library Generation

50,000 to 100,000 sorted tonsillar naive or follicular helper T cells were centrifuged at 550 g for 5 min at 4° C. The cell pellet was washed with cold PBS and resuspended in 50 μL cold lysis buffer (10 mM Tris-HCl, pH 7.4, 10 mM NaCl, 3 mM MgCl₂, 0.1% IGEPAL CA-630) and immediately centrifuged at 550 g for 10 min at 4° C. Nuclei were resuspended in the Nextera transposition reaction mix (25 ul 2×TD Buffer, 2.5 μL Nextera Tn5 transposase (Illumina Cat #FC-121-1030), and 22.5 ul nuclease free H₂O) on ice, then incubated for 45 min at 37° C. The tagmented DNA was then purified using the Qiagen MinElute kit eluted with 10.5 μL Elution Buffer (EB). 10 μl purified tagmented DNA was PCR amplified using Nextera primers for 12 cycles to generate each library. PCR reaction was subsequently cleaned up using 1.5×AMPureXP beads (Agencourt), and concentration was measured by Qubit. Library was then paired-end sequenced on the Illumina HiSeq 4000 platform (100 bp read length).

### ATAC-seq Analysis

TFH and naïve ATAC-seq peaks were called using the ENCODE ATAC-seq pipeline on the world wide web at www.encodeproject.org/atac-seq/. Briefly, pair-end reads from three biological replicates for each cell type were aligned to hg19 genome using bowtie2, and duplicate reads were removed from the alignment. Narrow peaks were called independently for each replicate using macs2 (-p 0.01 --nomodel --shift -75 --extsize 150 -B --SPMR --keep-dup all --call-summits) and ENCODE blacklist regions (ENCSR636HFF) were removed from peaks in individual replicates. Peaks from all replicates were merged by bedtools (v2.25.0) within each cell type and the merged peaks present in less than two biological replicates were removed from further analysis. Finally, ATAC-seq peaks from both cell types were merged to obtain reference open chromatin regions. To determine whether an OCR is present in TFH and/or naïve cells, we first intersected peaks identified from individual replicates in each cell type with reference OCRs. If any peaks from at least one replicate overlapped with a given reference OCR, we consider that region is open in the originating cell type. Quantitative comparisons of TFH and naïve open chromatin landscapes were performed by evaluating read count differences against the reference OCR set. De-duplicated read counts for OCR were calculated for each library and normalized against background (10K bins of genome) using the R package csaw (v 1.8.1). OCR peaks with less than 1.5 CPM (4.5-7.5 reads) support at top 3 libraries were removed from further differential analysis. Differential analysis was performed independently using edgeR (v 3.16.5) and limmaVoom (v 3.30.13). Differential OCR between cell types were called if FDR<0.05 and absolute log 2 fold change >1 in both methods.

### Promoter-Focused Capture-C Analysis

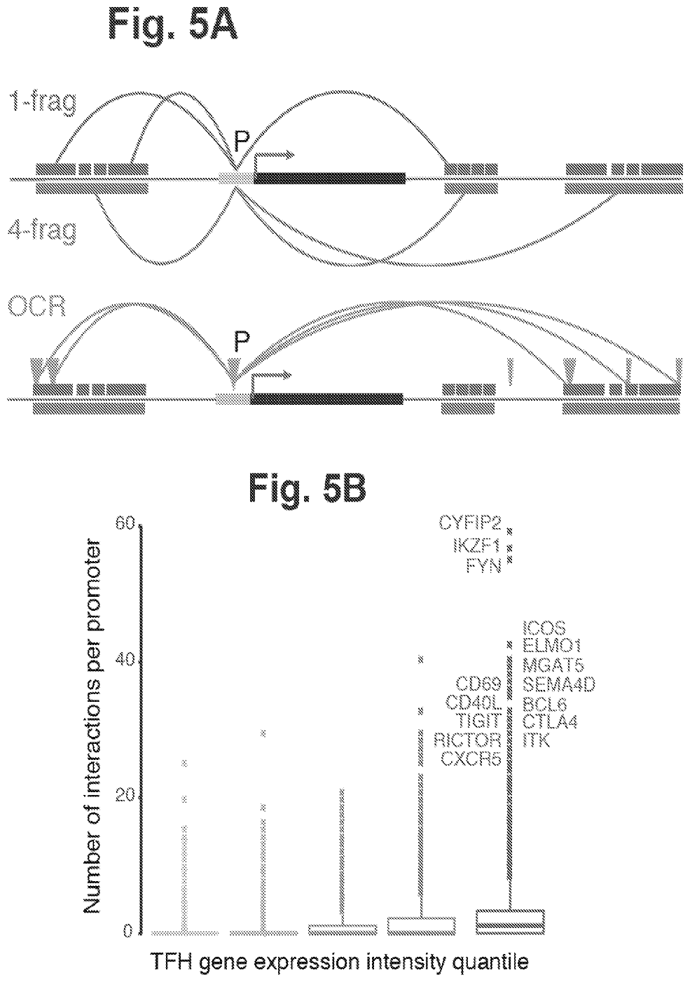

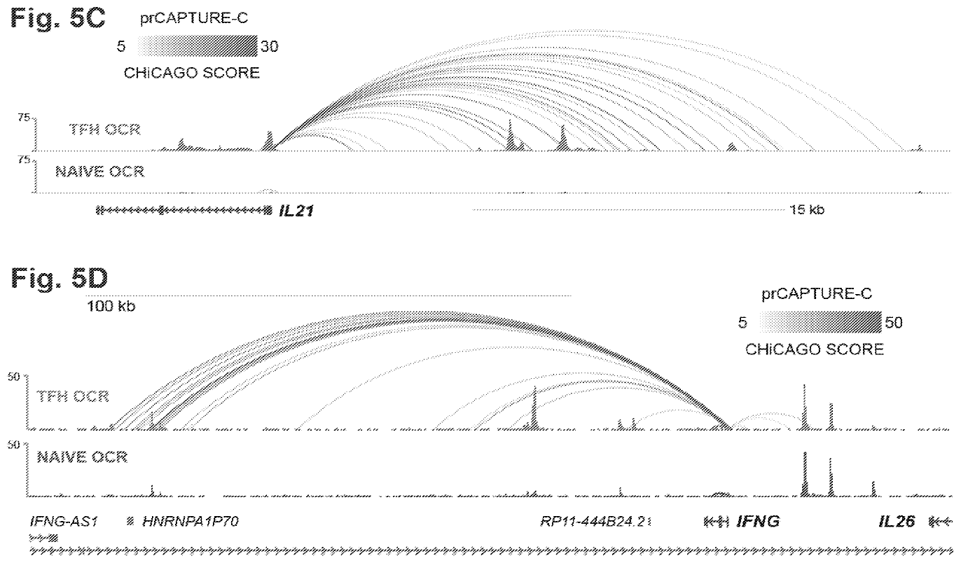

Paired-end reads from three biological replicates for naïve and follicular helper T cells were pre-processed using the HICUP pipeline (v0.5.9) {Wingett:2015}, with bowtie2 as aligner and hg19 as the reference genome. We were able to detect non-hybrid reads from all targeted promoters, validating the success of the promoter capture procedure. Significant promoter interactions at 1-DpnII fragment resolution were called using CHiCAGO (v1.1.8) {Cairns:2016} with default parameters except for binsize set to 2500. Significant interactions at 4-DpnII fragment resolution were also called using CHiCAGO with artificial .baitmap and .rmap files in which DpnII fragments were concatenated in silico into 4 consecutive fragments using default parameters except for removeAdjacent set to False. The significant interactions (CHiCAGO score >5) from both 1-fragment and 4-fragment resolutions were exported in .ibed format and merged into a single file using custom a PERL script to remove redundant interactions and to keep the max CHiCAGO score for each interaction. Open chromatin interaction landscapes were established by projecting significant DpnII fragment interactions at merged 1- and 4-fragment resolutions to reference OCR (FIG. 5A). First, DpnII fragments involved in significant interactions (both “bait” and “other end”) were intersected with reference OCR using bedtools (v2.25.0). Interactions between bait and other end OCR pairs were called independently for each cell type if their overlapped fragments interacted at either resolution and if both OCR were called as “open” in the corresponding cell type. OCR involved in promoter interactions (iOCR) were classified as promoter OCR (prOCR) or regulatory OCR (nonprOCR) by comparing their genomic locations to pre-defined promoter regions (−1500 bp˜500 bp of TSS) of transcripts in GENCODE V19 and UCSC noncoding RNA described above. If the OCR that were overlapped with bait fragments failed mapping to gene promoters, the OCR interactions were removed. OCR pair interactions were combined from both cell types to obtain the reference open chromatin promoter-captured interaction landscapes.

### Microarray Analysis of Gene Expression

RNA from two biological naïve tonsillar CD4+ T cell replicates and four biological tonsillar TFH replicates were hybridized to Affymetrix Human Clarion S arrays at the CHOP Nucleic Acid and Protein Core. Data were pre-processed (RMA normalization), and analyzed for differential expression (DE) using Transcriptome Analysis Console v 4.0 with a false discovery rate (FDR) threshold of 0.05 and a fold-change (FC) threshold of 2. Lists of differentially expressed genes were generated and ranked by log 2 fold change. The log 2 fold change of the genes with significantly differential accessibility at promoter regions were compared to the pre-ranked gene expression data for GSEA enrichment analysis.

### Gene Set Enrichment and Ingenuity Pathway Analysis

Histone mark and CTCF ChIP-seq datasets for naïve and follicular helper T cells were obtained from public resources¹⁹⁻²¹ and compared to promoter-interacting fragments or promoter-interacting OCR. Enrichment of promoter-interacted fragments (PIR) for histone marks and CTCF regions was determined independently in each cell type using the function peakEnrichment4Features( ) in the CHiCAGO package, and feature enrichment at promoter-interacting OCR were compared to enrichment at non-promoter-interacting OCR using the feature enrichment R package LOLA (v1.4.0)⁴⁴. Fisher's exact tests were performed and odd ratios were plotted for significant enrichment (pvalue<10⁻⁶) using ggplot2. The chromatin states of promoter-interacting OCR were also determined using ChromHMM (v1.17) on binarized bed file of histone marks ChIP-seq peaks with 15 states for naïve T cells and 6 states for TFH cells. The annotation of chromatin states was manually added with the reference to epigenome roadmap project²⁰. Ingenuity pathway analysis (IPA, QIAGEN) was used for all the pathway analysis. The top significantly enriched canonical pathways were plotted using ggplot2 and networks with relevant genes were directly exported from IPA.

### CRISPR/CAS9 Genome Editing

CRISPR guide RNAs (sgRNA) targeting rs34631447, rs79044630, rs527619, rs71041848, and rs4385425 were designed using http://crispr.tefor.net and cloned into lentiCRISPRv2-puro or lentiCRISPRv2-mCherry (Feng Zhang, Addgene plasmid #52961; http://n2t.net/addgene:52961; RRID:Addgene 52961) by golden gate ligation using the BsmB1 restriction enzyme (NEB). 293T cells were transfected in DMEM using Lipofectamine 2000 (Invitrogen) with 6 ug PsPAX2 and 3.5 ug PmD2.G packaging plasmids and 10 ug empty lentiCRISPRv2 or 10 ug sgRNA-encoding lentiCRISPRv2. Viral supernatants were collected after 48 hrs for transduction into Jurkat leukemic T cells maintained in RPMI 1640 with 10% fetal bovine serum, L-glutamine, 2-mercaptoethanol, and penicillin/streptomycin. Cells were seeded in a 24 well plate at 0.5×10⁶ in 0.5 mL of media per well, and 1 mL of viral supernatant with 8 ug/mL of polybrene was added to each well. Spin-fection was performed for 90 min. at 2500 rpm and 25° C., and transduced cells were equilibrated at 37° C. for 6 hrs. For rs34631447, rs79044630, and rs4385425, 1.2 ml of media was removed and replaced with 1 ml of fresh media containing 1 ug of puromycin for 7 days of selection before use in experiments. Cells transduced with sgRNAs targeting rs527619 and rs71041848 were sorted based on mCherry on a FACS Jazz (BD Biosciences). Mutations were analyzed by PCR coupled with Sanger sequencing at the CHOP Nucleic Acids and Protein Core. The following primers were used for PCR:

BLC6-
F:
(SEQ ID NO: 1)
CTCTGTGGTTGTGGGCAAGGC
R:
(SEQ ID NO: 2)
CAGGTGGCGAATCAGGACAGG,
CXCR5-
F:
(SEQ ID NO: 3)
GTCCCTGGTGATGGAAACTCAGGC-
R:
(SEQ ID NO: 2):
GCAGTGGCCTCCCTTACACAGG (SEQ ID NO: 4),
IKZF1-
F:
(SEQ ID NO: 5:
CCTTCTCCATGCCCAGGTGACTC-
R:
(SEQ ID NO: 6)
GGCCTCAGCTAGGCAAACCAGAG.

Measurement of BCL-6 expression in targeted Jurkat lines was assessed by flow cytometry using anti-human APC-BCL-6 (Biolegend) after treatment with human recombinant IFNγ (5 ng/mL, R&D Systems) overnight and stimulation with PMA (30 ng/mL) and ionomycin (1 Sigma-Aldrich) for 4-6 hrs. Expression of Ikaros and CXCR5 by targeted Jurkat lines was also assessed by flow cytometry using anti-human APC-CXCR5 (Biolegend) and anti-human PE-Ikaros (BD Biosciences). Fixation, permeabilization and intracellular staining for Ikaros and BCL-6 was performed using the Transcription Factor Buffer Set (BD Pharmingen). Cells were analyzed on a CytoFLEX flow cytometer (Beckman Coulter).
Lentiviral shRNA-Based Gene Targeting

A lentiviral shRNA-based approach was employed to silence the expression of HIPK1 as well as B2M as a positive control. The lenti-shRNA vectors pGFP-C-shRNA-Lenti-Hipk1, pGFP-C-shRNA-Lenti-B2M and pGFP-C-scrambled were purchased from Origene. The packaging vectors PmD2G and PsPAX.2 were obtained from Addgene. Exponentially growing 293T cells were split and seeded at 8×10⁶ cells in 100 mm dishes in RPMI 1640 medium at 37° C. The following day, cells were transfected in antibiotic- and serum-free medium with lenti shRNA plus packaging vector DNA prepared in a complex with Lipofectamine 2000. After 6 hrs of transfection, medium was replaced with complete serum containing RPMI medium and cells were cultured at 37° C. for 2 days. Human primary CD4+ T cells from healthy donors were obtained from the University of Pennsylvania Human Immunology Core and stimulated overnight with human anti-CD3- and anti-CD28-coated microbeads. Cells were harvested, de-beaded, washed with warm RPMI medium, and aliquots of 10⁶ activated CD4+ T cells were infected with 1 ml of viral supernatant collected from lenti-shRNA transfected 293T cell cultures. Polybrene was added to the viral supernatant at 8 ug/ml, cells were spin-fected at 2500 rpm for 1.5 hrs, cultured at 37° C. for 6 hrs, and restimulated with anti-CD3 and anti-CD28 beads, Activin A (100 ng/ml), IL-12 (5 ng/ml), and anti-IL-2 (2 ug/ml) to induce in vitro TFH differentiation {Locci;2016}. After 4 days of differentiation, transduced cells were FACS-sorted based on GFP expression, and expression of B2M, BCL-6, CXCR5 and PD-1 was measured by flow cytometry. In addition, sorted GFP+ in vitro TFH cells were restimulated with plate-bound human anti-CD3 and anti-CD28 (1 μg/ml each) in flat bottom 96 well plates, and supernatants were collected at the indicated timepoints for assessment of IL-21 secretion by ELISA. RNA was extracted from sorted GFP+TFH cells using an RNeasy micro kit (Qiagen), treated with DNase, and 500 ng of total RNA was reverse-transcribed using iScript cDNA synthesis kit (Bio-Rad). qRT-PCR quantification of HIPK-1, B2M and 18s rRNA transcripts was performed using Amplitaq Gold SYBR Master mix (ABI) on Applied Biosystems step one plus real-time thermocycler. Specific mRNA levels were determined as ratio of total 18s rRNA. The following primer sequences were used for qRT PCR:

| | HIPK-1-F: |
|---|---|
| (SEQ ID NO: 7) | |
| | CAGTCAGGAGTTCTCACGCA, |
| | HIPK-1-R: |
| (SEQ ID NO: 8) | |
| | TGGCTACTTGAGGGTGGAGA, |
| | B2M-F: |
| (SEQ ID NO: 9) | |
| | GCCGTGTGAACCATGTGACT, |
| | B2M-R: |
| (SEQ ID NO: 10) | |
| | CATCCAATCCAAATGCGGCA, |
| | hu 18S-F: |
| (SEQ ID NO: 11) | |
| | CCTTTAACGAGGATCCATTGGA, |
| | hu 18S-R: |
| (SEQ ID NO: 12) | |
| | CGCTATTGGAGCTGGAATTACC. |

### Pharmacologic Inhibitors

The HIPK kinase family inhibitor A64 trifluoroacetate was purchased from Sigma, and the MAP4K2 inhibitor PF06260933, which also inhibits MINK1 and TNIK, was purchased from TOCRIS. Human primary CD4+ T cells were cultured under TFH condition for 5 days in the presence of the indicated concentrations of each inhibitor (150 nM to 2500 nM for A64, 3.7 nM to 100 nM for PF06260933). In addition, anti-CD3- and anti-CD28-stimulated human CD4+ T cells (non-TFH) were cultured in the presence of inhibitors. After 5 days of primary culture, cells were harvested and 10⁶ cells were restimulated with plate-bound human anti-CD3+ anti-CD28 (1 ug/ml each) in the presence of inhibitors. Culture supernatants were collected at the indicated timepoints for measurement of IL-2 and IL-21 by ELISA (eBioscience).

### Data Availability

Our data are available from ArrayExpress (https://www.ebi.ac.uk/arrayexpress/) with accession numbers E-MTAB-6621 (promoter-Capture-C), E-MTAB-6617 (ATAC-seq), and E-MTAB-6637 (expression microarray) respectively.

The following examples are provided to illustrate certain embodiments of the invention. They are not intended to limit the invention in any way.

### EXAMPLES

### Comparative Open Chromatin Landscapes of Naïve CD4+ T Cells and TFH from Human Tonsil

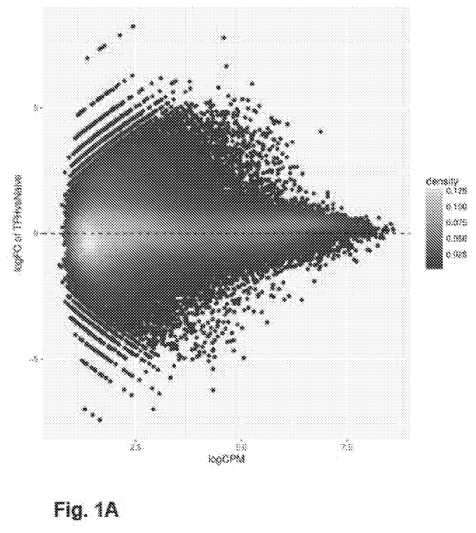

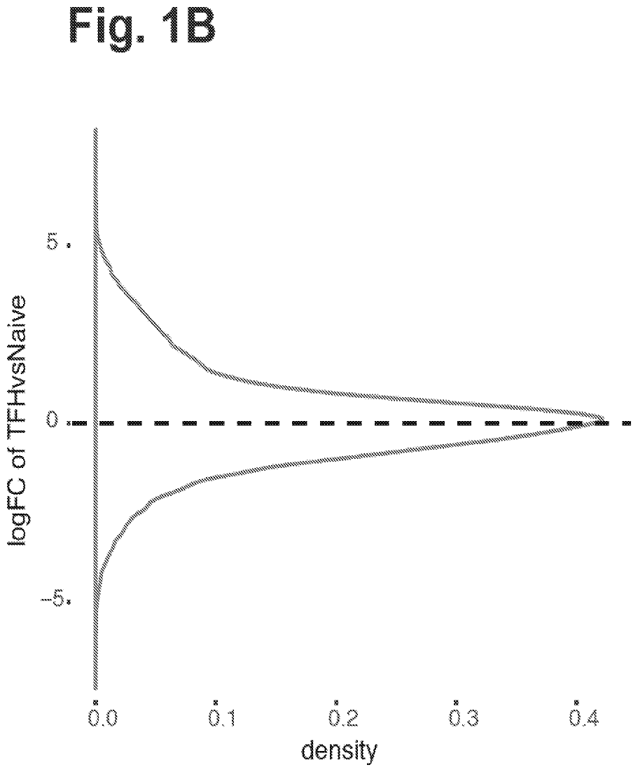

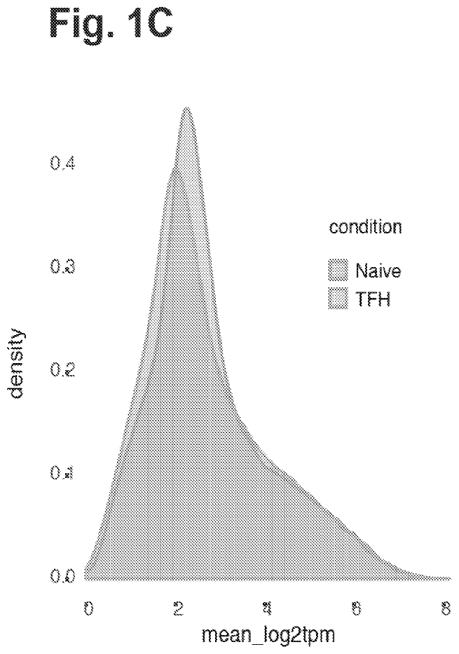

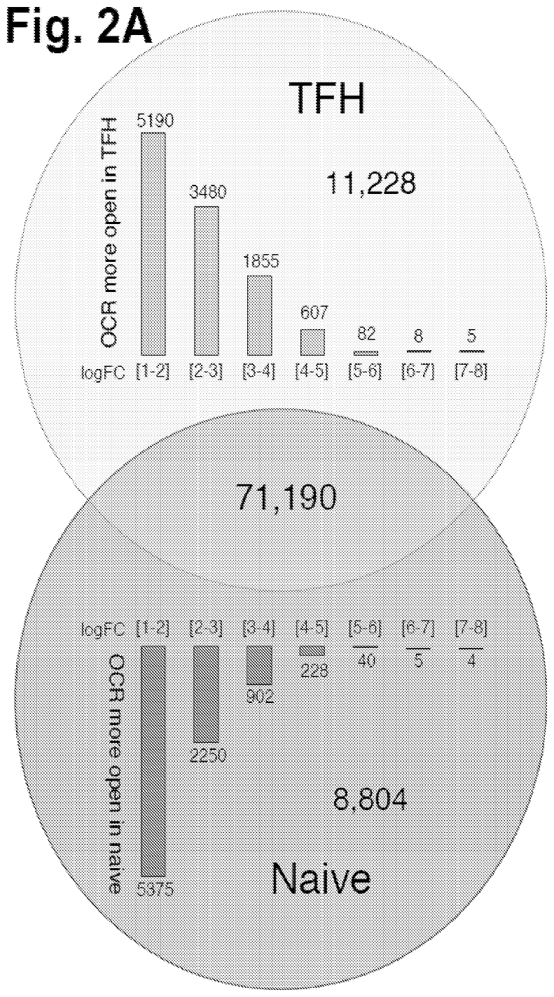

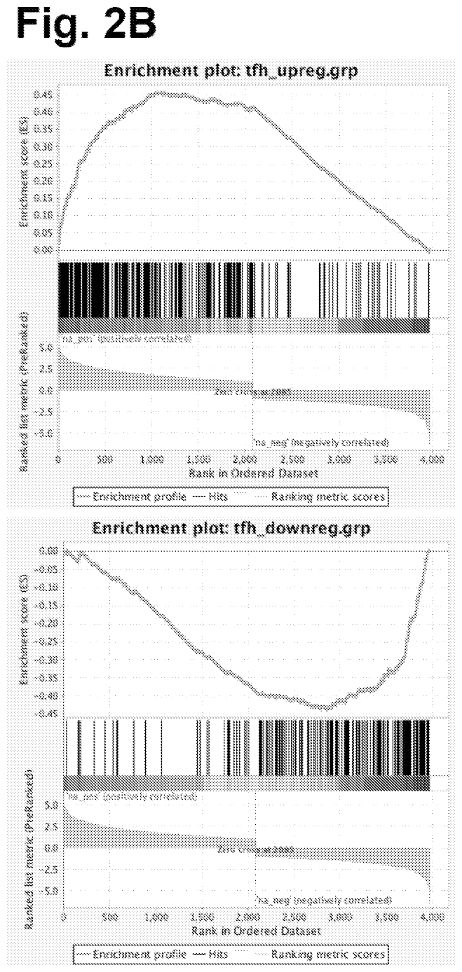

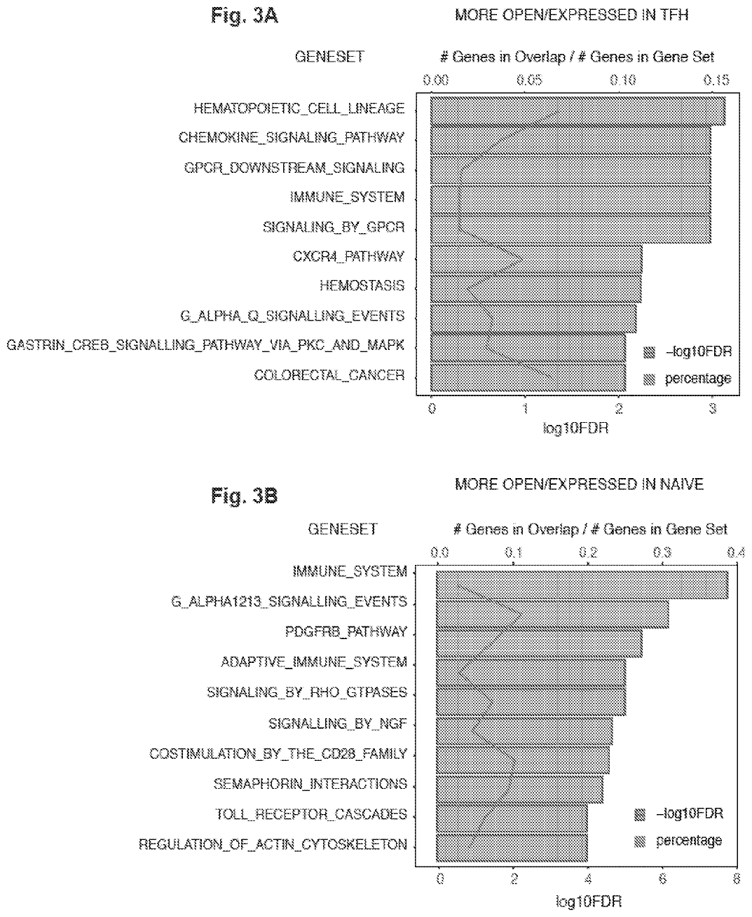

The vast majority (>90%) of the human genome is packed tightly into cellular chromatin and is not accessible to the nuclear machinery that regulates gene expression⁹. Consequently, >95% of transcription factor and RNA polymerase occupancy is concentrated at regions of open chromatin⁹, and thus a map of accessible chromatin in a given cell type essentially defines its potential gene regulatory landscape. As a step toward defining the disease-associated regulatory architecture of the complex heritable autoimmune disease SLE, we focused on human follicular helper CD4+ T cells (TFH), which are required for the production of pathogenic antibodies by autoreactive B cells during the development of SLE⁴. Tonsillar TFH are derived from naïve CD4+ T cell precursors, and represent a population of cells in healthy subjects that are actively in the process of helping B cells to produce high-affinity, class-switched antibodies. We sorted naive CD4+CD45RO− T cells and differentiated CD4+CD45RO+CD25−CXCR5^{hi}PD1^{hi}TFH¹⁰ from human tonsil and generated open chromatin maps of both cell types from three donors using ATAC-seq¹¹. A binary peak calling approach identified a total of 91,222 open chromatin regions (OCR), 75,268 OCR in naïve CD4+ cells and 74,627 OCR in TFH cells (not shown). Further quantitative analysis of the accessibility signal at these OCR revealed a similar overall degree of genomic accessibility (˜1.4%) in both cell types (FIG. 1). However, the differentiation of naïve CD4+ T cells into TFH is associated with remodeling of 22% of the T cell open chromatin landscape, with 11,228 OCR becoming more accessible, and 8,804 OCR becoming less accessible, in the TFH lineage (FIG. 2A). Not only do TFH exhibit more differentially accessible regions than naïve cells, but TFH are also enriched for regions with higher fold-change in accessibility compared to those in naïve cells (FIG. 2A). Among all 20,032 differentially accessible regions, 20.5% (4100) reside in promoters, and the genes driven by these differentially accessible promoters tended to be differentially expressed between TFH and naïve CD4+ cells as assessed by microarray (FIG. 2B, GSEA enrichment p<0.05, absNES>3.5, not shown). The functions of genes that are remodeled and upregulated in TFH were significantly enriched for CD28 costimulatory, G-protein, Rho GTPase, semaphorin, and TLR signaling pathways (hypergeometric test, FDR<0.05, FIG. 3A). Conversely, gene promoters that become less accessible upon TFH differentiation are enriched for pathways involved in chemokine and G protein-coupled signaling (FIG. 3B). These data show that global chromatin remodeling dynamics faithfully reflect dynamic changes in gene expression during the differentiation of follicular helper T cells from their naïve precursors.

### Open Chromatin Mapping of Disease-Relevant Tissue Implicates Causal Disease Variants

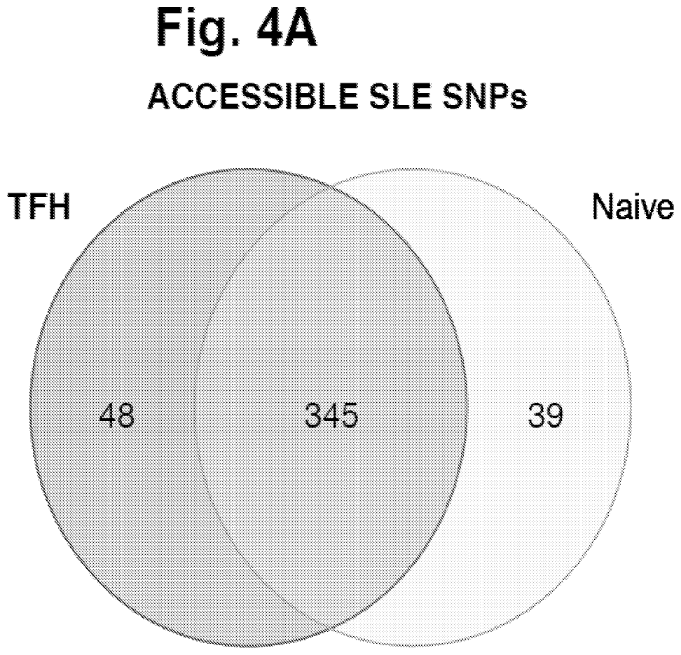

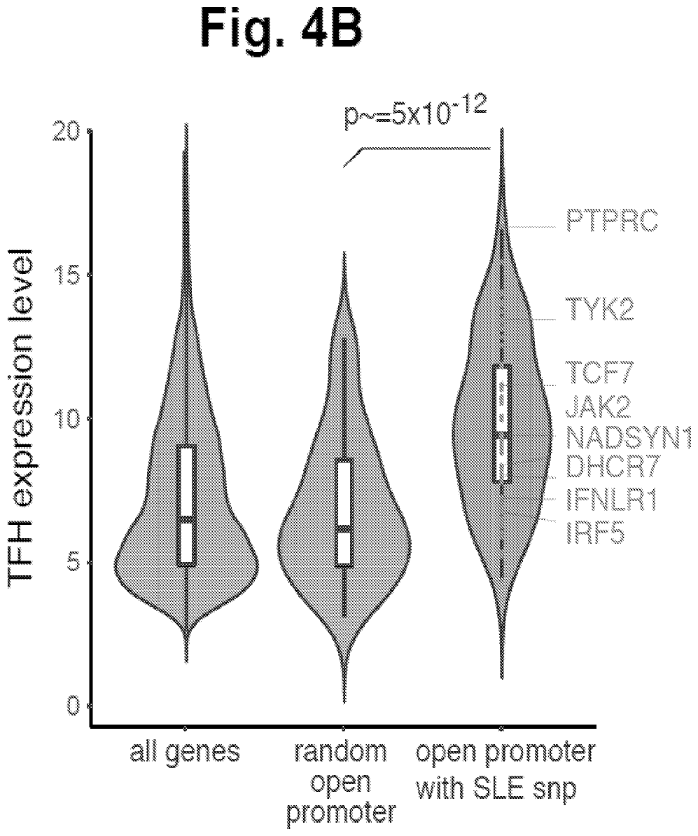

Of the 7662 sentinel and proxy SNPs currently implicated by GWAS in SLE (r²>0.4)^{7,8}, 432 (5.6%) reside in 246 open chromatin regions in either naïve CD4+ T cells or TFH (not shown). Of these, 345 SNPs (80%) are in open chromatin shared by both cell types, 39 are in naive-specific OCR, and 48 reside uniquely within the TFH open chromatin landscape (FIG. 4A). Altogether, 91% (393 of 432) of the accessible SLE SNPs identified in this study reside in the open chromatin landscape of TFH cells. To explore the potential significance of these open chromatin-implicated variants, we first focused on the 132 SLE proxy SNPs that reside in open promoters of protein-coding genes in TFH. Of the 64 genes containing one or more open promoter variants, 62 are expressed in TFH (not shown). Moreover, the set of genes with accessible SLE variants in their promoters are expressed more highly in TFH than the set of all genes, or compared to a random sample of genes with open promoters in TFH (FIG. 4B). Eighty-three SLE proxy SNPs reside in promoters of 36 genes in the top 75% expression quantile, and 43 SLE proxy SNPs reside in the promoters of 18 genes in the 50-75% expression quantile. Thus, nearly 93% (123 of 132) of open promoter SLE proxy variants are positioned at genes moderately to highly expressed in TFH. Ingenuity pathway analysis (IPA) found that these TFH expressed genes are enriched for factors involved in systemic lupus erythematosus (P=4.6×10⁻⁹), systemic autoimmunity (p=6.0×10⁻⁸), and rheumatic disease (P=2.7×10⁻⁷) (FIG. 4C), including PTPRC (CD45), TCF7 (TCF1), IRF5, IFNLR1, TYK2, ELF1, IKZF2 and JAK2. This set of highly-expressed TFH genes with SLE variants in their promoters also includes DHCR7 and NADSYN1, enzymes involved in biogenesis of vitamin D, a process known to play an important role in autoimmune disease susceptibility¹². These results indicate that open chromatin landscapes in disease-relevant cell types represent a highly specific filter through which putatively functional SLE variants can be distinguished from the thousands of proxy SNPs to the sentinels implicated by GWAS.

### Analysis of the Three-Dimensional Promoter Connectome Structure in TFH Cells

It is relatively clear how genetic variation at a promoter might influence expression of the downstream gene, however, ˜70% of the accessible SLE SNPs in TFH cells are intronic and intergenic. How these variants regulate the expression of specific TFH genes is not clear from 1-dimensional open chromatin mapping alone. To explore the role that accessible, non-coding SLE-associated variants play in the disease-related regulatory architecture of the human genome, we derived genome-wide, three-dimensional promoter contact maps of naive CD4+ T cells and differentiated follicular helper CD4+ T cells from human tonsil using promoter-focused Capture-C technology. Our chromosome conformation capture (3C) approach is a large-scale, high-resolution modification of Capture-C¹³ that involves massively parallel, hybridization-based enrichment of 41,970 targeted promoters associated with 123,526 currently annotated transcripts (gencode v19) covering 89% of protein-coding mRNA genes and 59% of non-coding (anti-sense RNA, snRNA, miRNA, snoRNA and lincRNA) genes in the human genome¹⁴. As in standard capture-C approaches, valid hybrid reads derived from ligation of distant fragments with bait fragments were preprocessed using HiCUP¹⁵, and significant promoter-interacting regions (PIR, score >5) were called using CHiCAGO¹⁶. Unlike promoter capture Hi-C¹⁷, our method employs the 4-cutter DpnII to generate 3C libraries with a 270 bp median resolution, ˜9-fold higher than the 2300 bp median resolution of the HindIII-based 3C libraries generated in HiC and capture-HiC approaches. This resolution allows mapping of interactions between promoters and distal regulatory elements to within a span of two nucleosomes. This precision comes at the expense of power, in that sequencing reads are distributed across more fragments, leaving fewer reads available per fragment to call significant promoter interactions. To circumvent this problem, we called promoter interactions both at high resolution (single-fragment) and at lower resolution (four-fragment) after an in silico fragment concatenation step. Combination of both sets of calls allows this method to benefit from the precision of single DpnII fragment analysis and the power of lower resolution analyses at farther distances to assemble comprehensive, 3D promoter contact maps for the human genome (FIG. 5A).

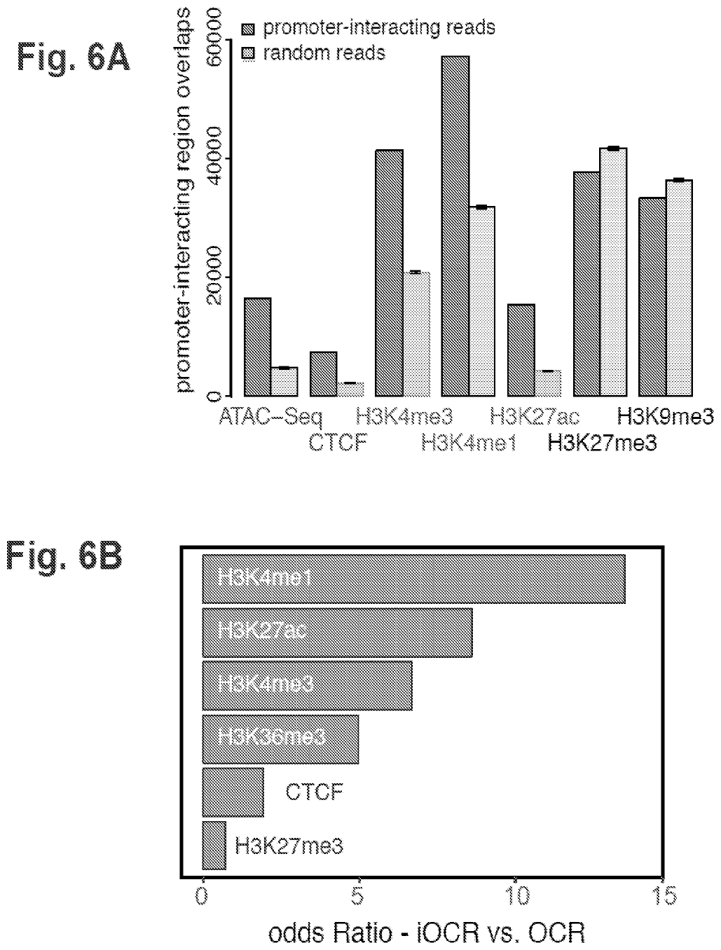

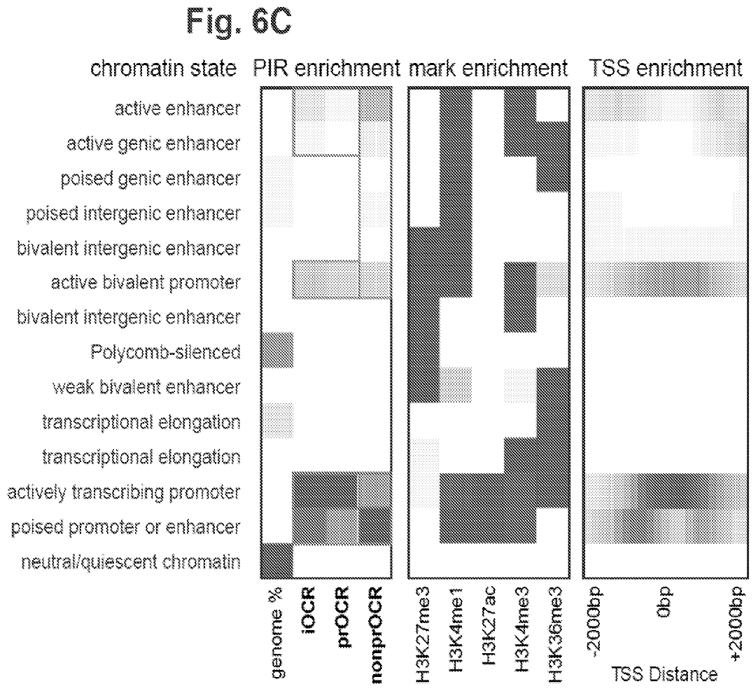

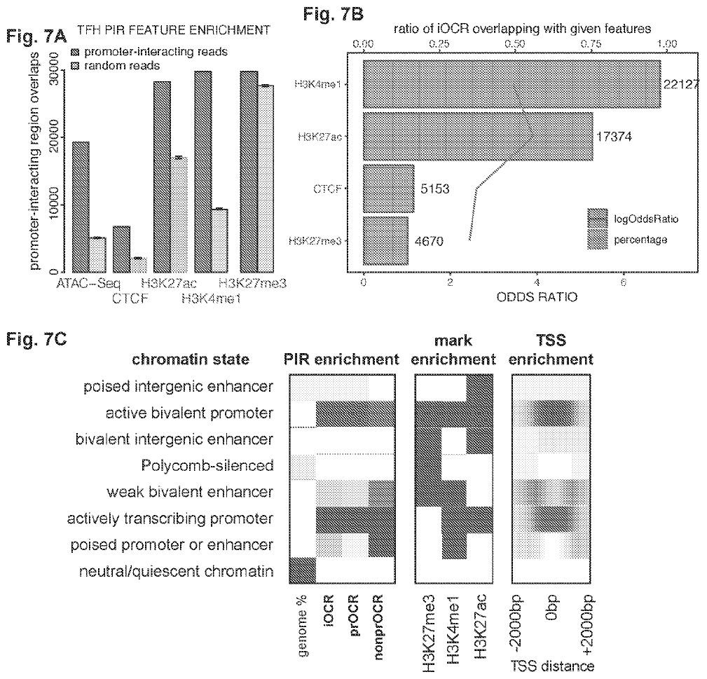

We detected a similar number of significant promoter interactions (CHiCAGO score >5) in both cell types—255,238 in naive CD4+ T cells and 224,263 in TFH—with the vast majority (>99%) being intra-chromosomal (in cis). About 20% of total interactions were between two promoters, while 80% of interactions were between a promoter and an intergenic or intronic region (not shown). We observed significant variability in the 3D structure of individual promoters across all genes in naïve cells and TFH. We were unable to detect consistent interactions at 30% of captured promoters in both cell types, while 70% of promoters were found engaged in at least one stable interaction with another genomic region. Of these promoters, over 80% were connected to only one distal genomic region, indicating that most promoters in these cell types exhibit very low spatial complexity. However, ˜1% of all promoters exhibited significant spatial complexity, interacting with four or more distal regions, with some promoters engaging in as many as 70 interactions with distal regions. The number of connections per promoter correlated with the level of gene expression in both cell types, with the most interactive promoters belonging to highly-expressed genes with known roles in TFH function (FIG. 5B). Two examples are the IL21 and IFNG promoters, which are expressed and show complex connectomes in TFH but not naïve cells (FIG. 5C and FIG. 5D). Promoter-interacting regions in both cell types were enriched 3-fold for open chromatin, and 2-fold for chromatin signatures associated with active transcription, such as H3K27ac, H3K4me1, H3K4me3 (FIG. 6A and FIG. 7A). Conversely, PIR in both cell types were depleted of the silencing marks H3K27me3 and H3K9me3 (FIG. 6A and FIG. 7A). Together, these trends indicate that the promoter contacts captured by this approach preferentially represent the regulatory architectures of the associated genes.

### The Promoter-Open Chromatin Connectome of TFH Cells

To further explore the regulatory nature of the spatial connections between promoters and other genomic regions in the nucleus, we focused on interactions between promoters and open chromatin regions, as the biochemical processes that regulate transcription occur largely at accessible DNA⁹. Instead of using standard fragment-based interactions¹³, we used a feature-based calling approach to define interactions between promoters (−1500 to +500 from a TSS) and OCR, combining calls at both one- and four-fragment resolution to generate genome-wide, open chromatin-promoter interaction landscapes in naïve and follicular helper CD4+ T cells (FIG. 5A). In total, we detected 71,137 cis-interactions between accessible promoters and open chromatin among both cell types, involving 34% of the total open chromatin landscape (31,404 OCR) identified by our ATAC-seq analyses (not shown). Roughly half of promoter-interacting OCR (15,109, 48%) are located in intergenic or intronic regions relatively far from genes, while the other 16,295 promoter-interacting OCR (52%) are located in the promoters of other distant genes, representing promoter-promoter interactions. The distance between promoter and promoter-interacting OCR pairs ranged from a few hundred base pairs to over a megabase, with a median of ˜112 kb for both categories. Remarkably, while OCR in general are enriched for active chromatin marks^{9,11}, we find that promoter-connected OCR are even more highly enriched for enhancer signatures compared to OCR that are not engaged in promoter interactions (14-fold for H3K4me1, 9-fold for H3K27ac, 7-fold for H3K4me3, Fisher test p<2×10⁻¹⁶, FIG. 6B and FIG. 7B. Chromatin state modeling (chromoHMM¹⁸) revealed that all promoter-interacting OCR (iOCR) were enriched at active promoters, bivalent promoters, and active enhancers, as defined by histone modification ChIP-seq in both naïve and TFH cells¹⁹⁻²¹ (FIG. 6C and FIG. 7C). Open chromatin regions involved in promoter-promoter interactions (prOCR) were more specifically enriched with active promoter signatures, while promoter-interacting OCR located in intergenic/intronic space (nonprOCR) were more specifically enriched at poised and active enhancers (FIG. 6C). These results indicate that promoter-connected OCR are biochemically distinct from OCR not connected to a promoter, and that this promoter-Capture-C approach enriches for genomic elements that are actively engaged in gene regulation.

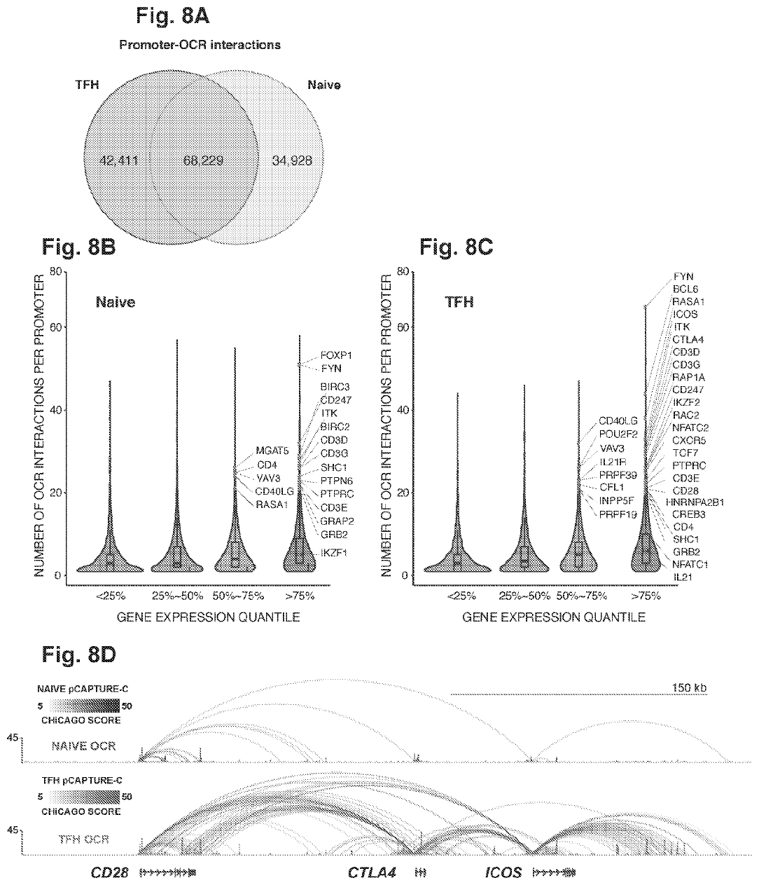

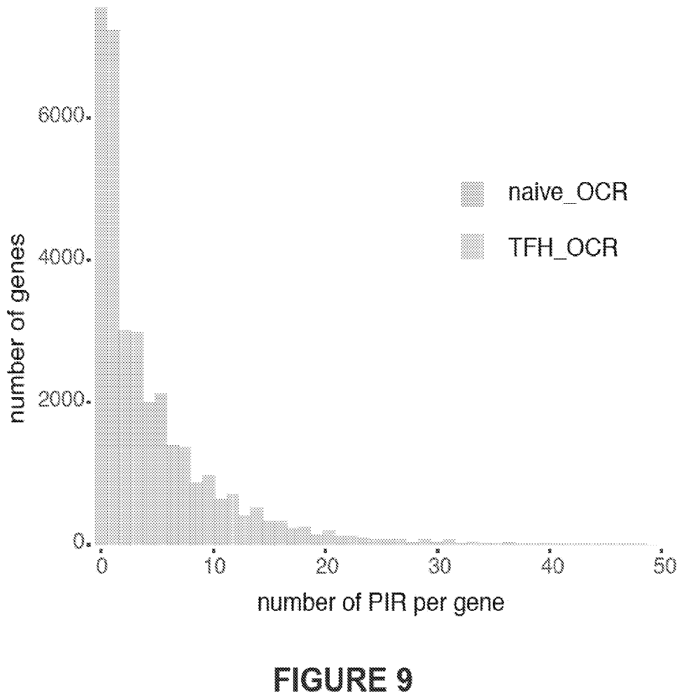

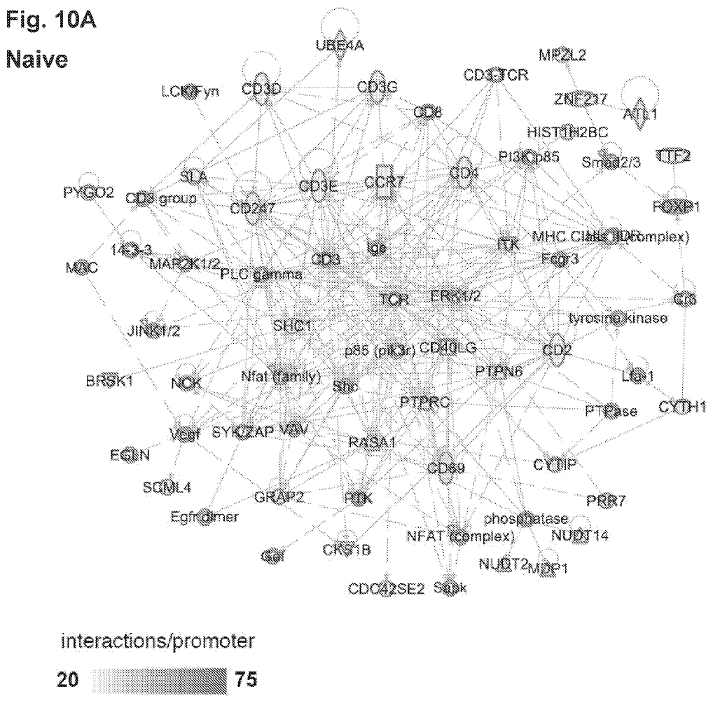

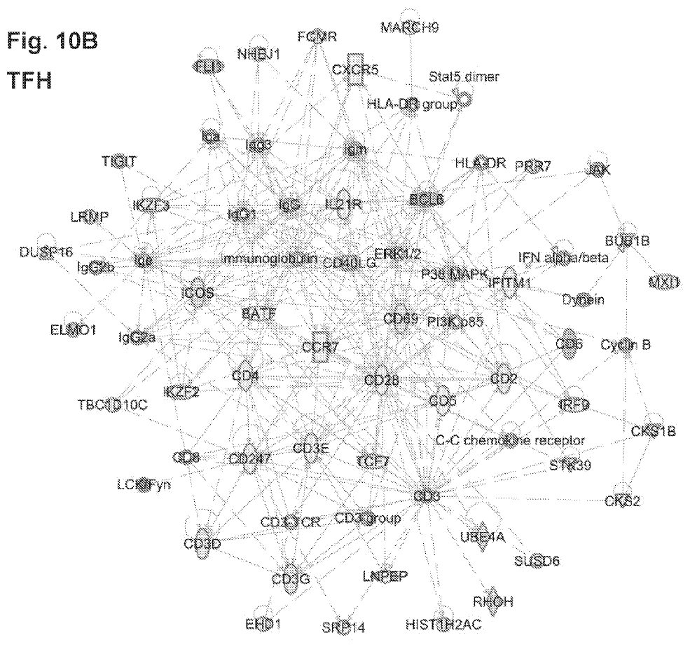

Using this open chromatin-promoter interaction landscape, we were able to connect the promoters of 18,669 genes (associated with 79,330 transcripts) to their corresponding putative regulatory elements, representing 145,568 distinct gene-OCR interactions. Roughly half (47%, 68,229) of these interactions occur in both naive and TFH cells, while 24% (34,928) occur uniquely in naïve cells, and 29% (42,411) are only found in TFH (FIG. 8A). Overall, 91% of OCR-connected genes (17,021) exhibit at least one differential promoter-OCR interaction in naïve vs. follicular helper T cells. The majority (82%) of OCR-connected genes were incorporated into regulatory structures consisting of more than one distal regulatory region in naïve and follicular helper T cells. On average, each of these connected genes interact with 6 OCR in both naïve CD4+ T cells and follicular helper T cells (4 in median, FIG. 9), with 10% of these genes involved in 13 or more interactions with distal OCR. More interestingly, the degree of spatial connectivity exhibited by a promoter tends to positively correlate with the level of gene expression in a lineage-specific manner (FIG. 8B and FIG. 8C). The common, highly-connected promoters in both cell types drive the expression of genes involved in cell cycle, DNA organization and repair, protein and RNA biogenesis and trafficking, and TCR signaling (FIG. 10). In addition, highly interactive promoters in naïve cells are involved in quiescence, signal transduction and immune function (e.g., FOXP1, CCR7, IKZF1, CD3, FYN, GRB2, GRAP2, BIRC2/3; FIG. 8B), while gene promoters that exhibit complex regulatory architectures in TFH are highly expressed in TFH and are involved in TFH and T cell differentiation, survival, homing, and function (e.g., BCL6, CXCR5, CD40L, CTLA4, ICOS, CD2, CD3, CD28, CD69, TCF7, NFAT1, BATF, ITK, IKZF2, IKZF3, IL21R, FAS; FIG. 8C). An example is the CD28-CTLA4-ICOS multi-locus region. In naïve CD4+ T cells, which express CD28 but not CTLA4 or ICOS, the CD28 promoter is engaged in multiple interactions with 8 downstream regions of open chromatin (FIG. 8D, blue), while the CTLA4 and ICOS promoters are much less interactive. In TFH, which express all three genes, the CD28, CTLA4, and ICOS promoters adopt extensive, de novo spatial conformations involving contacts with more than two dozen putative regulatory elements within the TFH-specific open chromatin landscape (FIG. 8D, red). Together, these results reveal major restructuring of the T cell gene regulatory architecture that occurs as naïve helper T cells differentiate into follicular helper T cells, and indicate that complex, three-dimensional regulatory architectures are a feature of highly expressed, lineage-specific genes involved in specialized immune functions in this disease-relevant cell types.

### Disease-Associated Variant-to-Gene Mapping for SLE

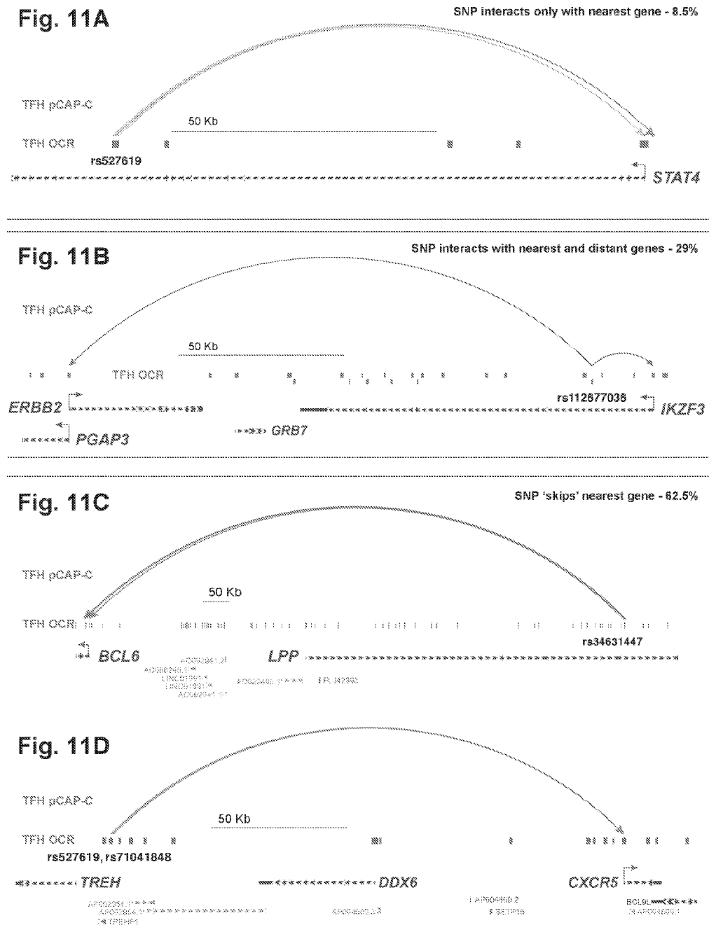

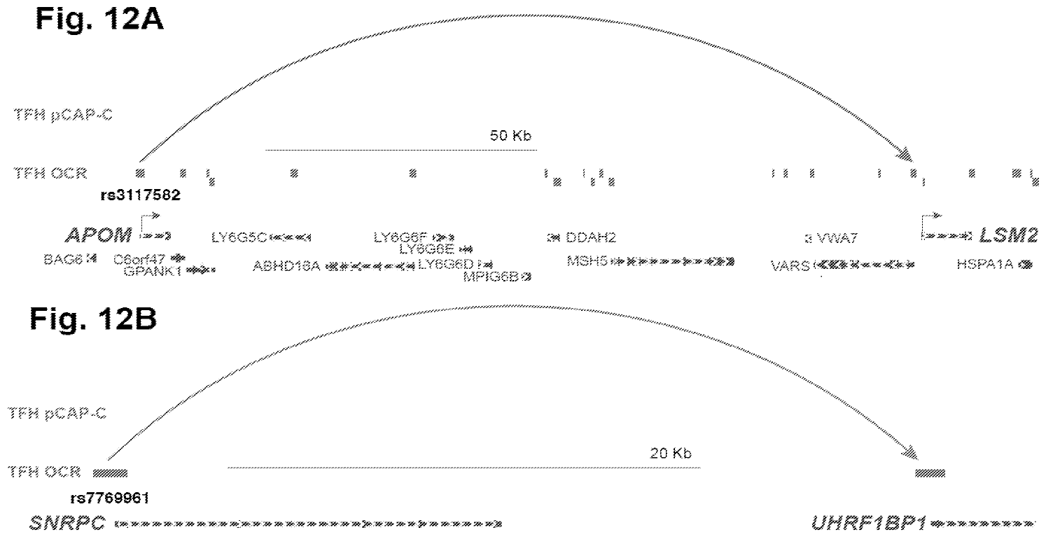

The open chromatin landscape of follicular helper T cells contains 393 accessible genomic regions that harbor SLE disease variants (not shown), representing the TFH component of the potential cis-regulatory landscape of SLE. While 33% of accessible variants (132 proxy SNPs) reside in promoters, 67% of accessible SLE SNPs (N=261) are in non-promoter regions. Therefore, the role these regions might play in gene transcription, and which genes they might control, is not clear from these one-dimensional epigenomic data. To determine whether spatial proximity of a gene to an open SLE SNP in three dimensions is a predictor of its role in TFH and/or SLE, we explored the 3D cis-regulatory architecture of SLE genetic susceptibility based upon open chromatin region interaction landscape generated in TFH cells, effectively mapping 256 open SLE variants (69 sentinels, r²>0.4) to 330 potential target genes (1107 SNP-target gene pairs). This 3D variant-to-gene map shows that only ˜9% (22) of the SLE variants that reside in TFH open chromatin interact exclusively with the nearest gene promoter (Table 1). An example of this category is rs35593987, a proxy to the SLE sentinel SNP rs11889341 and rs4274624 that resides in a TFH OCR and loops ˜99 kb to interact with the STAT4 promoter (FIG. 11A). Another ˜30% (75) of open SLE variants interact with nearest promoter, but also with the promoters of more distant genes (FIG. 11A, Table 1). An example of this category is rs112677036, a proxy to the SLE sentinel SNP rs12938617 that resides in the first intron of IKZF3, interacts with nearby IKZF3 promoter, but also interacts with promoters of two 157 kb upstream genes PGAP3 and ERBB2 (FIG. 11B). Remarkably, over 60% of all open SLE variants (159) ‘skip’ the nearest gene to interact with at least one distant promoter (Table 1). Examples of this most abundant category are rs34631447, a proxy to the SLE sentinel rs6762714 SNP that resides in open chromatin in the sixth intron of the LPP locus, and the intergenic rs527619 and rs71041848 SNPs proxy to SLE sentinel rs4639966. Our 3D regulatory map in TFH cells demonstrates that the ‘LPP’ variant in fact does not interact with the LPP promoter, but instead is incorporated into a chromosomal loop structure spanning over 1 Mb that positions it in direct, spatial proximity to the promoter of BCL6, the ‘master’ transcription factor of follicular helper T cells²²⁻²⁶ (FIG. 11C). Similarly, the OCR containing the SLE proxies rs527619 and rs71041848 does not interact with the nearby TREH gene, but instead loops to interact with the promoter of the TFH-specific chemokine receptor gene CXCR5, nearly 200 kb away (FIG. 11D). Other relevant examples of this class of SLE SNPs are rs3117582 and rs7769961, proxies to SLE sentinel SNP rs1150757 and rs9462027, respectively. These SNPs in TFH open chromatin loop 35 to 150 kb to interact with LSM2 and SNRPC (FIG. 12), both of which encode proteins that participate in the processing of nuclear precursor messenger RNA splicing, and are frequently the targets of autoantibodies produced by patients with SLE^{27,28}.

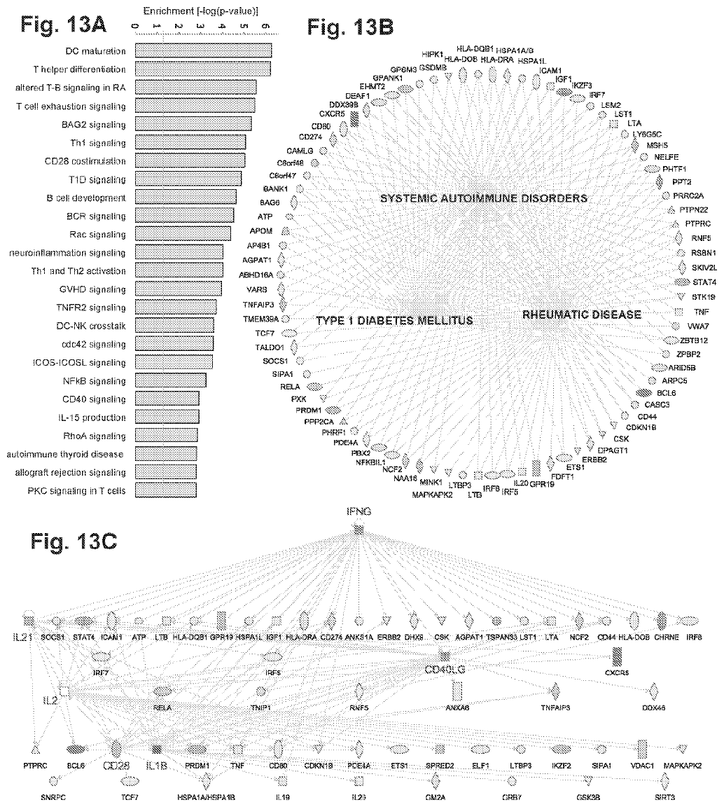

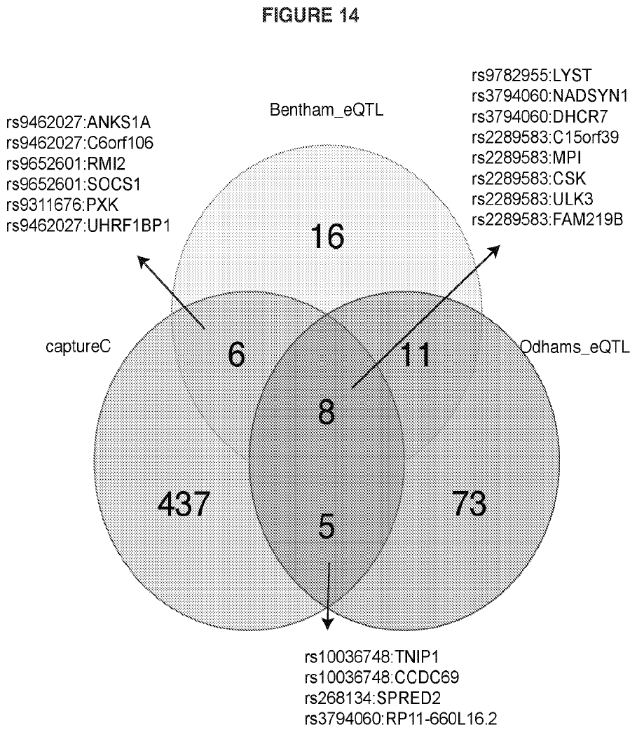

Ontology of the set of genes found physically connected to open SLE variants showed enrichment for pathways involved in dendritic cell maturation, T-B cell interactions, T helper differentiation, NFkB signaling, and costimulation through CD28, ICOS, and CD40 (FIG. 13A). The top three disease networks enriched in SLE SNP-connected genes are systemic autoimmune disorders, rheumatic disease, and type 1 diabetes, all inflammatory disorders involving autoantibody-mediated pathology (FIG. 13B). At least 200 of these connected genes are differentially expressed between naïve and follicular helper T cells (Table 1), and many have known roles in TFH and/or T cell function (e.g., BCL6, CXCR5, TCF7, PRDM1, IKZF3, IKZF2, IRF8, ETS1, ELF1, EBI3, PTPN22, PDL1, TET3, IL19, IL20). Similarly, SLE SNP-connected genes are highly regulated (P<10⁻⁶) in a hierarchical manner by IFNγ, IL-2, IL-21, IL-1, IL-27, CD40L, and TCR/CD28 (FIG. 13C). We also compared our list of genes found physically associated with SLE SNPs in TFH with those found statistically associated with SLE variants through eQTL studies in two distinct human subject cohorts. One study by Odhams et al. identified 97 gene-SNP eQTL in B-LCL lines²⁹, while another by Bentham et al. used B-LCL lines and undifferentiated leukocyte subsets from peripheral blood to identify 41 SLE eQTL⁷. Over one-third (14/41) of the SNP-gene associations implicated by the Bentham study were also identified by our promoter-Capture-C approach in tonsillar TFH cells from three healthy donors (ANKS1A, C6orf106, RMI2, SOCS1, PXK, UHRF1BP1, LYST, NADSYN1, DHCR7, C15orf39, MPI, CSK, ULK3, FAM219B; FIG. 14). Similarly, 13% (13/97) of the genes implicated by Odhams et al. were found connected to the same SLE SNPs in tonsillar TFH cells (LYST, NADSYN1, DHCR7, C15orf39, MPI, CSK, ULK3, FAM219B, TNIP1, CCDC69, SPRED2, RP11, FIG. 14), for a total of 16% (19/119) SNP-gene pairs overlapping between both studies. These results indicate that a gene's spatial proximity to an accessible, disease-associated SNP in 3D is a strong predictor of its role in the context of TFH biology and SLE disease pathogenesis.

### Genomic Regions Identified by SLE GWAS and 3D Epigenomics Regulate Major TFH Genes

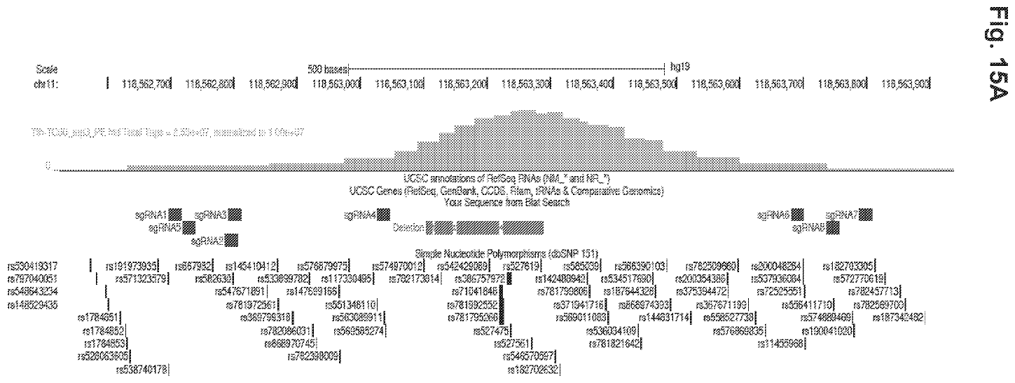

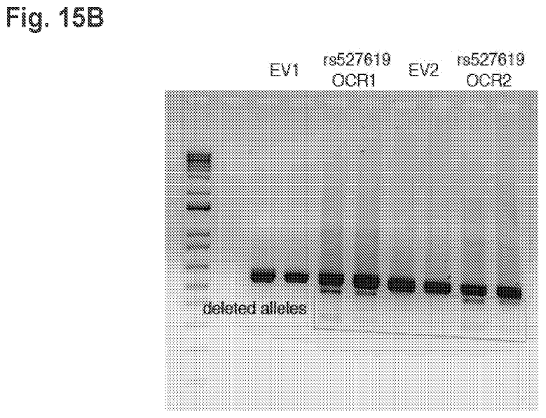

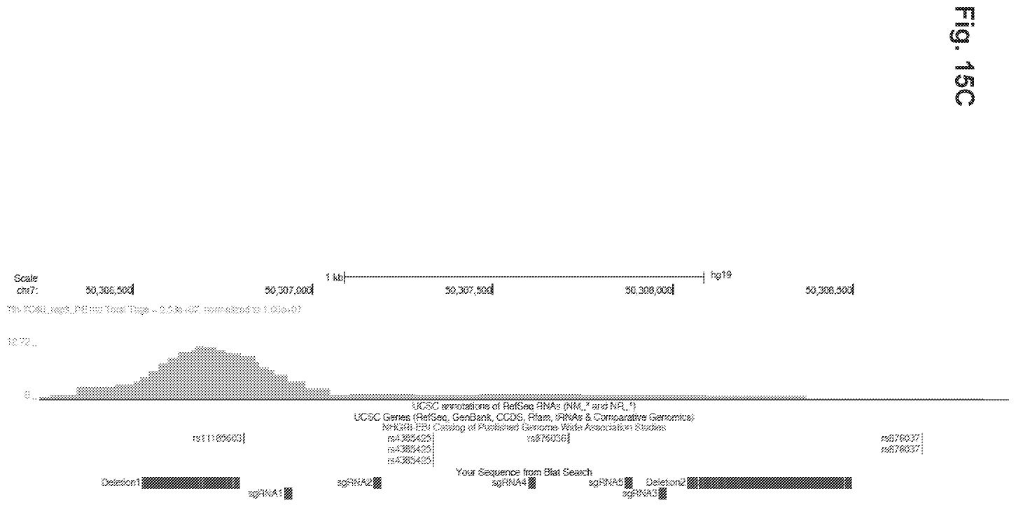

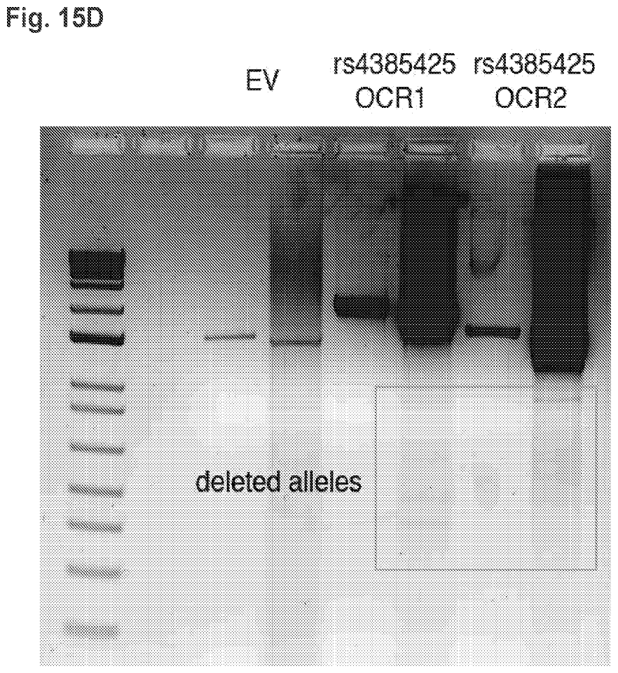

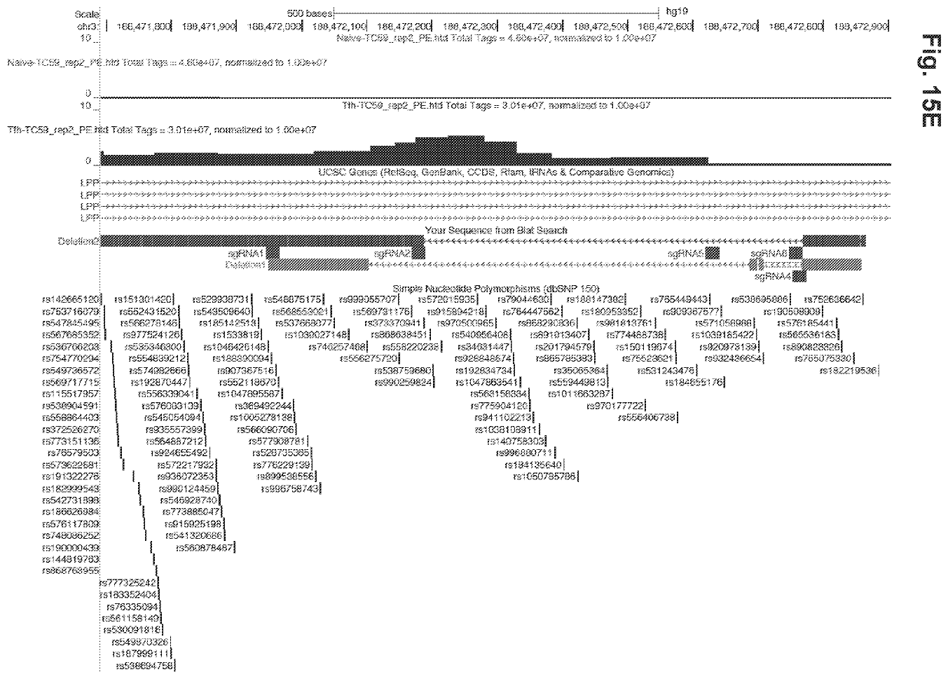

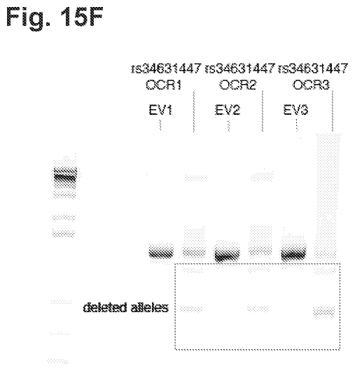

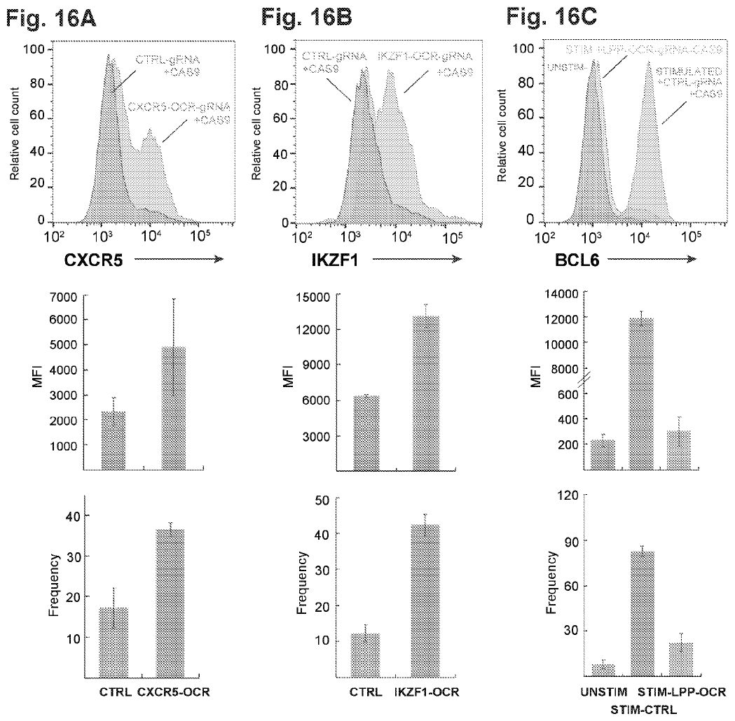

To validate that genomic regions implicated by ATAC-seq, promoter-Capture-C, and SLE-associated genetic variation function as bona fide distal regulatory elements for their connected promoters, we used CRISPR/CAS9 to specifically delete several OCR harboring SLE variants from the Jurkat T cell genome. We first targeted the intergenic region near the TREH gene that harbors the rs527619 and rs71041848 proxies to the rs4639966 SLE sentinel SNP, and was captured interacting with the CXCR5 promoter (FIG. 15). Neither untargeted parental Jurkat cells nor a control-targeted Jurkat line express CXCR5, but deletion of this region led to induction of CXCR5 in approximately half of the cells (FIG. 16A). Similarly, parental and control-targeted Jurkat cells do not express IKZF1, which encodes the transcription factor Ikaros, but deletion of the OCR containing the rs4385425 proxy SNP to the sentinel SLE SNP rs11185603 (FIG. 15) induced expression of Ikaros in nearly half of the cells (FIG. 16B). We also targeted the TFH-specific open chromatin region in the sixth intron of the LPP gene (FIG. 15) that harbors the rs34631447 and rs79044630 SNPs proxy to sentinel rs6762714 SLE SNP, and was observed interacting with the promoter of BCL6. BCL6 is not expressed by parental or control-targeted Jurkat cells, but is induced by IFN-gamma (FIG. 16C). However, inducible expression of BCL6 was completely abrogated in Jurkat cells lacking the ˜150 bp SLE-associated LPP OCR (FIG. 16C). These results confirm that these distal SLE-associated regions, which are located hundreds to thousands of kilobases away in one dimension, interact with and act as crucial regulatory elements for the genes encoding the master TFH transcription factor BCL6, the IKZF1 transcriptional repressor, and the TFH chemokine receptor CXCR5. These results indicate that the 3D promoter connectomes detected in these cells reveal bona fide gene regulatory architectures.

### SLE-Associated Open Chromatin Promoter Connectomes Implicate Novel Genes Involved in TFH Function

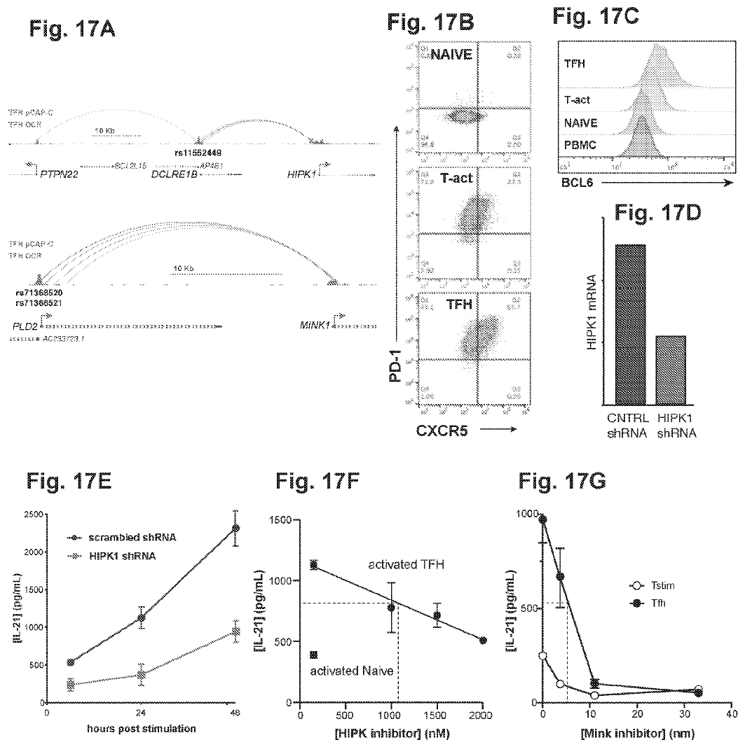

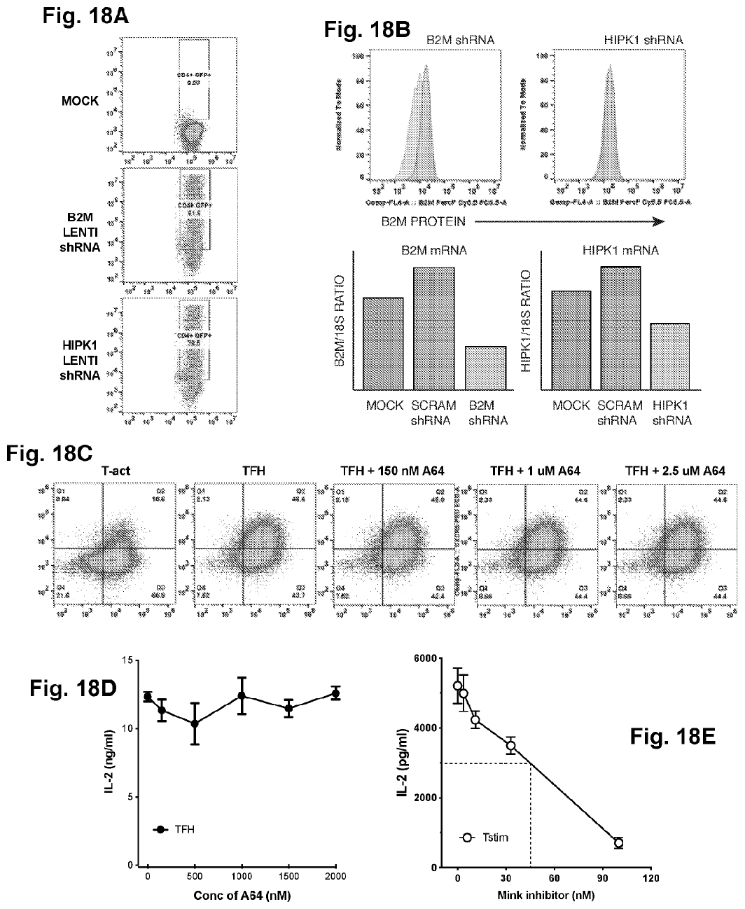

From the set of 243 promoter-connected open SLE variants in TFH cells, we noted a subset of variants that skipped nearby promoters to interact with genes that are upregulated upon TFH differentiation, but have no known specific role in TFH biology. These genes are enriched in canonical pathways such as mannose degradation (MPI), epoxysqualene biosynthesis (FDFT1), di- and tri-acylglycerol biosynthesis (LCLAT1, AGPAT1), cholesterol biosynthesis (DHCR7, FDFT1), oxidized GTP/dGTP detoxification (DDX6), breast and lung carcinoma signaling (ERRBB2, HRAS, RASSF5, CDKN1B), tRNA splicing (TSEN15, PDE4A), pentose phosphate pathway (TALDO1), acetyl-coA biosynthesis (PDHB), dolichyl-diphosphooligosaccharide biosynthesis (DPAGT1), and valine degradation (HIBADH). Two of these genes, HIPK1 and MINK1 (FIG. 17A), encode a homeobox-interacting kinase and a MAP3/4K homolog that each regulate gene expression in other cell types^{30,31}. Like many genes in this category, both HIPK1 and MINK1 are upregulated in TFH, and their promoters interact with OCR that are genetically associated with SLE risk, suggesting they are involved in TFH function. To test this, we transduced TFH differentiated in vitro from naïve CD4+ T cells³² (FIG. 17B and FIG. 17C) with a lentiviral vector expressing shRNA targeting the HIPK1 transcript to knock down HIPK1 expression (FIG. 17D), or with scrambled or B2M shRNA as controls (FIG. 18). GFP+ cells were sorted, re-stimulated with CD3/28 beads, and secretion of IL-21, the major cytokine required for T cell help for B cell antibody production, was measured in the supernatant by ELISA. Remarkably, targeting of HIPK1 expression had no effect on in vitro TFH differentiation as measured by induction of BCL6, PD-1 or CXCR5 (FIG. 18), but resulted in a ˜3-fold decrease in IL-21 production (FIG. 17E). To determine if pharmacologic targeting of HIPK1 can also modulate TFH function, we treated in vitro differentiated TFH with the HIPK1/2 inhibitor A64. As with genetic targeting, pharmacologic inhibition of HIPK activity resulted in a dose-dependent reduction in IL-21 production by activated TFH cells (FIG. 17F) without effecting proliferation, viability, or differentiation (FIG. 18). We also observed that inhibition of HIPK1 inhibits expression of PD1, IL6 receptor, IL2 receptor, BACH2, SPRED2, ARID5B and PTPN22, all genes associated with the SLE phenotype. As a further test of whether SLE-associated promoter-OCR connectomes can reveal novel drug targets for TFH function, we targeted MINK1 pharmacologically with the MAP3/4K antagonist PF06260933. Treatment with this inhibitor resulted in a dose-dependent reduction in IL-21 secretion by TFH cells, with an ED₅₀ of 5 nM (FIG. 17G). Unlike the HIPK1 inhibitor, this MINK1 inhibitor did impact T cell IL-2 production and proliferation, but with an ED₅₀ 8- to 10-fold higher than its effect on IL-21 production (FIG. 18).

We have also identified two families that carry HIPK1 mutations that are associated with clinical immunophenotypes in homozygous children. We are generating EBV-transformed B cell lines from these patients. We will also delete HIPK1 using CRISPER/CAS9 in normal EBV-transformed B cell lines and assess the impact on immune associated gene expression programs using RNA-seq. These lines can also be used to advantage for re-expression wild type or mutant HIPK1 to assess the same in functional studies.

As demonstrated above, these data show that integrated, 3-dimensional maps of disease-associated genetic variation, open chromatin, and promoter connectomes can lead to bona fide novel drug targets that control tissue-specific and disease-relevant biology.

### Discussion

In this study, we used systems-level integration of disease-associated genetic variation and 3-dimensional epigenomic maps of the interactions between open chromatin and promoters in a highly disease-relevant tissue to identify putative disease-associated regulatory regions and the genes they influence. Our one-dimensional open chromatin analyses demonstrated a strong correlation between promoter accessibility and differential gene expression in human tonsillar naive vs. follicular helper T cells. These analyses also showed that SLE-associated variants in accessible promoters in TFH tend to tag highly expressed genes enriched in autoimmune disease pathways, suggesting that TFH open chromatin landscapes represent a useful filter through which functional, systemic autoimmune disease-associated variants can be identified out of the thousands of sentinel and proxy SNPs to the sentinel signals implicated by GWAS. However, only 20% of OCR are located in promoter regions, while 80% of the open chromatin regions in human naive and follicular helper T cells map to non-coding/non-promoter regions of the genome, making an interpretation of the potential regulatory role of these regions challenging. To overcome this problem, we generated high-resolution, comprehensive maps of the open chromatin-promoter connectome in naive and TFH cells, allowing physical assignment of non-coding OCR and SNPs to genes, and revealing the potential regulatory architectures of nearly all coding genes and over half of non-coding genes in human immune cell types with crucial roles in humoral immunity and systemic immunopathology. Similar to previous promoter interactome studies³³, we found that promoter-interacting regions are enriched for open chromatin and the chromatin-based signatures of enhancers. However, we also found that open chromatin regions that interact with a promoter are enriched over 10-fold for enhancer marks compared to OCR that are not connected to a promoter, suggesting that promoter-focused Capture-C preferentially identifies non-coding regions with gene regulatory activity. We also observed enrichment of enhancer marks at open promoters engaged in promoter-promoter interactions compared to promoters not connected to another promoter, suggesting that promoters may synergize in three dimensions in an enhancer-like manner to augment expression of their connected genes.

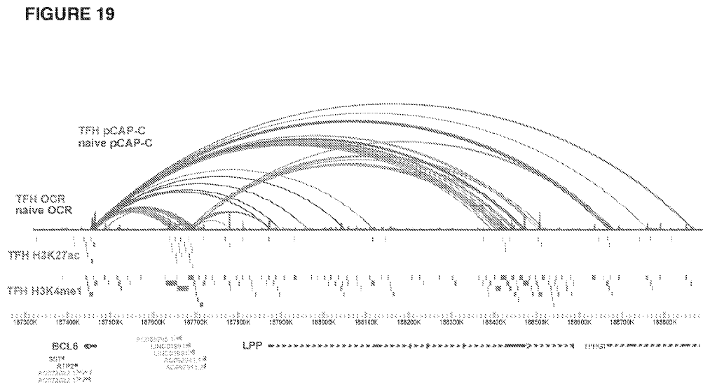

Our study shows that, similar to previous estimates³⁴, less than 10% of promoter interactions exclusively involve the nearest genes. Over 90% of accessible disease variants interact with distant genes, and that over 60% of open variants skip the nearest gene altogether and exclusively interact only with distant genes. Importantly, we were able to validate direct roles for several SLE-associated distal OCR in the regulation of their connected genes (BCL6, CXCR5, IKZF1) using CRISPR/CAS9-mediated editing in human T cells, suggesting that SLE-associated genetic variation at distant loci can operate through effects on genes with known roles in TFH and/or SLE biology. A locus control region ˜130 kb upstream of the BCL6 gene has been defined previously in germinal center B cells³⁵, and we also find evidence for usage of this region by human TFH cells at the level of open chromatin, histone enhancer marks, and long-range connectivity to the BCL6 promoter (FIG. 19). However, we also observe a much more distant ‘stretch’ enhancer within the LPP gene in TFH cells, as evidenced by extensive open chromatin, H3K27 acetylation, and H3K4 mono-methylation (FIG. 19). This region shows extensive connectivity with BCL6 in the 3D architecture of the nucleus, and the 1 Mb distal SLE-associated BCL6 enhancer validated by genome editing in this study is contained within this BCL6 stretch enhancer. This enhancer region is occupied in lymphoid cell lines by NFkB/RelA and POU2F2, both transcription factors known to positively regulate immunoglobulin and inflammatory gene expression {ENCODE project consortium}. Long-range regulatory elements for CXCR5 have not been previously identified, and the −180 kb SLE-associated element in this study is the first validated for CXCR5. Deletion of this element led to increased expression of CXCR5, suggesting that unlike the distal BCL6-LPP enhancer, this element is a silencer in Jurkat cells. Consistent with this finding, this region is occupied by the repressive transcription factors YY1, BHLHE40 and BATF in lymphoid cells {ENCODE project consortium}, but its function in primary TFH cells remains to be determined. These distant SNP-gene regulatory pairs join examples like the FTO and TCF7L2 loci^{1,3}, in which GWAS data were interpreted to implicate the nearest genes, while 3D epigenomics and functional follow-up showed that the disease variants actually reside in elements that regulate the distant IRX3 gene (for FTO) and the ACSL5 gene (for TCF7L2). Our results indicate that a gene's spatial proximity in three dimensions to a regulatory SLE SNP is a strong predictor of its function in the context of TFH biology and SLE disease pathogenesis, and suggest that assumptions that a given genomic feature (e.g., SNP or TF binding motif) or epigenomic feature (e.g., 5meCpG, 5hmCpG, or histone mark) identified by 1D mapping of the human genome regulates the nearest gene are more likely to be incorrect than correct. These data have important implications for the interpretation of all genetic and epigenomic studies in all tissues.

Remarkably, our integrated open chromatin and promoter connectome mapping in tonsillar TFH cells from three healthy individuals identified one-third of the SNP-gene pairs identified by Bentham⁷, and 13% of the SNP-gene pairs identified by Odhams²⁹. These quantitative trait studies require samples from hundreds of individuals, and the data are obtained from blood, B-LCL, or naïve mononuclear leukocytes. However, immune responses do not take place in the blood, and the pathophysiologic aspects of inflammatory disease are mediated by specialized, differentiated immune cell types that are rare or not present in blood. Our approach utilized human follicular helper T cells from tonsil that are ‘caught in the act’ of mediating coordinated in vivo T-B immune responses, and is the same cell type involved in B cell help for autoantibody production in SLE. In addition, this variant-to-gene mapping approach identified ˜10-fold more SLE SNP-gene association than current eQTL studies.

In addition to revealing the previously unknown SLE-associated regulatory architectures of known TFH/SLE genes, we show that the combination of GWAS and 3-dimensional epigenomics can identify genes with previously unappreciated roles in disease biology through their connections with accessible disease SNPs. In a previous study, we implicated the novel gene ING3 by virtue of its interaction with an accessible osteoporosis SNP, and showed that this gene is required for osteoclast differentiation in an in vitro mode¹⁴. In this current study, approximately two dozen ‘novel’ genes up- or down-regulated during differentiation of naive CD4+ T cells into TFH were implicated through their connection to SLE SNPs. Among these are HIPK1, a nuclear homeobox-interacting protein kinase that cooperates with homeobox, p53, and TGFB/Wnt pathway transcription factors to regulate gene transcription^{30,36-38}. A role for HIPK1 in T-independent B cell responses has be identified in the mouse³⁹, but no role for this kinase has been previously established in TFH or SLE. Another gene implicated in our study is MINK1, which encodes the misshapen-like kinase MAP4K6. This kinase functions upstream of JNK and SMAD in neurons^{40,41}, and has been shown to inhibit TGFB-induced Th17 differentiation⁴². However, a role in TFH or SLE has likewise not been previously appreciated. We show that genetic and/or pharmacologic targeting of HIPK1 or MINK1 in human TFH cells inhibits their production of IL-21, a cytokine required for T cell-mediated help for B cell antibody production⁴³. The present example shows the utility of this integrated approach in identifying novel targets for drug repurposing or new compound development in complex heritable diseases.

### REFERENCES

1. Smemo, S. et al. Obesity-associated variants within FTO form long-range functional connections with IRX3. Nature 507, 371 375 (2014).
2. Claussnitzer, M. et al, FTO Obesity Variant Circuitry and Adipocyte Browning in Humans. New Engl J Medicine 373, 895-907 (2015).
3. Xia, Q. et al. The type 2 diabetes presumed causal variant within TCF7L2 resides in an element that controls the expression of ACSL5. Diabetologia 59, 2360-2368 (2016).
4. Tsokos, G. C., Lo, M. S., Reis, P. &. Sullivan, K. E. New insights into the immunopathogenesis of systemic lupus erythematosus. Nat Rev Rheumatol 12, 716 730 (2016).
5. Song, W. & Craft, J. T follicular helper cell heterogeneity: Time, space, and function. Immunol Rev 288, 85-96 (2019).
6. Crotty, S. Follicular Helper CD4 T Cells (TFH). Annu Rev Immunol 29, 621-663 (2011).
7. Bentham, J. et al. Genetic association analyses implicate aberrant regulation of innate and adaptive immunity genes in the pathogenesis of systemic lupus erythematosus. Nat Genet 47, 1457 1464 (2015).
8. Morris, D. L. et al. Genome-wide association meta-analysis in Chinese and European individuals identifies ten new loci associated with systemic lupus erythematosus. Nat Genet 48, 940 946 (2016).
9. Thurman, R. E. et al. The accessible chromatin landscape of the human genome. Nature 489, 75 (2012).
10. Romberg, N. et al. CVID-associated TACT mutations affect autoreactive B cell selection and activation. J Clin Invest 123, 4283-4293 (2013).
11. Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat Methods 10, 1213 1218 (2013).
12. Adorini, L. & Penna, G. Control of autoimmune diseases by the vitamin D endocrine system. Nat Clin Pract Rheum 4, 404 EP-412 (2008).
13. Hughes, J. R. et al, Analysis of hundreds of cis-regulatory landscapes at high resolution in a single, high-throughput experiment. Nat Genet 46, 205 212 (2014).
14. Chesi, A. et al. Genome-scale Capture C promoter interactions implicate effector genes at GWAS loci for bone mineral density. Nat Commun 10, 1260 (2019).
15. Wingett, S. et al. HiCUP: pipeline for mapping and processing Hi-C data. F1000research 4, 1310 (2015).
16. Cairns, J. et al. CHiCAGO: robust detection of DNA looping interactions in Capture Hi-C data. Genome Biol 17, 127 (2016).
17. Javierre, B. M. et al. Lineage-Specific Genome Architecture Links Enhancers and Non-coding Disease Variants to Target Gene Promoters. Cell 167, 1369 1384.e19 (2016).
18. Ernst, J. et al. Mapping and analysis of chromatin state dynamics in nine human cell types. Nature 473, 43 49 (2011).
19. Weinstein, J. S. et al. Global transcriptome analysis and enhancer landscape of human primary T follicular helper and T effector lymphocytes. Blood 124, 3719 3729 (2014).
20. Bernstein, B. E. et al. The NIH Roadmap Epigenomics Mapping Consortium. Nat Biotechnol 28, 1045 (2010).
21. Cuddapah, S. et al. Global analysis of the insulator binding protein CTCF in chromatin barrier regions reveals demarcation of active and repressive domains. Genome Res 19, 24-32 (2009).
22. Johnston, R. J. et al. Bcl6 and Blimp-1 Are Reciprocal and Antagonistic Regulators of T Follicular Helper Cell Differentiation. Science 325, 1006-1010 (2009).
23. Kroenke, M. A. et al. Bcl6 and Maf Cooperate To Instruct Human Follicular Helper CD4 T Cell Differentiation. J Immunol 188, 3734-3744 (2012).
24. Hatzi, K. et al. BCL6 orchestrates Tfh cell differentiation via multiple distinct mechanisms. J Exp Medicine 212, 539-553 (2015).
25. Liu, X. et al. Genome-wide Analysis Identifies Bcl6-Controlled Regulatory Networks during T Follicular Helper Cell Differentiation. Cell Reports 14, 1735 1747 (2016).
26. Liu, X. et al. Bcl6 expression specifies the T follicular helper cell program in vivo. J Exp Medicine 209, 1841-1852 (2012).
27. Eystathioy, T., Peebles, C. L., Hamel, J. C., Vaughn, J. H. & Chan, E. K. Autoantibody to hLSm4 and the heptameric LSm complex in anti-Sm sera. Arthritis Rheumatism 46, 726-734 (2002).
28. Gunnewiek, K. J., van de Putte, L. & van Venrooij, W. The U1 snRNP complex: an autoantigen in connective tissue diseases. An update. Clin Exp Rheumatol 15, 549-60 (1997).
29. Odhams, C. A. et al. Mapping eQTLs with RNA-seq reveals novel susceptibility genes, non-coding RNAs and alternative-splicing events in systemic lupus erythematosus. Hum Mol Genet 26, 1003 1017 (2017).
30. Kim, Y., Choi, C., Lee, S., Conti, M. & Kim, Y. Homeodomain-interacting protein kinases, a novel family of co-repressors for homeodomain transcription factors. J Biol Chem 273, 25875 25879 (1998).
31. Nicke, B. et al. Involvement of MINK, a Ste20 Family Kinase, in Ras Oncogene-Induced Growth Arrest in Human Ovarian Surface Epithelial Cells. Mol Cell 20, 673-685 (2005).
32. Locci, M. et al. Activin A programs the differentiation of human TFH cells. Nat Immunol 17, 976 984 (2016).
33. Rubin, A. J. et al. Lineage-specific dynamic and pre-established enhancer-promoter contacts cooperate in terminal differentiation. Nat Genet 49, (2017).
34. Sanyal, A., Lajoie, B. R., Jain, G. & Dekker, J. The long-range interaction landscape of gene promoters. Nature 489, 109 (2012).
35. Bunting, K. L. et al. Multi-tiered Reorganization of the Genome during B Cell Affinity Maturation Anchored by a Germinal Center-Specific Locus Control Region. Immunity 45, 497 512 (2016).
36. Kondo, S. et al. Characterization of cells and gene-targeted mice deficient for the p53-binding kinase homeodomain-interacting protein kinase 1 (HIPK1). Proc National Acad Sci 100, 5431 5436 (2003).
37. Louie, S. H. et al. Modulation of the beta-catenin signaling pathway by the dishevelled-associated protein Hipk1. Plos One 4, e4310 (2009).
38. Shang, Y. et al. Transcriptional corepressors HIPK1 and HIPK2 control angiogenesis via TGF-β-TAK1-dependent mechanism. Plos Biol 11, e1001527 (2013).
39. Guerra, F. M., Gommerman, J. L., Corfe, S. A., Paige, C. J. & Rottapel, R. Homeodomain-Interacting Protein Kinase (HIPK)-1 Is Required for Splenic B Cell Homeostasis and Optimal T-independent Type 2 Humoral Response. Plos One 7, e35533 (2012).
40. Larhammar, M., Huntwork-Rodriguez, S., Rudhard, Y., Sengupta-Ghosh, A. & Lewcock, J. W. The Ste20 Family Kinases MAP4K4, MINK1, and TNIK Converge to Regulate Stress-induced JNK Signaling in Neurons. J Neurosci 37, 11074-11084 (2017).
41. Kaneko, S. et al. Smad inhibition by the Ste20 kinase Misshapen. Proc National Acad Sci 108, 11127-11132 (2011).
42. Fu, G. et al. Suppression of Th17 cell differentiation by misshapen/NIK-related kinase MINK1. J Exp Med 214, jem.20161120 (2017).
43. Ozaki, K. et al. A Critical Role for IL-21 in Regulating Immunoglobulin Production. Science 298, 1630-1634 (2002).
44. Sheffield, N. C. & Bock, C. LOLA: enrichment analysis for genomic region sets and regulatory elements in R and Bioconductor. Bioinformatics 32, 587-589 (2016).

TABLE 1
SLE variant-to-gene mapping using promoter-OCR
connectomes in follicular helper T cells.

| | | inferred GWAS | gene implicated by |
|---|---|---|---|
| proxySnp | sentinelSnp | gene | our 3D epigenomics |
| rs13356420 | rs6889239 | TNIP1 | ANXA6 |
| rs13356420 | rs6889239 | TNIP1 | TNIP1 |
| rs13356420 | rs6889239 | TNIP1 | DCTN4 |
| rs13356420 | rs6889239 | TNIP1 | ATOX1 |
| rs13356420 | rs6889239 | TNIP1 | G3BP1 |
| rs13356420 | rs6889239 | TNIP1 | GM2A |
| rs13356420 | rs6889239 | TNIP1 | CCDC69 |
| rs13356420 | rs10036748 | TNIP1 | ANXA6 |
| rs13356420 | rs10036748 | TNIP1 | TNIP1 |
| rs13356420 | rs10036748 | TNIP1 | DCTN4 |
| rs13356420 | rs10036748 | TNIP1 | ATOX1 |
| rs13356420 | rs10036748 | TNIP1 | G3BP1 |
| rs13356420 | rs10036748 | TNIP1 | GM2A |
| rs13356420 | rs10036748 | TNIP1 | CCDC69 |
| rs34053394 | rs12938617 | IKZF3 | IKZF3 |
| rs34053394 | rs12938617 | IKZF3 | IKZF3 |
| rs34053394 | rs2941509 | IKZF3 | IKZF3 |
| rs34053394 | rs2941509 | IKZF3 | IKZF3 |
| rs74625883 | rs268124 | SPRED2 | ACTR2 |
| rs74625883 | rs268124 | SPRED2 | SPRED2 |
| rs74625883 | rs268124 | SPRED2 | SPRED2 |
| rs74625883 | rs268124 | SPRED2 | SPRED2 |
| rs4416242 | rs28421442 | TET3 | AC073046.25 |
| rs4416242 | rs28421442 | TET3 | DGUOK |
| rs4416242 | rs28421442 | TET3 | RP11-711M9.1 |
| rs4416242 | rs6705628 | TET3 | AC073046.25 |
| rs4416242 | rs6705628 | TET3 | DGUOK |
| rs4416242 | rs6705628 | TET3 | RP11-711M9.1 |
| rs9915797 | rs12938617 | IKZF3 | IKZF3 |
| rs9915797 | rs2941509 | IKZF3 | IKZF3 |
| rs8071428 | rs34548580 | PLD2 | PFN1 |
| rs8071428 | rs34548580 | PLD2 | ENO3 |
| rs8071428 | rs34548580 | PLD2 | SLC25A11 |
| rs8071428 | rs34548580 | PLD2 | RNF167 |
| rs8071428 | rs34548580 | PLD2 | CHRNE |
| rs16843476 | rs34889541 | PTPRC | PTPRC |
| rs2004640 | rs3757387 | IRF5 | TSPAN33 |
| rs1051115 | rs9462027 | UHRF1BP1 | RP11-140K17.3 |
| rs1051115 | rs9462027 | UHRF1BP1 | ANKS1A |
| rs1051115 | rs9462027 | UHRF1BP1 | C6orf106 |
| rs1051115 | rs9462027 | UHRF1BP1 | TAF11 |
| rs1051115 | rs9462027 | UHRF1BP1 | SNRPC |
| rs1051115 | rs9462027 | UHRF1BP1 | C6orf1 |
| rs9912095 | rs12938617 | IKZF3 | IKZF3 |
| rs9912095 | rs2941509 | IKZF3 | IKZF3 |
| rs16843412 | rs34889541 | PTPRC | RP11-553K8.2 |
| rs16843412 | rs34889541 | PTPRC | PTPRC |
| rs148910232 | rs34330 | CDKN1B | LOH12CR2 |
| rs148910232 | rs34330 | CDKN1B | GPR19 |
| rs148910232 | rs34330 | CDKN1B | CDKN1B |
| rs148910232 | rs34330 | CDKN1B | DUSP16 |
| rs148910232 | rs34330 | CDKN1B | DUSP16 |
| rs148910232 | rs34330 | CDKN1B | TCONS_00021074 |
| rs148910232 | rs34330 | CDKN1B | MANSC1 |
| rs148910232 | rs34330 | CDKN1B | LOH12CR1 |
| rs148910232 | rs34330 | CDKN1B | TCONS_00020168 |
| rs142738614 | rs3757387 | IRF5 | TSPAN33 |
| rs17167484 | rs4388254 | TCF7 | SAR1B |
| rs17167484 | rs4388254 | TCF7 | SAR1B |
| rs17167484 | rs4388254 | TCF7 | CTD-2410N18.3 |
| rs17167484 | rs4388254 | TCF7 | VDAC1 |
| rs17167484 | rs4388254 | TCF7 | PPP2CA |
| rs17167484 | rs4388254 | TCF7 | UBE2B |
| rs17167484 | rs4388254 | TCF7 | CTB-113I20.2 |
| rs17167484 | rs4388254 | TCF7 | CDKN2AIPNL |
| rs17167484 | rs4388254 | TCF7 | PHF15 |
| rs17167484 | rs4388254 | TCF7 | PHF15 |
| rs17167484 | rs4388254 | TCF7 | PHF15 |
| rs17167484 | rs4388254 | TCF7 | PHF15 |
| rs17167484 | rs4388254 | TCF7 | MIR3661 |
| rs17167484 | rs4388254 | TCF7 | TXNDC15 |
| rs17167484 | rs4388254 | TCF7 | SEC24A |
| rs17167484 | rs4388254 | TCF7 | CTD-2410N18.5 |
| rs17167484 | rs4388254 | TCF7 | AC005355.1 |
| rs17167484 | rs4388254 | TCF7 | CAMLG |
| rs17167484 | rs4388254 | TCF7 | CDKL3 |
| rs17167484 | rs4388254 | TCF7 | CDKL3 |
| rs17167484 | rs4388254 | TCF7 | SKP1 |
| rs17167484 | rs4388254 | TCF7 | DDX46 |
| rs17167484 | rs4388254 | TCF7 | TCF7 |
| rs17167484 | rs4388254 | TCF7 | TCF7 |
| rs7768653 | rs7768653 | PRDM1 | RP1-134E15.3 |
| rs7768653 | rs7768653 | PRDM1 | PRDM1 |
| rs7768653 | rs7768653 | PRDM1 | PRDM1 |
| rs7768653 | rs6568431 | PRDM1 | RP1-134E15.3 |
| rs7768653 | rs6568431 | PRDM1 | PRDM1 |
| rs7768653 | rs6568431 | PRDM1 | PRDM1 |
| rs71406847 | rs28421442 | TET3 | AC073046.25 |
| rs71406847 | rs28421442 | TET3 | DGUOK |
| rs71406847 | rs28421442 | TET3 | RP11-711M9.1 |
| rs71406847 | rs6705628 | TET3 | AC073046.25 |
| rs71406847 | rs6705628 | TET3 | DGUOK |
| rs71406847 | rs6705628 | TET3 | RP11-711M9.1 |
| rs7205474 | rs9652601 | CIITA | RP11-485G7.5 |
| rs7205474 | rs9652601 | CIITA | RMI2 |
| rs3117582 | rs1150757 | MHC | NFKBIL1 |
| rs3117582 | rs1150757 | MHC | CLIC1 |
| rs3117582 | rs1150757 | MHC | PRRC2A |
| rs3117582 | rs1150757 | MHC | C6orf47 |
| rs3117582 | rs1150757 | MHC | ATP6V1G2-DDX39B |
| rs3117582 | rs1150757 | MHC | SNORD48 |
| rs3117582 | rs1150757 | MHC | GPANK1 |
| rs3117582 | rs1150757 | MHC | GPANK1 |
| rs3117582 | rs1150757 | MHC | C2 |
| rs3117582 | rs1150757 | MHC | C2 |
| rs3117582 | rs1150757 | MHC | CSNK2B-LY6G5B-1181 |
| rs3117582 | rs1150757 | MHC | CSNK2B-LY6G5B-1181 |
| rs3117582 | rs1150757 | MHC | SNORD84 |
| rs3117582 | rs1150757 | MHC | DDX39B-AS1 |
| rs3117582 | rs1150757 | MHC | ABHD16A |
| rs3117582 | rs1150757 | MHC | ATP6V1G2 |
| rs3117582 | rs1150757 | MHC | C6orf48 |
| rs3117582 | rs1150757 | MHC | DDX39B |
| rs3117582 | rs1150757 | MHC | CSNK2B |
| rs3117582 | rs1150757 | MHC | CSNK2B |
| rs3117582 | rs1150757 | MHC | HSPA1L |
| rs3117582 | rs1150757 | MHC | EHMT2 |
| rs3117582 | rs1150757 | MHC | ZBTB12 |
| rs3117582 | rs1150757 | MHC | LSM2 |
| rs3117582 | rs1150757 | MHC | TNF |
| rs3117582 | rs1150757 | MHC | HSPA1A |
| rs4681842 | rs9311676 | ABHD6 | TCONS_00006035 |
| rs4681842 | rs9311676 | ABHD6 | PDHB |
| rs4681842 | rs9311676 | ABHD6 | RP11-359I18.5 |
| rs4681842 | rs9311676 | ABHD6 | TCONS_00006034 |
| rs4681842 | rs9311676 | ABHD6 | KCTD6 |
| rs4681842 | rs9311676 | ABHD6 | TCONS_00006864 |
| rs71368521 | rs2286672 | PLD2 | MINK1 |
| rs4951985 | rs7579944 | LBH | LCLAT1 |
| rs6908065 | rs1150757 | MHC | HLA-DRA |
| rs13240595 | rs35000415 | IRF5 | IRF5 |
| rs2272165 | rs28421442 | TET3 | AC073046.25 |
| rs2272165 | rs28421442 | TET3 | TCONS_00002707 |
| rs2272165 | rs28421442 | TET3 | MTHFD2 |
| rs2272165 | rs28421442 | TET3 | TCONS_00002708 |
| rs2272165 | rs28421442 | TET3 | DUSP11 |
| rs2272165 | rs28421442 | TET3 | DGUOK |
| rs2272165 | rs28421442 | TET3 | BOLA3-AS1 |
| rs2272165 | rs28421442 | TET3 | TCONS_00002706 |
| rs2272165 | rs28421442 | TET3 | BOLA3 |
| rs2272165 | rs28421442 | TET3 | RP11-711M9.1 |
| rs2272165 | rs28421442 | TET3 | TCONS_00003734 |
| rs2272165 | rs28421442 | TET3 | RTKN |
| rs2272165 | rs28421442 | TET3 | TCONS_00003733 |
| rs2272165 | rs28421442 | TET3 | STAMBP |
| rs2272165 | rs28421442 | TET3 | TET3 |
| rs2272165 | rs6705628 | TET3 | AC073046.25 |
| rs2272165 | rs6705628 | TET3 | TCONS_00002707 |
| rs2272165 | rs6705628 | TET3 | MTHFD2 |
| rs2272165 | rs6705628 | TET3 | TCONS_00002708 |
| rs2272165 | rs6705628 | TET3 | DUSP11 |
| rs2272165 | rs6705628 | TET3 | DGUOK |
| rs2272165 | rs6705628 | TET3 | BOLA3-AS1 |
| rs2272165 | rs6705628 | TET3 | TCONS_00002706 |
| rs2272165 | rs6705628 | TET3 | BOLA3 |
| rs2272165 | rs6705628 | TET3 | RP11-711M9.1 |
| rs2272165 | rs6705628 | TET3 | TCONS_00003734 |
| rs2272165 | rs6705628 | TET3 | RTKN |
| rs2272165 | rs6705628 | TET3 | TCONS_00003733 |
| rs2272165 | rs6705628 | TET3 | STAMBP |
| rs2272165 | rs6705628 | TET3 | TET3 |
| rs28537796 | rs12938617 | IKZF3 | IKZF3 |
| rs28537796 | rs12938617 | IKZF3 | IKZF3 |
| rs28537796 | rs2941509 | IKZF3 | IKZF3 |
| rs28537796 | rs2941509 | IKZF3 | IKZF3 |
| rs12146493 | rs494003 | RNASEH2C | LTBP3 |
| rs12146493 | rs494003 | RNASEH2C | FIBP |
| rs12146493 | rs494003 | RNASEH2C | EIF1AD |
| rs12146493 | rs494003 | RNASEH2C | RP11-770G2.4 |
| rs12146493 | rs494003 | RNASEH2C | TCONS_I2_00005065 |
| rs12146493 | rs494003 | RNASEH2C | BANF1 |
| rs12146493 | rs494003 | RNASEH2C | TCONS_I2_00005064 |
| rs12146493 | rs494003 | RNASEH2C | SNX32 |
| rs12946040 | rs12938617 | IKZF3 | IKZF3 |
| rs12946040 | rs12938617 | IKZF3 | IKZF3 |
| rs12946040 | rs12938617 | IKZF3 | ZPBP2 |
| rs12946040 | rs2941509 | IKZF3 | IKZF3 |
| rs12946040 | rs2941509 | IKZF3 | IKZF3 |
| rs12946040 | rs2941509 | IKZF3 | ZPBP2 |
| rs12943633 | rs12938617 | IKZF3 | IKZF3 |
| rs12943633 | rs12938617 | IKZF3 | IKZF3 |
| rs12943633 | rs2941509 | IKZF3 | IKZF3 |
| rs12943633 | rs2941509 | IKZF3 | IKZF3 |
| rs1170443 | rs1170426 | ZFP90 | CHTF8 |
| rs1170443 | rs1170426 | ZFP90 | CIRH1A |
| rs7188928 | rs9652601 | CIITA | RP11-490O6.2 |
| rs7188928 | rs9652601 | CIITA | RP11-485G7.5 |
| rs7188928 | rs9652601 | CIITA | SOCS1 |
| rs7188928 | rs9652601 | CIITA | TXNDC11 |
| rs7188928 | rs9652601 | CIITA | RMI2 |
| rs1728769 | rs1170426 | ZFP90 | CHTF8 |
| rs1728769 | rs1170426 | ZFP90 | CIRH1A |
| rs1510475 | rs12938617 | IKZF3 | IKZF3 |
| rs1510475 | rs12938617 | IKZF3 | ZPBP2 |
| rs1510475 | rs2941509 | IKZF3 | IKZF3 |
| rs1510475 | rs2941509 | IKZF3 | ZPBP2 |
| rs7769961 | rs9462027 | UHRF1BP1 | RP11-140K17.3 |
| rs7769961 | rs9462027 | UHRF1BP1 | C6orf106 |
| rs7769961 | rs9462027 | UHRF1BP1 | SNRPC |
| rs1379247 | rs4388254 | TCF7 | SAR1B |
| rs1379247 | rs4388254 | TCF7 | SAR1B |
| rs1379247 | rs4388254 | TCF7 | CTD-2410N18.3 |
| rs1379247 | rs4388254 | TCF7 | VDAC1 |
| rs1379247 | rs4388254 | TCF7 | PPP2CA |
| rs1379247 | rs4388254 | TCF7 | UBE2B |
| rs1379247 | rs4388254 | TCF7 | CTB-113I20.2 |
| rs1379247 | rs4388254 | TCF7 | CDKN2AIPNL |
| rs1379247 | rs4388254 | TCF7 | PHF15 |
| rs1379247 | rs4388254 | TCF7 | PHF15 |
| rs1379247 | rs4388254 | TCF7 | PHF15 |
| rs1379247 | rs4388254 | TCF7 | PHF15 |
| rs1379247 | rs4388254 | TCF7 | MIR3661 |
| rs1379247 | rs4388254 | TCF7 | TXNDC15 |
| rs1379247 | rs4388254 | TCF7 | SEC24A |
| rs1379247 | rs4388254 | TCF7 | CTD-2410N18.5 |
| rs1379247 | rs4388254 | TCF7 | AC005355.1 |
| rs1379247 | rs4388254 | TCF7 | CAMLG |
| rs1379247 | rs4388254 | TCF7 | CDKL3 |
| rs1379247 | rs4388254 | TCF7 | CDKL3 |
| rs1379247 | rs4388254 | TCF7 | SKP1 |
| rs1379247 | rs4388254 | TCF7 | DDX46 |
| rs1379247 | rs4388254 | TCF7 | TCF7 |
| rs1379247 | rs4388254 | TCF7 | TCF7 |
| rs7944860 | rs494003 | RNASEH2C | LTBP3 |
| rs7944860 | rs494003 | RNASEH2C | DRAP1 |
| rs7944860 | rs494003 | RNASEH2C | PCNXL3 |
| rs7944860 | rs494003 | RNASEH2C | MAP3K11 |
| rs7944860 | rs494003 | RNASEH2C | EIF1AD |
| rs7944860 | rs494003 | RNASEH2C | MUS81 |
| rs7944860 | rs494003 | RNASEH2C | SIPA1 |
| rs7944860 | rs494003 | RNASEH2C | TCONS_I2_00005065 |
| rs7944860 | rs494003 | RNASEH2C | SNX32 |
| rs7944860 | rs494003 | RNASEH2C | SCYL1 |
| rs7944860 | rs494003 | RNASEH2C | C11orf68 |
| rs7944860 | rs494003 | RNASEH2C | CFL1 |
| rs7944860 | rs494003 | RNASEH2C | CFL1 |
| rs7944860 | rs494003 | RNASEH2C | FAM89B |
| rs7944860 | rs494003 | RNASEH2C | BANF1 |
| rs7944860 | rs494003 | RNASEH2C | EHBP1L1 |
| rs7944860 | rs494003 | RNASEH2C | EHBP1L1 |
| rs7944860 | rs494003 | RNASEH2C | TCONS_I2_00005064 |
| rs1014707 | rs34330 | CDKN1B | DUSP16 |
| rs1014707 | rs34330 | CDKN1B | DUSP16 |
| rs10175798 | rs7579944 | LBH | TCONS_00003622 |
| rs10175798 | rs7579944 | LBH | LCLAT1 |
| rs10175798 | rs7579944 | LBH | TCONS_00004194 |
| rs10175798 | rs7579944 | LBH | TCONS_00004193 |
| rs10175798 | rs7579944 | LBH | TCONS_00005128 |
| rs1538633 | rs1538633 | IL28RA | NIPAL3 |
| rs1538633 | rs1538633 | IL28RA | STPG1 |
| rs1538633 | rs4649203 | IL28RA | NIPAL3 |
| rs1538633 | rs4649203 | IL28RA | STPG1 |
| rs2001100 | rs1150757 | MHC | HLA-DRA |
| rs9268534 | rs1150757 | MHC | HLA-DRA |
| rs112324823 | rs9652601 | CIITA | RP11-485G7.5 |
| rs112324823 | rs9652601 | CIITA | RMI2 |
| rs2395599 | rs9462027 | UHRF1BP1 | RP11-140K17.3 |
| rs2395599 | rs9462027 | UHRF1BP1 | C6orf106 |
| rs78377363 | rs77000060 | TNFAIP3 | TCONS_00012608 |
| rs78377363 | rs77000060 | TNFAIP3 | TCONS_00012609 |
| rs78377363 | rs77000060 | TNFAIP3 | TNFAIP3 |
| rs78377363 | rs77000060 | TNFAIP3 | RP11-356I2.4 |
| rs78377363 | rs77000060 | TNFAIP3 | RP11-240M16.1 |
| rs78377363 | rs77000060 | TNFAIP3 | RP11-10J5.1 |
| rs78377363 | rs2230926 | TNFAIP3 | TCONS_00012608 |
| rs78377363 | rs2230926 | TNFAIP3 | TCONS_00012609 |
| rs78377363 | rs2230926 | TNFAIP3 | TNFAIP3 |
| rs78377363 | rs2230926 | TNFAIP3 | RP11-356I2.4 |
| rs78377363 | rs2230926 | TNFAIP3 | RP11-240M16.1 |
| rs78377363 | rs2230926 | TNFAIP3 | RP11-10J5.1 |
| rs4843867 | rs13332649 | IRF8 | IRF8 |
| rs4843867 | rs13332649 | IRF8 | TCONS_00024489 |
| rs2274365 | rs34889541 | PTPRC | MIR181A1HG |
| rs2274365 | rs34889541 | PTPRC | TCONS_00000111 |
| rs2274365 | rs34889541 | PTPRC | RP11-553K8.2 |
| rs2274365 | rs34889541 | PTPRC | NEK7 |
| rs7568254 | rs7579944 | LBH | LCLAT1 |
| rs113333331 | rs4649203 | IL28RA | NIPAL3 |
| rs113333331 | rs4649203 | IL28RA | STPG1 |
| rs2110585 | rs4388254 | TCF7 | SEC24A |
| rs2110585 | rs4388254 | TCF7 | SAR1B |
| rs2110585 | rs4388254 | TCF7 | CTD-2410N18.5 |
| rs2110585 | rs4388254 | TCF7 | AC005355.1 |
| rs2110585 | rs4388254 | TCF7 | CDKL3 |
| rs2110585 | rs4388254 | TCF7 | CDKL3 |
| rs2110585 | rs4388254 | TCF7 | CTD-2410N18.3 |
| rs2110585 | rs4388254 | TCF7 | PPP2CA |
| rs2110585 | rs4388254 | TCF7 | CDKN2AIPNL |
| rs2110585 | rs4388254 | TCF7 | UBE2B |
| rs2110585 | rs4388254 | TCF7 | MIR3661 |
| rs7105899 | rs7941765 | ETS1 | RP11-1007G5.2 |
| rs7105899 | rs7941765 | ETS1 | TCONS_00019532 |
| rs7105899 | rs7941765 | ETS1 | ETS1 |
| rs34004859 | rs268124 | SPRED2 | ACTR2 |
| rs34004859 | rs268124 | SPRED2 | SPRED2 |
| rs34004859 | rs268124 | SPRED2 | SPRED2 |
| rs34004859 | rs268124 | SPRED2 | SPRED2 |
| rs11347703 | rs13332649 | IRF8 | IRF8 |
| rs11347703 | rs13332649 | IRF8 | TCONS_00024489 |
| rs10896027 | rs494003 | RNASEH2C | FIBP |
| rs10896027 | rs494003 | RNASEH2C | MUS81 |
| rs10896027 | rs494003 | RNASEH2C | MUS81 |
| rs10896027 | rs494003 | RNASEH2C | MUS81 |
| rs10896027 | rs494003 | RNASEH2C | RELA |
| rs10896027 | rs494003 | RNASEH2C | CFL1 |
| rs10896027 | rs494003 | RNASEH2C | CFL1 |
| rs10896027 | rs494003 | RNASEH2C | CFL1 |
| rs10896027 | rs494003 | RNASEH2C | CFL1 |
| rs6900426 | rs9462027 | UHRF1BP1 | RP11-140K17.3 |
| rs6900426 | rs9462027 | UHRF1BP1 | C6orf106 |
| rs1862364 | rs6889239 | TNIP1 | TNIP1 |
| rs1862364 | rs6889239 | TNIP1 | TNIP1 |
| rs1862364 | rs10036748 | TNIP1 | TNIP1 |
| rs1862364 | rs10036748 | TNIP1 | TNIP1 |
| rs13386455 | rs17321999 | LBH | LCLAT1 |
| rs8059144 | rs13332649 | IRF8 | IRF8 |
| rs8059144 | rs13332649 | IRF8 | TCONS_00024489 |
| rs10803116 | rs9782955 | LYST | B3GALNT2 |
| rs10803116 | rs9782955 | LYST | LYST |
| rs3130476 | rs1150757 | MHC | CLIC1 |
| rs3130476 | rs1150757 | MHC | CLIC1 |
| rs3130476 | rs1150757 | MHC | NFKBIL1 |
| rs3130476 | rs1150757 | MHC | NFKBIL1 |
| rs3130476 | rs1150757 | MHC | DDAH2 |
| rs3130476 | rs1150757 | MHC | LST1 |
| rs3130476 | rs1150757 | MHC | PRRC2A |
| rs3130476 | rs1150757 | MHC | APOM |
| rs3130476 | rs1150757 | MHC | ATP6V1G2-DDX39B |
| rs3130476 | rs1150757 | MHC | ATP6V1G2-DDX39B |
| rs3130476 | rs1150757 | MHC | GPANK1 |
| rs3130476 | rs1150757 | MHC | GPANK1 |
| rs3130476 | rs1150757 | MHC | C2 |
| rs3130476 | rs1150757 | MHC | NEU1 |
| rs3130476 | rs1150757 | MHC | CSNK2B-LY6G5B-1181 |
| rs3130476 | rs1150757 | MHC | CSNK2B-LY6G5B-1181 |
| rs3130476 | rs1150757 | MHC | SNORD84 |
| rs3130476 | rs1150757 | MHC | DDX39B-AS1 |
| rs3130476 | rs1150757 | MHC | ABHD16A |
| rs3130476 | rs1150757 | MHC | LTB |
| rs3130476 | rs1150757 | MHC | LY6G5C |
| rs3130476 | rs1150757 | MHC | ATP6V1G2 |
| rs3130476 | rs1150757 | MHC | ATP6V1G2 |
| rs3130476 | rs1150757 | MHC | LTA |
| rs3130476 | rs1150757 | MHC | MSH5-SAPCD1 |
| rs3130476 | rs1150757 | MHC | BAG6 |
| rs3130476 | rs1150757 | MHC | DDX39B |
| rs3130476 | rs1150757 | MHC | MSH5 |
| rs3130476 | rs1150757 | MHC | CSNK2B |
| rs3130476 | rs1150757 | MHC | CSNK2B |
| rs3130476 | rs1150757 | MHC | EHMT2 |
| rs3130476 | rs1150757 | MHC | TNF |
| rs3794329 | rs17630801 | ELF1 | MTRF1 |
| rs3794329 | rs17630801 | ELF1 | KBTBD7 |
| rs3794329 | rs17630801 | ELF1 | ELF1 |
| rs3794329 | rs17630801 | ELF1 | ELF1 |
| rs3794329 | rs17630801 | ELF1 | NAA16 |
| rs3794329 | rs7329174 | ELF1 | MTRF1 |
| rs3794329 | rs7329174 | ELF1 | KBTBD7 |
| rs3794329 | rs7329174 | ELF1 | ELF1 |
| rs3794329 | rs7329174 | ELF1 | ELF1 |
| rs3794329 | rs7329174 | ELF1 | NAA16 |
| rs9901917 | rs12938617 | IKZF3 | IKZF3 |
| rs9901917 | rs12938617 | IKZF3 | IKZF3 |
| rs9901917 | rs2941509 | IKZF3 | IKZF3 |
| rs9901917 | rs2941509 | IKZF3 | IKZF3 |
| rs55780420 | rs4388254 | TCF7 | SAR1B |
| rs55780420 | rs4388254 | TCF7 | SEC24A |
| rs55780420 | rs4388254 | TCF7 | CTD-2410N18.5 |
| rs55780420 | rs4388254 | TCF7 | CDKL3 |
| rs55780420 | rs4388254 | TCF7 | VDAC1 |
| rs55780420 | rs4388254 | TCF7 | CTD-2410N18.3 |
| rs55780420 | rs4388254 | TCF7 | PPP2CA |
| rs55780420 | rs4388254 | TCF7 | CTB-113I20.2 |
| rs55780420 | rs4388254 | TCF7 | UBE2B |
| rs55780420 | rs4388254 | TCF7 | PHF15 |
| rs55780420 | rs4388254 | TCF7 | PHF15 |
| rs55780420 | rs4388254 | TCF7 | PHF15 |
| rs55780420 | rs4388254 | TCF7 | MIR3661 |
| rs55780420 | rs4388254 | TCF7 | TCF7 |
| rs55780420 | rs4388254 | TCF7 | TCF7 |
| rs112260932 | rs12938617 | IKZF3 | LRRC3C |
| rs112260932 | rs12938617 | IKZF3 | ZPBP2 |
| rs112260932 | rs2941509 | IKZF3 | LRRC3C |
| rs112260932 | rs2941509 | IKZF3 | ZPBP2 |
| rs11055030 | rs34330 | CDKN1B | LOH12CR2 |
| rs11055030 | rs34330 | CDKN1B | GPR19 |
| rs11055030 | rs34330 | CDKN1B | CDKN1B |
| rs11055030 | rs34330 | CDKN1B | DUSP16 |
| rs11055030 | rs34330 | CDKN1B | DUSP16 |
| rs11055030 | rs34330 | CDKN1B | TCONS_00021074 |
| rs11055030 | rs34330 | CDKN1B | MANSC1 |
| rs11055030 | rs34330 | CDKN1B | LOH12CR1 |
| rs11055030 | rs34330 | CDKN1B | TCONS_00020168 |
| rs7187539 | rs9652601 | CIITA | RP11-490O6.2 |
| rs7187539 | rs9652601 | CIITA | RP11-485G7.5 |
| rs7187539 | rs9652601 | CIITA | SOCS1 |
| rs7187539 | rs9652601 | CIITA | TXNDC11 |
| rs7187539 | rs9652601 | CIITA | RMI2 |
| rs7941765 | rs7941765 | ETS1 | RP11-1007G5.2 |
| rs7941765 | rs7941765 | ETS1 | TCONS_00019532 |
| rs7941765 | rs7941765 | ETS1 | ETS1 |
| rs28537207 | rs1170426 | ZFP90 | CHTF8 |
| rs28537207 | rs1170426 | ZFP90 | CIRH1A |
| rs3750997 | rs3794060 | DHCR7 | NADSYN1 |
| rs3750997 | rs3794060 | DHCR7 | DHCR7 |
| rs3750997 | rs7118246 | DHCR7 | NADSYN1 |
| rs3750997 | rs7118246 | DHCR7 | DHCR7 |
| rs2001099 | rs1150757 | MHC | HLA-DRA |
| rs112880843 | rs12938617 | IKZF3 | STARD3 |
| rs112880843 | rs12938617 | IKZF3 | RP11-387H17.4 |
| rs112880843 | rs12938617 | IKZF3 | PSMD3 |
| rs112880843 | rs12938617 | IKZF3 | GSDMB |
| rs112880843 | rs12938617 | IKZF3 | PGAP3 |
| rs112880843 | rs12938617 | IKZF3 | ERBB2 |
| rs112880843 | rs12938617 | IKZF3 | IKZF3 |
| rs112880843 | rs12938617 | IKZF3 | ORMDL3 |
| rs112880843 | rs12938617 | IKZF3 | ORMDL3 |
| rs112880843 | rs12938617 | IKZF3 | LRRC3C |
| rs112880843 | rs2941509 | IKZF3 | STARD3 |
| rs112880843 | rs2941509 | IKZF3 | RP11-387H17.4 |
| rs112880843 | rs2941509 | IKZF3 | PSMD3 |
| rs112880843 | rs2941509 | IKZF3 | GSDMB |
| rs112880843 | rs2941509 | IKZF3 | PGAP3 |
| rs112880843 | rs2941509 | IKZF3 | ERBB2 |
| rs112880843 | rs2941509 | IKZF3 | IKZF3 |
| rs112880843 | rs2941509 | IKZF3 | ORMDL3 |
| rs112880843 | rs2941509 | IKZF3 | ORMDL3 |
| rs112880843 | rs2941509 | IKZF3 | LRRC3C |
| rs238234 | rs34548580 | PLD2 | SLC25A11 |
| rs238234 | rs34548580 | PLD2 | RNF167 |
| rs35749047 | rs28421442 | TET3 | AC073046.25 |
| rs35749047 | rs28421442 | TET3 | RP11-711M9.1 |
| rs35749047 | rs6705628 | TET3 | AC073046.25 |
| rs35749047 | rs6705628 | TET3 | RP11-711M9.1 |
| rs78312791 | rs4649203 | IL28RA | NIPAL3 |
| rs78312791 | rs4649203 | IL28RA | STPG1 |
| rs78312791 | rs4649203 | IL28RA | STPG1 |
| rs78312791 | rs4649203 | IL28RA | STPG1 |
| rs78312791 | rs4649203 | IL28RA | STPG1 |
| rs1645935 | rs1170426 | ZFP90 | CHTF8 |
| rs1645935 | rs1170426 | ZFP90 | CIRH1A |
| rs35475765 | rs4388254 | TCF7 | PHF15 |
| rs35475765 | rs4388254 | TCF7 | PHF15 |
| rs35475765 | rs4388254 | TCF7 | PHF15 |
| rs35475765 | rs4388254 | TCF7 | TCF7 |
| rs35475765 | rs4388254 | TCF7 | TCF7 |
| rs35475765 | rs4388254 | TCF7 | AC005355.1 |
| rs906868 | rs7579944 | LBH | TCONS_00003622 |
| rs906868 | rs7579944 | LBH | LCLAT1 |
| rs906868 | rs7579944 | LBH | TCONS_00004194 |
| rs906868 | rs7579944 | LBH | TCONS_00004193 |
| rs906868 | rs7579944 | LBH | TCONS_00005128 |
| rs11719450 | rs9311676 | ABHD6 | RP11-359I18.5 |
| rs11719450 | rs9311676 | ABHD6 | KCTD6 |
| rs4944946 | rs3794060 | DHCR7 | NADSYN1 |
| rs4944946 | rs3794060 | DHCR7 | DHCR7 |
| rs4944946 | rs7118246 | DHCR7 | NADSYN1 |
| rs4944946 | rs7118246 | DHCR7 | DHCR7 |
| rs10525938 | rs9462027 | UHRF1BP1 | RP11-140K17.3 |
| rs10525938 | rs9462027 | UHRF1BP1 | C6orf106 |
| rs10525938 | rs9462027 | UHRF1BP1 | SNRPC |
| rs35520113 | rs268124 | SPRED2 | ACTR2 |
| rs35520113 | rs268124 | SPRED2 | AC074391.1 |
| rs35520113 | rs268124 | SPRED2 | SPRED2 |
| rs1170444 | rs1170426 | ZFP90 | CHTF8 |
| rs1170444 | rs1170426 | ZFP90 | CIRH1A |
| rs8045825 | rs13332649 | IRF8 | IRF8 |
| rs8045825 | rs13332649 | IRF8 | TCONS_00024489 |
| rs10036748 | rs6889239 | TNIP1 | TNIP1 |
| rs10036748 | rs10036748 | TNIP1 | TNIP1 |
| rs77013147 | rs12938617 | IKZF3 | STARD3 |
| rs77013147 | rs12938617 | IKZF3 | IKZF3 |
| rs77013147 | rs12938617 | IKZF3 | IKZF3 |
| rs77013147 | rs12938617 | IKZF3 | ZPBP2 |
| rs77013147 | rs2941509 | IKZF3 | STARD3 |
| rs77013147 | rs2941509 | IKZF3 | IKZF3 |
| rs77013147 | rs2941509 | IKZF3 | IKZF3 |
| rs77013147 | rs2941509 | IKZF3 | ZPBP2 |
| rs57680405 | rs34889541 | PTPRC | RP11-553K8.2 |
| rs57680405 | rs34889541 | PTPRC | PTPRC |
| rs7752060 | rs9462027 | UHRF1BP1 | RP11-140K17.3 |
| rs7752060 | rs9462027 | UHRF1BP1 | C6orf106 |
| rs7752060 | rs9462027 | UHRF1BP1 | SNRPC |
| rs35987776 | rs9462027 | UHRF1BP1 | RP11-140K17.3 |
| rs35987776 | rs9462027 | UHRF1BP1 | C6orf106 |
| rs12486509 | rs6762714 | LPP-TPRG1 | RP11-132N15.3 |
| rs12486509 | rs6762714 | LPP-TPRG1 | BCL6 |
| rs12486509 | rs6762714 | LPP-TPRG1 | BCL6 |
| rs17535158 | rs268124 | SPRED2 | AC074391.1 |
| rs17535158 | rs268124 | SPRED2 | SPRED2 |
| rs3178615 | rs4388254 | TCF7 | TCF7 |
| rs3178615 | rs4388254 | TCF7 | TCF7 |
| rs13006407 | rs28421442 | TET3 | AC073046.25 |
| rs13006407 | rs28421442 | TET3 | RP11-711M9.1 |
| rs13006407 | rs6705628 | TET3 | AC073046.25 |
| rs13006407 | rs6705628 | TET3 | RP11-711M9.1 |
| rs4783653 | rs1170426 | ZFP90 | CHTF8 |
| rs4783653 | rs1170426 | ZFP90 | CIRH1A |
| rs960709 | rs6889239 | TNIP1 | ANXA6 |
| rs960709 | rs6889239 | TNIP1 | TNIP1 |
| rs960709 | rs6889239 | TNIP1 | DCTN4 |
| rs960709 | rs6889239 | TNIP1 | ATOX1 |
| rs960709 | rs6889239 | TNIP1 | G3BP1 |
| rs960709 | rs6889239 | TNIP1 | GM2A |
| rs960709 | rs6889239 | TNIP1 | CCDC69 |
| rs960709 | rs10036748 | TNIP1 | ANXA6 |
| rs960709 | rs10036748 | TNIP1 | TNIP1 |
| rs960709 | rs10036748 | TNIP1 | DCTN4 |
| rs960709 | rs10036748 | TNIP1 | ATOX1 |
| rs960709 | rs10036748 | TNIP1 | G3BP1 |
| rs960709 | rs10036748 | TNIP1 | GM2A |
| rs960709 | rs10036748 | TNIP1 | CCDC69 |
| rs76404385 | rs61432431 | ETS1 | RP11-1007G5.2 |
| rs76404385 | rs61432431 | ETS1 | TCONS_00019532 |
| rs76404385 | rs61432431 | ETS1 | FLI1-AS1 |
| rs76404385 | rs61432431 | ETS1 | FLI1 |
| rs58075375 | rs12938617 | IKZF3 | IKZF3 |
| rs58075375 | rs12938617 | IKZF3 | IKZF3 |
| rs58075375 | rs2941509 | IKZF3 | IKZF3 |
| rs58075375 | rs2941509 | IKZF3 | IKZF3 |
| rs6658406 | rs9782955 | LYST | B3GALNT2 |
| rs6658406 | rs9782955 | LYST | LYST |
| rs34086582 | rs9311676 | ABHD6 | RP11-359I18.5 |
| rs34086582 | rs9311676 | ABHD6 | KCTD6 |
| rs34086582 | rs9311676 | ABHD6 | PXK |
| rs34086582 | rs9311676 | ABHD6 | RPP14 |
| rs35809595 | rs10761602 | ARID5B | RTKN2 |
| rs35809595 | rs10761602 | ARID5B | ARID5B |
| rs35809595 | rs10761602 | ARID5B | ARID5B |
| rs35809595 | rs10761602 | ARID5B | ARID5B |
| rs35809595 | rs4948496 | ARID5B | RTKN2 |
| rs35809595 | rs4948496 | ARID5B | ARID5B |
| rs35809595 | rs4948496 | ARID5B | ARID5B |
| rs35809595 | rs4948496 | ARID5B | ARID5B |
| rs2282171 | rs12494314 | TMEM39A | COX17 |
| rs2282171 | rs12494314 | TMEM39A | TIMMDC1 |
| rs2282171 | rs12494314 | TMEM39A | TMEM39A |
| rs2282171 | rs12494314 | TMEM39A | TMEM39A |
| rs2282171 | rs12494314 | TMEM39A | TCONS_00005429 |
| rs2282171 | rs12494314 | TMEM39A | CD80 |
| rs2282171 | rs12494314 | TMEM39A | RP11-18H7.1 |
| rs2282171 | rs12494314 | TMEM39A | TCONS_00006174 |
| rs2282171 | rs12494314 | TMEM39A | TCONS_00006171 |
| rs2282171 | rs12494314 | TMEM39A | POGLUT1 |
| rs2282171 | rs12494314 | TMEM39A | TCONS_00006173 |
| rs2282171 | rs12494314 | TMEM39A | TCONS_00006175 |
| rs2282171 | rs12494314 | TMEM39A | MAATS1 |
| rs2282171 | rs12494314 | TMEM39A | RP11-190C22.8 |
| rs2282171 | rs12494314 | TMEM39A | TCONS_00006172 |
| rs2282171 | rs12494314 | TMEM39A | GSK3B |
| rs2282171 | rs12494314 | TMEM39A | GTF2E1 |
| rs2282171 | rs12494314 | TMEM39A | TCONS_00006941 |
| rs2282171 | rs12494314 | TMEM39A | TCONS_00006170 |
| rs2282171 | rs12494314 | TMEM39A | RABL3 |
| rs2282171 | rs12494314 | TMEM39A | ARHGAP31 |
| rs2282171 | rs12494314 | TMEM39A | TCONS_00006940 |
| rs4930156 | rs494003 | RNASEH2C | LTBP3 |
| rs4930156 | rs494003 | RNASEH2C | DRAP1 |
| rs4930156 | rs494003 | RNASEH2C | PCNXL3 |
| rs4930156 | rs494003 | RNASEH2C | MAP3K11 |
| rs4930156 | rs494003 | RNASEH2C | EIF1AD |
| rs4930156 | rs494003 | RNASEH2C | MUS81 |
| rs4930156 | rs494003 | RNASEH2C | SIPA1 |
| rs4930156 | rs494003 | RNASEH2C | TCONS_I2_00005065 |
| rs4930156 | rs494003 | RNASEH2C | SNX32 |
| rs4930156 | rs494003 | RNASEH2C | SCYL1 |
| rs4930156 | rs494003 | RNASEH2C | C11orf68 |
| rs4930156 | rs494003 | RNASEH2C | CFL1 |
| rs4930156 | rs494003 | RNASEH2C | CFL1 |
| rs4930156 | rs494003 | RNASEH2C | FAM89B |
| rs4930156 | rs494003 | RNASEH2C | BANF1 |
| rs4930156 | rs494003 | RNASEH2C | EHBP1L1 |
| rs4930156 | rs494003 | RNASEH2C | EHBP1L1 |
| rs4930156 | rs494003 | RNASEH2C | TCONS_I2_00005064 |
| rs71041848 | rs4639966 | Intergenic | DPAGT1 |
| rs71041848 | rs4639966 | Intergenic | BCL9L |
| rs71041848 | rs4639966 | Intergenic | C2CD2L |
| rs71041848 | rs4639966 | Intergenic | DDX6 |
| rs71041848 | rs4639966 | Intergenic | DDX6 |
| rs71041848 | rs4639966 | Intergenic | TCONS_00020001 |
| rs71041848 | rs4639966 | Intergenic | CXCR5 |
| rs71041848 | rs4639966 | Intergenic | TCONS_00019492 |
| rs71041848 | rs4639966 | Intergenic | MIR4492 |
| rs73790149 | rs4388254 | TCF7 | PHF15 |
| rs73790149 | rs4388254 | TCF7 | PHF15 |
| rs73790149 | rs4388254 | TCF7 | PHF15 |
| rs73790149 | rs4388254 | TCF7 | TCF7 |
| rs73790149 | rs4388254 | TCF7 | TCF7 |
| rs73790149 | rs4388254 | TCF7 | AC005355.1 |
| rs752637 | rs3757387 | IRF5 | TSPAN33 |
| rs752637 | rs3757387 | IRF5 | CCDC136 |
| rs1425801 | rs387619 | CD44 | RP4-607I7.1 |
| rs1425801 | rs387619 | CD44 | CD44 |
| rs28410471 | rs1170426 | ZFP90 | CHTF8 |
| rs28410471 | rs1170426 | ZFP90 | CIRH1A |
| rs111655589 | rs12418883 | IRF7 | RNH1 |
| rs111655589 | rs12418883 | IRF7 | DEAF1 |
| rs111655589 | rs12418883 | IRF7 | EPS8L2 |
| rs111655589 | rs12418883 | IRF7 | TALDO1 |
| rs111655589 | rs1061502 | IRF7 | RNH1 |
| rs111655589 | rs1061502 | IRF7 | DEAF1 |
| rs111655589 | rs1061502 | IRF7 | EPS8L2 |
| rs111655589 | rs1061502 | IRF7 | TALDO1 |
| rs3024495 | rs3024505 | IL10 | FAIM3 |
| rs13389106 | rs17321999 | LBH | LCLAT1 |
| rs34631447 | rs6762714 | LPP-TPRG1 | RP11-132N15.3 |
| rs34631447 | rs6762714 | LPP-TPRG1 | BCL6 |
| rs34631447 | rs6762714 | LPP-TPRG1 | BCL6 |
| rs17534034 | rs268134 | SPRED2 | AC074391.1 |
| rs17534034 | rs268134 | SPRED2 | SPRED2 |
| rs17534034 | rs268134 | SPRED2 | SPRED2 |
| rs13385731 | rs13385731 | RASGRP3 | TCONS_I2_00013556 |
| rs13385731 | rs13385731 | RASGRP3 | FAM98A |
| rs13385731 | rs13385731 | RASGRP3 | RASGRP3 |
| rs71424182 | rs268124 | SPRED2 | ACTR2 |
| rs71424182 | rs268124 | SPRED2 | AC074391.1 |
| rs71424182 | rs268124 | SPRED2 | SPRED2 |
| rs9268530 | rs1150757 | MHC | HLA-DRA |
| rs244656 | rs244687 | TCF7 | SAR1B |
| rs244656 | rs244687 | TCF7 | SEC24A |
| rs244656 | rs244687 | TCF7 | CTD-2410N18.5 |
| rs244656 | rs244687 | TCF7 | CDKL3 |
| rs244656 | rs244687 | TCF7 | CTD-2410N18.3 |
| rs244656 | rs244687 | TCF7 | VDAC1 |
| rs244656 | rs244687 | TCF7 | PPP2CA |
| rs244656 | rs244687 | TCF7 | UBE2B |
| rs244656 | rs244687 | TCF7 | CTB-113I20.2 |
| rs244656 | rs244687 | TCF7 | PHF15 |
| rs244656 | rs244687 | TCF7 | PHF15 |
| rs244656 | rs244687 | TCF7 | PHF15 |
| rs244656 | rs244687 | TCF7 | MIR3661 |
| rs244656 | rs244687 | TCF7 | TCF7 |
| rs244656 | rs244687 | TCF7 | TCF7 |
| rs28495738 | rs4388254 | TCF7 | PHF15 |
| rs28495738 | rs4388254 | TCF7 | PHF15 |
| rs28495738 | rs4388254 | TCF7 | PHF15 |
| rs28495738 | rs4388254 | TCF7 | TCF7 |
| rs28495738 | rs4388254 | TCF7 | TCF7 |
| rs28495738 | rs4388254 | TCF7 | AC005355.1 |
| rs41272536 | rs17849501 | SMG7 | ARPC5 |
| rs41272536 | rs17849501 | SMG7 | RP1-127C7.6 |
| rs41272536 | rs17849501 | SMG7 | TSEN15 |
| rs41272536 | rs17849501 | SMG7 | APOBEC4 |
| rs41272536 | rs17849501 | SMG7 | APOBEC4 |
| rs41272536 | rs17849501 | SMG7 | RNASEL |
| rs41272536 | rs17849501 | SMG7 | DHX9 |
| rs41272536 | rs17849501 | SMG7 | NCF2 |
| rs41272536 | rs17849501 | SMG7 | RGL1 |
| rs1728770 | rs1170426 | ZFP90 | CHTF8 |
| rs1728770 | rs1170426 | ZFP90 | CIRH1A |
| rs146710425 | rs28421442 | TET3 | AC073046.25 |
| rs146710425 | rs28421442 | TET3 | TCONS_00002707 |
| rs146710425 | rs28421442 | TET3 | MTHFD2 |
| rs146710425 | rs28421442 | TET3 | TCONS_00002708 |
| rs146710425 | rs28421442 | TET3 | DUSP11 |
| rs146710425 | rs28421442 | TET3 | DGUOK |
| rs146710425 | rs28421442 | TET3 | BOLA3-AS1 |
| rs146710425 | rs28421442 | TET3 | TCONS_00002706 |
| rs146710425 | rs28421442 | TET3 | BOLA3 |
| rs146710425 | rs28421442 | TET3 | RP11-711M9.1 |
| rs146710425 | rs28421442 | TET3 | TCONS_00003734 |
| rs146710425 | rs28421442 | TET3 | RTKN |
| rs146710425 | rs28421442 | TET3 | TCONS_00003733 |
| rs146710425 | rs28421442 | TET3 | STAMBP |
| rs146710425 | rs28421442 | TET3 | TET3 |
| rs146710425 | rs6705628 | TET3 | AC073046.25 |
| rs146710425 | rs6705628 | TET3 | TCONS_00002707 |
| rs146710425 | rs6705628 | TET3 | MTHFD2 |
| rs146710425 | rs6705628 | TET3 | TCONS_00002708 |
| rs146710425 | rs6705628 | TET3 | DUSP11 |
| rs146710425 | rs6705628 | TET3 | DGUOK |
| rs146710425 | rs6705628 | TET3 | BOLA3-AS1 |
| rs146710425 | rs6705628 | TET3 | TCONS_00002706 |
| rs146710425 | rs6705628 | TET3 | BOLA3 |
| rs146710425 | rs6705628 | TET3 | RP11-711M9.1 |
| rs146710425 | rs6705628 | TET3 | TCONS_00003734 |
| rs146710425 | rs6705628 | TET3 | RTKN |
| rs146710425 | rs6705628 | TET3 | TCONS_00003733 |
| rs146710425 | rs6705628 | TET3 | STAMBP |
| rs146710425 | rs6705628 | TET3 | TET3 |
| rs34325 | rs34330 | CDKN1B | LOH12CR2 |
| rs34325 | rs34330 | CDKN1B | GPR19 |
| rs34325 | rs34330 | CDKN1B | CDKN1B |
| rs34325 | rs34330 | CDKN1B | DUSP16 |
| rs34325 | rs34330 | CDKN1B | DUSP16 |
| rs34325 | rs34330 | CDKN1B | TCONS_00021074 |
| rs34325 | rs34330 | CDKN1B | MANSC1 |
| rs34325 | rs34330 | CDKN1B | LOH12CR1 |
| rs34325 | rs34330 | CDKN1B | TCONS_00020168 |
| rs6908056 | rs1150757 | MHC | HLA-DRA |
| rs3169574 | rs12938617 | IKZF3 | LRRC3C |
| rs3169574 | rs12938617 | IKZF3 | ZPBP2 |
| rs3169574 | rs2941509 | IKZF3 | LRRC3C |
| rs3169574 | rs2941509 | IKZF3 | ZPBP2 |
| rs1645936 | rs1170426 | ZFP90 | CHTF8 |
| rs1645936 | rs1170426 | ZFP90 | CIRH1A |
| rs3135382 | rs1150757 | MHC | HLA-DRA |
| rs112677036 | rs12938617 | IKZF3 | PGAP3 |
| rs112677036 | rs12938617 | IKZF3 | ERBB2 |
| rs112677036 | rs12938617 | IKZF3 | IKZF3 |
| rs112677036 | rs12938617 | IKZF3 | IKZF3 |
| rs112677036 | rs2941509 | IKZF3 | PGAP3 |
| rs112677036 | rs2941509 | IKZF3 | ERBB2 |
| rs112677036 | rs2941509 | IKZF3 | IKZF3 |
| rs112677036 | rs2941509 | IKZF3 | IKZF3 |
| rs371707645 | rs1150757 | MHC | HLA-DQB1 |
| rs371707645 | rs1150757 | MHC | HLA-DOB |
| rs35105110 | rs12938617 | IKZF3 | IKZF3 |
| rs35105110 | rs12938617 | IKZF3 | IKZF3 |
| rs35105110 | rs2941509 | IKZF3 | IKZF3 |
| rs35105110 | rs2941509 | IKZF3 | IKZF3 |
| rs11606611 | rs3794060 | DHCR7 | TCONS_00019374 |
| rs11606611 | rs3794060 | DHCR7 | RP11-660L16.2 |
| rs11606611 | rs3794060 | DHCR7 | DHCR7 |
| rs11606611 | rs7118246 | DHCR7 | TCONS_00019374 |
| rs11606611 | rs7118246 | DHCR7 | RP11-660L16.2 |
| rs11606611 | rs7118246 | DHCR7 | DHCR7 |
| rs56319840 | rs4388254 | TCF7 | SAR1B |
| rs56319840 | rs4388254 | TCF7 | SEC24A |
| rs56319840 | rs4388254 | TCF7 | CTD-2410N18.5 |
| rs56319840 | rs4388254 | TCF7 | CDKL3 |
| rs56319840 | rs4388254 | TCF7 | VDAC1 |
| rs56319840 | rs4388254 | TCF7 | CTD-2410N18.3 |
| rs56319840 | rs4388254 | TCF7 | PPP2CA |
| rs56319840 | rs4388254 | TCF7 | CTB-113I20.2 |
| rs56319840 | rs4388254 | TCF7 | UBE2B |
| rs56319840 | rs4388254 | TCF7 | PHF15 |
| rs56319840 | rs4388254 | TCF7 | PHF15 |
| rs56319840 | rs4388254 | TCF7 | PHF15 |
| rs56319840 | rs4388254 | TCF7 | MIR3661 |
| rs56319840 | rs4388254 | TCF7 | TCF7 |
| rs56319840 | rs4388254 | TCF7 | TCF7 |
| rs16843559 | rs34889541 | PTPRC | MIR181A1HG |
| rs16843559 | rs34889541 | PTPRC | TCONS_00000111 |
| rs16843559 | rs34889541 | PTPRC | RP11-553K8.2 |
| rs16843559 | rs34889541 | PTPRC | NEK7 |
| rs13386353 | rs17321999 | LBH | LCLAT1 |
| rs3117579 | rs1150757 | MHC | VWA7 |
| rs3117579 | rs1150757 | MHC | SNORD48 |
| rs3117579 | rs1150757 | MHC | C2 |
| rs3117579 | rs1150757 | MHC | C2 |
| rs3117579 | rs1150757 | MHC | NELFE |
| rs3117579 | rs1150757 | MHC | ABHD16A |
| rs3117579 | rs1150757 | MHC | SKIV2L |
| rs3117579 | rs1150757 | MHC | C6orf48 |
| rs3117579 | rs1150757 | MHC | LSM2 |
| rs3117579 | rs1150757 | MHC | VARS |
| rs3117579 | rs1150757 | MHC | NFKBIL1 |
| rs3117579 | rs1150757 | MHC | CLIC1 |
| rs3117579 | rs1150757 | MHC | TCONS_00011779 |
| rs3117579 | rs1150757 | MHC | C6orf47 |
| rs3117579 | rs1150757 | MHC | PRRC2A |
| rs3117579 | rs1150757 | MHC | APOM |
| rs3117579 | rs1150757 | MHC | ATP6V1G2-DDX39B |
| rs3117579 | rs1150757 | MHC | NEU1 |
| rs3117579 | rs1150757 | MHC | SNORD84 |
| rs3117579 | rs1150757 | MHC | DDX39B-AS1 |
| rs3117579 | rs1150757 | MHC | ATP6V1G2 |
| rs3117579 | rs1150757 | MHC | BAG6 |
| rs3117579 | rs1150757 | MHC | DDX39B |
| rs3117579 | rs1150757 | MHC | HSPA1L |
| rs3117579 | rs1150757 | MHC | EHMT2 |
| rs3117579 | rs1150757 | MHC | ZBTB12 |
| rs3117579 | rs1150757 | MHC | HSPA1A |
| rs3117579 | rs1150757 | MHC | TNF |
| rs71136618 | rs12917716 | CIITA | RP11-485G7.5 |
| rs71136618 | rs12917716 | CIITA | RMI2 |
| rs71136618 | rs9652601 | CIITA | RP11-485G7.5 |
| rs71136618 | rs9652601 | CIITA | RMI2 |
| rs2274472 | rs1887428 | JAK2 | ERMP1 |
| rs2274472 | rs1887428 | JAK2 | CD274 |
| rs2274472 | rs1887428 | JAK2 | RCL1 |
| rs2274472 | rs1887428 | JAK2 | AK3 |
| rs2274472 | rs1887428 | JAK2 | CDC37L1 |
| rs2274472 | rs1887428 | JAK2 | RP11-6J24.6 |
| rs2274472 | rs1887428 | JAK2 | PPAPDC2 |
| rs9894370 | rs12938617 | IKZF3 | IKZF3 |
| rs9894370 | rs12938617 | IKZF3 | IKZF3 |
| rs9894370 | rs12938617 | IKZF3 | LRRC3C |
| rs9894370 | rs12938617 | IKZF3 | ZPBP2 |
| rs9894370 | rs2941509 | IKZF3 | IKZF3 |
| rs9894370 | rs2941509 | IKZF3 | IKZF3 |
| rs9894370 | rs2941509 | IKZF3 | LRRC3C |
| rs9894370 | rs2941509 | IKZF3 | ZPBP2 |
| rs12917716 | rs12917716 | CIITA | RP11-485G7.5 |
| rs12917716 | rs12917716 | CIITA | RMI2 |
| rs12917716 | rs9652601 | CIITA | RP11-485G7.5 |
| rs12917716 | rs9652601 | CIITA | RMI2 |
| rs113794548 | rs12418883 | IRF7 | DEAF1 |
| rs113794548 | rs12418883 | IRF7 | DEAF1 |
| rs113794548 | rs12418883 | IRF7 | HRAS |
| rs113794548 | rs12418883 | IRF7 | HRAS |
| rs113794548 | rs12418883 | IRF7 | AP006621.9 |
| rs113794548 | rs12418883 | IRF7 | AP006621.9 |
| rs113794548 | rs12418883 | IRF7 | RNH1 |
| rs113794548 | rs12418883 | IRF7 | EPS8L2 |
| rs113794548 | rs12418883 | IRF7 | EPS8L2 |
| rs113794548 | rs12418883 | IRF7 | EPS8L2 |
| rs113794548 | rs12418883 | IRF7 | PSMD13 |
| rs113794548 | rs12418883 | IRF7 | LRRC56 |
| rs113794548 | rs12418883 | IRF7 | LRRC56 |
| rs113794548 | rs12418883 | IRF7 | PIDD |
| rs113794548 | rs12418883 | IRF7 | TMEM80 |
| rs113794548 | rs12418883 | IRF7 | CEND1 |
| rs113794548 | rs12418883 | IRF7 | RASSF7 |
| rs113794548 | rs12418883 | IRF7 | SIRT3 |
| rs113794548 | rs12418883 | IRF7 | C11orf35 |
| rs113794548 | rs12418883 | IRF7 | IRF7 |
| rs113794548 | rs1061502 | IRF7 | DEAF1 |
| rs113794548 | rs1061502 | IRF7 | DEAF1 |
| rs113794548 | rs1061502 | IRF7 | HRAS |
| rs113794548 | rs1061502 | IRF7 | HRAS |
| rs113794548 | rs1061502 | IRF7 | AP006621.9 |
| rs113794548 | rs1061502 | IRF7 | AP006621.9 |
| rs113794548 | rs1061502 | IRF7 | RNH1 |
| rs113794548 | rs1061502 | IRF7 | EPS8L2 |
| rs113794548 | rs1061502 | IRF7 | EPS8L2 |
| rs113794548 | rs1061502 | IRF7 | EPS8L2 |
| rs113794548 | rs1061502 | IRF7 | PSMD13 |
| rs113794548 | rs1061502 | IRF7 | LRRC56 |
| rs113794548 | rs1061502 | IRF7 | LRRC56 |
| rs113794548 | rs1061502 | IRF7 | PIDD |
| rs113794548 | rs1061502 | IRF7 | TMEM80 |
| rs113794548 | rs1061502 | IRF7 | CEND1 |
| rs113794548 | rs1061502 | IRF7 | RASSF7 |
| rs113794548 | rs1061502 | IRF7 | SIRT3 |
| rs113794548 | rs1061502 | IRF7 | C11orf35 |
| rs113794548 | rs1061502 | IRF7 | IRF7 |
| rs6930571 | rs1150757 | MHC | HLA-DRA |
| rs3129966 | rs1150757 | MHC | HLA-DQB1 |
| rs3129966 | rs1150757 | MHC | HLA-DOB |
| rs28364617 | rs3794060 | DHCR7 | NADSYN1 |
| rs28364617 | rs3794060 | DHCR7 | DHCR7 |
| rs28364617 | rs7118246 | DHCR7 | NADSYN1 |
| rs28364617 | rs7118246 | DHCR7 | DHCR7 |
| rs57668933 | rs17630801 | ELF1 | ELF1 |
| rs57668933 | rs17630801 | ELF1 | ELF1 |
| rs57668933 | rs17630801 | ELF1 | WBP4 |
| rs57668933 | rs7329174 | ELF1 | ELF1 |
| rs57668933 | rs7329174 | ELF1 | ELF1 |
| rs57668933 | rs7329174 | ELF1 | WBP4 |
| rs3754098 | rs34889541 | PTPRC | RP11-553K8.2 |
| rs3754098 | rs34889541 | PTPRC | PTPRC |
| rs3895755 | rs4388254 | TCF7 | SAR1B |
| rs3895755 | rs4388254 | TCF7 | SAR1B |
| rs3895755 | rs4388254 | TCF7 | SEC24A |
| rs3895755 | rs4388254 | TCF7 | AC005355.1 |
| rs3895755 | rs4388254 | TCF7 | CDKL3 |
| rs3895755 | rs4388254 | TCF7 | UBE2B |
| rs3895755 | rs4388254 | TCF7 | SKP1 |
| rs3895755 | rs4388254 | TCF7 | PHF15 |
| rs3895755 | rs4388254 | TCF7 | PHF15 |
| rs3895755 | rs4388254 | TCF7 | PHF15 |
| rs3895755 | rs4388254 | TCF7 | DDX46 |
| rs3895755 | rs4388254 | TCF7 | TCF7 |
| rs3895755 | rs4388254 | TCF7 | TCF7 |
| rs35493230 | rs268124 | SPRED2 | ACTR2 |
| rs35493230 | rs268124 | SPRED2 | AC074391.1 |
| rs35493230 | rs268124 | SPRED2 | SPRED2 |
| rs77216612 | rs34330 | CDKN1B | LOH12CR2 |
| rs77216612 | rs34330 | CDKN1B | GPR19 |
| rs77216612 | rs34330 | CDKN1B | CDKN1B |
| rs77216612 | rs34330 | CDKN1B | DUSP16 |
| rs77216612 | rs34330 | CDKN1B | DUSP16 |
| rs77216612 | rs34330 | CDKN1B | TCONS_00021074 |
| rs77216612 | rs34330 | CDKN1B | MANSC1 |
| rs77216612 | rs34330 | CDKN1B | LOH12CR1 |
| rs77216612 | rs34330 | CDKN1B | TCONS_00020168 |
| rs28723033 | rs4388254 | TCF7 | UBE2B |
| rs28723033 | rs4388254 | TCF7 | PHF15 |
| rs28723033 | rs4388254 | TCF7 | PHF15 |
| rs28723033 | rs4388254 | TCF7 | PHF15 |
| rs28723033 | rs4388254 | TCF7 | TCF7 |
| rs28723033 | rs4388254 | TCF7 | TCF7 |
| rs28723033 | rs4388254 | TCF7 | CDKL3 |
| rs6495131 | rs2289583 | CSK | CLK3 |
| rs6495131 | rs2289583 | CSK | CLK3 |
| rs6495131 | rs2289583 | CSK | C15orf39 |
| rs6495131 | rs2289583 | CSK | SCAMP5 |
| rs6495131 | rs2289583 | CSK | MPI |
| rs6495131 | rs2289583 | CSK | CSK |
| rs6495131 | rs2289583 | CSK | CSK |
| rs6495131 | rs2289583 | CSK | CSK |
| rs6495131 | rs2289583 | CSK | CSK |
| rs6495131 | rs2289583 | CSK | EDC3 |
| rs6495131 | rs2289583 | CSK | EDC3 |
| rs6495131 | rs2289583 | CSK | MIR4513 |
| rs6495131 | rs2289583 | CSK | CPLX3 |
| rs6495131 | rs2289583 | CSK | UBL7-AS1 |
| rs6495131 | rs2289583 | CSK | CTD-3154N5.1 |
| rs6495131 | rs2289583 | CSK | ULK3 |
| rs6495131 | rs2289583 | CSK | FAM219B |
| rs6495131 | rs2289583 | CSK | FAM219B |
| rs6495131 | rs2289583 | CSK | UBL7 |
| rs6495131 | rs55655136 | CSK | CLK3 |
| rs6495131 | rs55655136 | CSK | CLK3 |
| rs6495131 | rs55655136 | CSK | C15orf39 |
| rs6495131 | rs55655136 | CSK | SCAMP5 |
| rs6495131 | rs55655136 | CSK | MPI |
| rs6495131 | rs55655136 | CSK | CSK |
| rs6495131 | rs55655136 | CSK | CSK |
| rs6495131 | rs55655136 | CSK | CSK |
| rs6495131 | rs55655136 | CSK | CSK |
| rs6495131 | rs55655136 | CSK | EDC3 |
| rs6495131 | rs55655136 | CSK | EDC3 |
| rs6495131 | rs55655136 | CSK | MIR4513 |
| rs6495131 | rs55655136 | CSK | CPLX3 |
| rs6495131 | rs55655136 | CSK | UBL7-AS1 |
| rs6495131 | rs55655136 | CSK | CTD-3154N5.1 |
| rs6495131 | rs55655136 | CSK | ULK3 |
| rs6495131 | rs55655136 | CSK | FAM219B |
| rs6495131 | rs55655136 | CSK | FAM219B |
| rs6495131 | rs55655136 | CSK | UBL7 |
| rs34323 | rs34330 | CDKN1B | LOH12CR2 |
| rs34323 | rs34330 | CDKN1B | GPR19 |
| rs34323 | rs34330 | CDKN1B | CDKN1B |
| rs34323 | rs34330 | CDKN1B | DUSP16 |
| rs34323 | rs34330 | CDKN1B | DUSP16 |
| rs34323 | rs34330 | CDKN1B | TCONS_00021074 |
| rs34323 | rs34330 | CDKN1B | MANSC1 |
| rs34323 | rs34330 | CDKN1B | LOH12CR1 |
| rs34323 | rs34330 | CDKN1B | TCONS_00020168 |
| rs326614 | rs4388254 | TCF7 | UBE2B |
| rs326614 | rs4388254 | TCF7 | SKP1 |
| rs326614 | rs4388254 | TCF7 | CDKL3 |
| rs11606631 | rs3794060 | DHCR7 | TCONS_00019374 |
| rs11606631 | rs3794060 | DHCR7 | RP11-660L16.2 |
| rs11606631 | rs3794060 | DHCR7 | DHCR7 |
| rs11606631 | rs7118246 | DHCR7 | TCONS_00019374 |
| rs11606631 | rs7118246 | DHCR7 | RP11-660L16.2 |
| rs11606631 | rs7118246 | DHCR7 | DHCR7 |
| rs376442058 | rs1150757 | MHC | NFKBIL1 |
| rs376442058 | rs1150757 | MHC | CLIC1 |
| rs376442058 | rs1150757 | MHC | PRRC2A |
| rs376442058 | rs1150757 | MHC | C6orf47 |
| rs376442058 | rs1150757 | MHC | ATP6V1G2-DDX39B |
| rs376442058 | rs1150757 | MHC | SNORD48 |
| rs376442058 | rs1150757 | MHC | GPANK1 |
| rs376442058 | rs1150757 | MHC | GPANK1 |
| rs376442058 | rs1150757 | MHC | C2 |
| rs376442058 | rs1150757 | MHC | C2 |
| rs376442058 | rs1150757 | MHC | CSNK2B-LY6G5B-1181 |
| rs376442058 | rs1150757 | MHC | CSNK2B-LY6G5B-1181 |
| rs376442058 | rs1150757 | MHC | SNORD84 |
| rs376442058 | rs1150757 | MHC | DDX39B-AS1 |
| rs376442058 | rs1150757 | MHC | ABHD16A |
| rs376442058 | rs1150757 | MHC | ATP6V1G2 |
| rs376442058 | rs1150757 | MHC | C6orf48 |
| rs376442058 | rs1150757 | MHC | DDX39B |
| rs376442058 | rs1150757 | MHC | CSNK2B |
| rs376442058 | rs1150757 | MHC | CSNK2B |
| rs376442058 | rs1150757 | MHC | HSPA1L |
| rs376442058 | rs1150757 | MHC | EHMT2 |
| rs376442058 | rs1150757 | MHC | ZBTB12 |
| rs376442058 | rs1150757 | MHC | LSM2 |
| rs376442058 | rs1150757 | MHC | TNF |
| rs376442058 | rs1150757 | MHC | HSPA1A |
| rs6457791 | rs9462027 | UHRF1BP1 | RP11-140K17.3 |
| rs6457791 | rs9462027 | UHRF1BP1 | C6orf106 |
| rs6457791 | rs9462027 | UHRF1BP1 | SNRPC |
| rs3215823 | rs34889541 | PTPRC | MIR181A1HG |
| rs3215823 | rs34889541 | PTPRC | TCONS_00000111 |
| rs3215823 | rs34889541 | PTPRC | RP11-553K8.2 |
| rs3215823 | rs34889541 | PTPRC | NEK7 |
| 12:12878679 | rs34330 | CDKN1B | LOH12CR2 |
| 12:12878679 | rs34330 | CDKN1B | GPR19 |
| 12:12878679 | rs34330 | CDKN1B | CDKN1B |
| 12:12878679 | rs34330 | CDKN1B | DUSP16 |
| 12:12878679 | rs34330 | CDKN1B | DUSP16 |
| 12:12878679 | rs34330 | CDKN1B | TCONS_00021074 |
| 12:12878679 | rs34330 | CDKN1B | MANSC1 |
| 12:12878679 | rs34330 | CDKN1B | LOH12CR1 |
| 12:12878679 | rs34330 | CDKN1B | TCONS_00020168 |
| rs12789751 | rs3794060 | DHCR7 | TCONS_00019374 |
| rs12789751 | rs3794060 | DHCR7 | RP11-660L16.2 |
| rs12789751 | rs3794060 | DHCR7 | DHCR7 |
| rs12789751 | rs7118246 | DHCR7 | TCONS_00019374 |
| rs12789751 | rs7118246 | DHCR7 | RP11-660L16.2 |
| rs12789751 | rs7118246 | DHCR7 | DHCR7 |
| rs1645934 | rs1170426 | ZFP90 | CHTF8 |
| rs1645934 | rs1170426 | ZFP90 | CIRH1A |
| rs3807307 | rs3757387 | IRF5 | TSPAN33 |
| rs3807307 | rs3757387 | IRF5 | CCDC136 |
| rs34613499 | rs10048743 | IKZF2 | RP11-105N14.1 |
| rs34613499 | rs10048743 | IKZF2 | IKZF2 |
| rs36041042 | rs12938617 | IKZF3 | IKZF3 |
| rs36041042 | rs12938617 | IKZF3 | IKZF3 |
| rs36041042 | rs12938617 | IKZF3 | ZPBP2 |
| rs36041042 | rs2941509 | IKZF3 | IKZF3 |
| rs36041042 | rs2941509 | IKZF3 | IKZF3 |
| rs36041042 | rs2941509 | IKZF3 | ZPBP2 |
| rs527619 | rs4639966 | Intergenic | DPAGT1 |
| rs527619 | rs4639966 | Intergenic | BCL9L |
| rs527619 | rs4639966 | Intergenic | C2CD2L |
| rs527619 | rs4639966 | Intergenic | DDX6 |
| rs527619 | rs4639966 | Intergenic | DDX6 |
| rs527619 | rs4639966 | Intergenic | TCONS_00020001 |
| rs527619 | rs4639966 | Intergenic | CXCR5 |
| rs527619 | rs4639966 | Intergenic | TCONS_00019492 |
| rs527619 | rs4639966 | Intergenic | MIR4492 |
| rs111352543 | rs12917716 | CIITA | RP11-485G7.5 |
| rs111352543 | rs12917716 | CIITA | RMI2 |
| rs111352543 | rs9652601 | CIITA | RP11-485G7.5 |
| rs111352543 | rs9652601 | CIITA | RMI2 |
| rs173424 | rs244687 | TCF7 | SAR1B |
| rs173424 | rs244687 | TCF7 | SEC24A |
| rs173424 | rs244687 | TCF7 | CTD-2410N18.5 |
| rs173424 | rs244687 | TCF7 | CDKL3 |
| rs173424 | rs244687 | TCF7 | CTD-2410N18.3 |
| rs173424 | rs244687 | TCF7 | VDAC1 |
| rs173424 | rs244687 | TCF7 | PPP2CA |
| rs173424 | rs244687 | TCF7 | UBE2B |
| rs173424 | rs244687 | TCF7 | CTB-113I20.2 |
| rs173424 | rs244687 | TCF7 | PHF15 |
| rs173424 | rs244687 | TCF7 | PHF15 |
| rs173424 | rs244687 | TCF7 | PHF15 |
| rs173424 | rs244687 | TCF7 | MIR3661 |
| rs173424 | rs244687 | TCF7 | TCF7 |
| rs173424 | rs244687 | TCF7 | TCF7 |
| rs75142770 | rs1061502 | IRF7 | TCONS_00019162 |
| rs75142770 | rs1061502 | IRF7 | PHRF1 |
| rs11552449 | rs11102701 | PTPN22 | RP5-1073O3.2 |
| rs11552449 | rs11102701 | PTPN22 | RP5-1073O3.7 |
| rs11552449 | rs11102701 | PTPN22 | HIPK1 |
| rs11552449 | rs11102701 | PTPN22 | CSDE1 |
| rs11552449 | rs11102701 | PTPN22 | PTPN22 |
| rs11552449 | rs11102701 | PTPN22 | AP4B1 |
| rs11552449 | rs11102701 | PTPN22 | DCLRE1B |
| rs11552449 | rs11102701 | PTPN22 | RSBN1 |
| rs11552449 | rs11102701 | PTPN22 | PHTF1 |
| rs2002064 | rs3794060 | DHCR7 | TCONS_00019374 |
| rs2002064 | rs3794060 | DHCR7 | RP11-660L16.2 |
| rs2002064 | rs3794060 | DHCR7 | DHCR7 |
| rs2002064 | rs7118246 | DHCR7 | TCONS_00019374 |
| rs2002064 | rs7118246 | DHCR7 | RP11-660L16.2 |
| rs2002064 | rs7118246 | DHCR7 | DHCR7 |
| rs12791871 | rs3794060 | DHCR7 | TCONS_00019374 |
| rs12791871 | rs3794060 | DHCR7 | RP11-660L16.2 |
| rs12791871 | rs3794060 | DHCR7 | DHCR7 |
| rs12791871 | rs7118246 | DHCR7 | TCONS_00019374 |
| rs12791871 | rs7118246 | DHCR7 | RP11-660L16.2 |
| rs12791871 | rs7118246 | DHCR7 | DHCR7 |
| rs12709364 | rs12938617 | IKZF3 | CASC3 |
| rs12709364 | rs12938617 | IKZF3 | IKZF3 |
| rs12709364 | rs12938617 | IKZF3 | IKZF3 |
| rs12709364 | rs12938617 | IKZF3 | ZPBP2 |
| rs12709364 | rs2941509 | IKZF3 | CASC3 |
| rs12709364 | rs2941509 | IKZF3 | IKZF3 |
| rs12709364 | rs2941509 | IKZF3 | IKZF3 |
| rs12709364 | rs2941509 | IKZF3 | ZPBP2 |
| rs10995021 | rs10761602 | ARID5B | RTKN2 |
| rs10995021 | rs10761602 | ARID5B | ARID5B |
| rs10995021 | rs10761602 | ARID5B | ARID5B |
| rs10995021 | rs10761602 | ARID5B | ARID5B |
| rs10995021 | rs4948496 | ARID5B | RTKN2 |
| rs10995021 | rs4948496 | ARID5B | ARID5B |
| rs10995021 | rs4948496 | ARID5B | ARID5B |
| rs10995021 | rs4948496 | ARID5B | ARID5B |
| rs77599345 | rs9652601 | CIITA | RP11-490O6.2 |
| rs77599345 | rs9652601 | CIITA | RP11-485G7.5 |
| rs77599345 | rs9652601 | CIITA | SOCS1 |
| rs77599345 | rs9652601 | CIITA | TXNDC11 |
| rs77599345 | rs9652601 | CIITA | RMI2 |
| rs72787722 | rs17321999 | LBH | TCONS_00003622 |
| rs72787722 | rs17321999 | LBH | LCLAT1 |
| rs72787722 | rs17321999 | LBH | TCONS_00004194 |
| rs72787722 | rs17321999 | LBH | TCONS_00004193 |
| rs72787722 | rs17321999 | LBH | TCONS_00005128 |
| rs34135674 | rs12938617 | IKZF3 | IKZF3 |
| rs34135674 | rs12938617 | IKZF3 | IKZF3 |
| rs34135674 | rs12938617 | IKZF3 | ZPBP2 |
| rs34135674 | rs2941509 | IKZF3 | IKZF3 |
| rs34135674 | rs2941509 | IKZF3 | IKZF3 |
| rs34135674 | rs2941509 | IKZF3 | ZPBP2 |
| rs3117583 | rs1150757 | MHC | NFKBIL1 |
| rs3117583 | rs1150757 | MHC | CLIC1 |
| rs3117583 | rs1150757 | MHC | PRRC2A |
| rs3117583 | rs1150757 | MHC | C6orf47 |
| rs3117583 | rs1150757 | MHC | ATP6V1G2-DDX39B |
| rs3117583 | rs1150757 | MHC | SNORD48 |
| rs3117583 | rs1150757 | MHC | GPANK1 |
| rs3117583 | rs1150757 | MHC | GPANK1 |
| rs3117583 | rs1150757 | MHC | C2 |
| rs3117583 | rs1150757 | MHC | C2 |
| rs3117583 | rs1150757 | MHC | CSNK2B-LY6G5B-1181 |
| rs3117583 | rs1150757 | MHC | CSNK2B-LY6G5B-1181 |
| rs3117583 | rs1150757 | MHC | SNORD84 |
| rs3117583 | rs1150757 | MHC | DDX39B-AS1 |
| rs3117583 | rs1150757 | MHC | ABHD16A |
| rs3117583 | rs1150757 | MHC | ATP6V1G2 |
| rs3117583 | rs1150757 | MHC | C6orf48 |
| rs3117583 | rs1150757 | MHC | DDX39B |
| rs3117583 | rs1150757 | MHC | CSNK2B |
| rs3117583 | rs1150757 | MHC | CSNK2B |
| rs3117583 | rs1150757 | MHC | HSPA1L |
| rs3117583 | rs1150757 | MHC | EHMT2 |
| rs3117583 | rs1150757 | MHC | ZBTB12 |
| rs3117583 | rs1150757 | MHC | LSM2 |
| rs3117583 | rs1150757 | MHC | TNF |
| rs3117583 | rs1150757 | MHC | HSPA1A |
| rs34793529 | rs12418883 | IRF7 | DEAF1 |
| rs34793529 | rs12418883 | IRF7 | DEAF1 |
| rs34793529 | rs12418883 | IRF7 | HRAS |
| rs34793529 | rs12418883 | IRF7 | HRAS |
| rs34793529 | rs12418883 | IRF7 | AP006621.9 |
| rs34793529 | rs12418883 | IRF7 | AP006621.9 |
| rs34793529 | rs12418883 | IRF7 | RNH1 |
| rs34793529 | rs12418883 | IRF7 | EPS8L2 |
| rs34793529 | rs12418883 | IRF7 | EPS8L2 |
| rs34793529 | rs12418883 | IRF7 | EPS8L2 |
| rs34793529 | rs12418883 | IRF7 | PSMD13 |
| rs34793529 | rs12418883 | IRF7 | LRRC56 |
| rs34793529 | rs12418883 | IRF7 | LRRC56 |
| rs34793529 | rs12418883 | IRF7 | PIDD |
| rs34793529 | rs12418883 | IRF7 | TMEM80 |
| rs34793529 | rs12418883 | IRF7 | CEND1 |
| rs34793529 | rs12418883 | IRF7 | RASSF7 |
| rs34793529 | rs12418883 | IRF7 | SIRT3 |
| rs34793529 | rs12418883 | IRF7 | C11orf35 |
| rs34793529 | rs12418883 | IRF7 | IRF7 |
| rs34793529 | rs1061502 | IRF7 | DEAF1 |
| rs34793529 | rs1061502 | IRF7 | DEAF1 |
| rs34793529 | rs1061502 | IRF7 | HRAS |
| rs34793529 | rs1061502 | IRF7 | HRAS |
| rs34793529 | rs1061502 | IRF7 | AP006621.9 |
| rs34793529 | rs1061502 | IRF7 | AP006621.9 |
| rs34793529 | rs1061502 | IRF7 | RNH1 |
| rs34793529 | rs1061502 | IRF7 | EPS8L2 |
| rs34793529 | rs1061502 | IRF7 | EPS8L2 |
| rs34793529 | rs1061502 | IRF7 | EPS8L2 |
| rs34793529 | rs1061502 | IRF7 | PSMD13 |
| rs34793529 | rs1061502 | IRF7 | LRRC56 |
| rs34793529 | rs1061502 | IRF7 | LRRC56 |
| rs34793529 | rs1061502 | IRF7 | PIDD |
| rs34793529 | rs1061502 | IRF7 | TMEM80 |
| rs34793529 | rs1061502 | IRF7 | CEND1 |
| rs34793529 | rs1061502 | IRF7 | RASSF7 |
| rs34793529 | rs1061502 | IRF7 | SIRT3 |
| rs34793529 | rs1061502 | IRF7 | C11orf35 |
| rs34793529 | rs1061502 | IRF7 | IRF7 |
| rs7902719 | rs10761602 | ARID5B | RTKN2 |
| rs7902719 | rs10761602 | ARID5B | ARID5B |
| rs7902719 | rs4948496 | ARID5B | RTKN2 |
| rs7902719 | rs4948496 | ARID5B | ARID5B |
| rs4843866 | rs13332649 | IRF8 | IRF8 |
| rs4843866 | rs13332649 | IRF8 | TCONS_00024489 |
| rs36228499 | rs34330 | CDKN1B | LOH12CR2 |
| rs36228499 | rs34330 | CDKN1B | GPR19 |
| rs36228499 | rs34330 | CDKN1B | TCONS_00021074 |
| rs36228499 | rs34330 | CDKN1B | DUSP16 |
| rs36228499 | rs34330 | CDKN1B | DUSP16 |
| rs36228499 | rs34330 | CDKN1B | APOLD1 |
| rs36228499 | rs34330 | CDKN1B | APOLD1 |
| rs36228499 | rs34330 | CDKN1B | RP11-180M15.4 |
| rs36228499 | rs34330 | CDKN1B | RP11-180M15.4 |
| rs36228499 | rs34330 | CDKN1B | MANSC1 |
| rs36228499 | rs34330 | CDKN1B | LOH12CR1 |
| rs36228499 | rs34330 | CDKN1B | TCONS_00020168 |
| rs73923215 | rs2304256 | TYK2 | CTD-2369P2.4 |
| rs73923215 | rs2304256 | TYK2 | TCONS_I2_00012303 |
| rs73923215 | rs2304256 | TYK2 | ICAM1 |
| rs73923215 | rs2304256 | TYK2 | ICAM1 |
| rs73923215 | rs2304256 | TYK2 | PDE4A |
| rs73923215 | rs2304256 | TYK2 | PDE4A |
| rs73923215 | rs11085725 | TYK2 | CTD-2369P2.4 |
| rs73923215 | rs11085725 | TYK2 | TCONS_I2_00012303 |
| rs73923215 | rs11085725 | TYK2 | ICAM1 |
| rs73923215 | rs11085725 | TYK2 | ICAM1 |
| rs73923215 | rs11085725 | TYK2 | PDE4A |
| rs73923215 | rs11085725 | TYK2 | PDE4A |
| rs33934794 | rs6889239 | TNIP1 | ANXA6 |
| rs33934794 | rs6889239 | TNIP1 | TNIP1 |
| rs33934794 | rs6889239 | TNIP1 | DCTN4 |
| rs33934794 | rs6889239 | TNIP1 | ATOX1 |
| rs33934794 | rs6889239 | TNIP1 | G3BP1 |
| rs33934794 | rs6889239 | TNIP1 | GM2A |
| rs33934794 | rs6889239 | TNIP1 | CCDC69 |
| rs33934794 | rs10036748 | TNIP1 | ANXA6 |
| rs33934794 | rs10036748 | TNIP1 | TNIP1 |
| rs33934794 | rs10036748 | TNIP1 | DCTN4 |
| rs33934794 | rs10036748 | TNIP1 | ATOX1 |
| rs33934794 | rs10036748 | TNIP1 | G3BP1 |
| rs33934794 | rs10036748 | TNIP1 | GM2A |
| rs33934794 | rs10036748 | TNIP1 | CCDC69 |
| rs4781030 | rs9652601 | CIITA | RP11-485G7.5 |
| rs4781030 | rs9652601 | CIITA | RMI2 |
| rs71297581 | rs2304256 | TYK2 | FDX1L |
| rs71297581 | rs2304256 | TYK2 | CTD-2369P2.4 |
| rs71297581 | rs2304256 | TYK2 | CTD-2369P2.10 |
| rs71297581 | rs2304256 | TYK2 | RAVER1 |
| rs71297581 | rs2304256 | TYK2 | ICAM1 |
| rs71297581 | rs2304256 | TYK2 | ICAM1 |
| rs71297581 | rs2304256 | TYK2 | ICAM1 |
| rs71297581 | rs11085725 | TYK2 | FDX1L |
| rs71297581 | rs11085725 | TYK2 | CTD-2369P2.4 |
| rs71297581 | rs11085725 | TYK2 | CTD-2369P2.10 |
| rs71297581 | rs11085725 | TYK2 | RAVER1 |
| rs71297581 | rs11085725 | TYK2 | ICAM1 |
| rs71297581 | rs11085725 | TYK2 | ICAM1 |
| rs71297581 | rs11085725 | TYK2 | ICAM1 |
| rs3131383 | rs1150757 | MHC | VWA7 |
| rs3131383 | rs1150757 | MHC | DDAH2 |
| rs3131383 | rs1150757 | MHC | CLIC1 |
| rs3131383 | rs1150757 | MHC | CLIC1 |
| rs3131383 | rs1150757 | MHC | NFKBIL1 |
| rs3131383 | rs1150757 | MHC | VARS |
| rs3131383 | rs1150757 | MHC | TCONS_000H779 |
| rs3131383 | rs1150757 | MHC | PRRC2A |
| rs3131383 | rs1150757 | MHC | C6orf47 |
| rs3131383 | rs1150757 | MHC | APOM |
| rs3131383 | rs1150757 | MHC | ATP6V1G2-DDX39B |
| rs3131383 | rs1150757 | MHC | SNORD48 |
| rs3131383 | rs1150757 | MHC | GPANK1 |
| rs3131383 | rs1150757 | MHC | GPANK1 |
| rs3131383 | rs1150757 | MHC | C2 |
| rs3131383 | rs1150757 | MHC | CSNK2B-LY6G5B-1181 |
| rs3131383 | rs1150757 | MHC | CSNK2B-LY6G5B-1181 |
| rs3131383 | rs1150757 | MHC | NEU1 |
| rs3131383 | rs1150757 | MHC | NELFE |
| rs3131383 | rs1150757 | MHC | SNORD84 |
| rs3131383 | rs1150757 | MHC | DDX39B-AS1 |
| rs3131383 | rs1150757 | MHC | ABHD16A |
| rs3131383 | rs1150757 | MHC | LY6G5C |
| rs3131383 | rs1150757 | MHC | ATP6V1G2 |
| rs3131383 | rs1150757 | MHC | SKIV2L |
| rs3131383 | rs1150757 | MHC | BAG6 |
| rs3131383 | rs1150757 | MHC | C6orf48 |
| rs3131383 | rs1150757 | MHC | DDX39B |
| rs3131383 | rs1150757 | MHC | CSNK2B |
| rs3131383 | rs1150757 | MHC | CSNK2B |
| rs3131383 | rs1150757 | MHC | HSPA1L |
| rs3131383 | rs1150757 | MHC | EHMT2 |
| rs3131383 | rs1150757 | MHC | LSM2 |
| rs3131383 | rs1150757 | MHC | HSPA1A |
| rs71368520 | rs2286672 | PLD2 | MINK1 |
| rs12128057 | rs34889541 | PTPRC | PTPRC |
| rs9896218 | rs12938617 | IKZF3 | CDK12 |
| rs9896218 | rs12938617 | IKZF3 | GRB7 |
| rs9896218 | rs12938617 | IKZF3 | STARD3 |
| rs9896218 | rs12938617 | IKZF3 | TCAP |
| rs9896218 | rs12938617 | IKZF3 | PGAP3 |
| rs9896218 | rs12938617 | IKZF3 | PNMT |
| rs9896218 | rs12938617 | IKZF3 | ERBB2 |
| rs9896218 | rs2941509 | IKZF3 | CDK12 |
| rs9896218 | rs2941509 | IKZF3 | GRB7 |
| rs9896218 | rs2941509 | IKZF3 | STARD3 |
| rs9896218 | rs2941509 | IKZF3 | TCAP |
| rs9896218 | rs2941509 | IKZF3 | PGAP3 |
| rs9896218 | rs2941509 | IKZF3 | PNMT |
| rs9896218 | rs2941509 | IKZF3 | ERBB2 |
| rs6934662 | rs9462027 | UHRF1BP1 | RP11-140K17.3 |
| rs6934662 | rs9462027 | UHRF1BP1 | C6orf106 |
| rs6934662 | rs9462027 | UHRF1BP1 | SNRPC |
| rs6930933 | rs1150757 | MHC | HLA-DRA |
| rs1004379 | rs34548580 | PLD2 | SLC25A11 |
| rs1004379 | rs34548580 | PLD2 | RNF167 |
| rs1004379 | rs34548580 | PLD2 | CYB5D2 |
| rs1004379 | rs34548580 | PLD2 | ZZEF1 |
| rs112743130 | rs12938617 | IKZF3 | IKZF3 |
| rs112743130 | rs12938617 | IKZF3 | IKZF3 |
| rs112743130 | rs2941509 | IKZF3 | IKZF3 |
| rs112743130 | rs2941509 | IKZF3 | IKZF3 |
| rs62141147 | rs7579944 | LBH | TCONS_00003622 |
| rs62141147 | rs7579944 | LBH | LCLAT1 |
| rs62141147 | rs7579944 | LBH | TCONS_00004194 |
| rs62141147 | rs7579944 | LBH | TCONS_00004193 |
| rs62141147 | rs7579944 | LBH | TCONS_00005128 |
| rs12357548 | rs10761602 | ARID5B | RTKN2 |
| rs12357548 | rs10761602 | ARID5B | ARID5B |
| rs12357548 | rs10761602 | ARID5B | ARID5B |
| rs12357548 | rs4948496 | ARID5B | RTKN2 |
| rs12357548 | rs4948496 | ARID5B | ARID5B |
| rs12357548 | rs4948496 | ARID5B | ARID5B |
| rs2297550 | rs2297550 | IKBKE | DYRK3 |
| rs2297550 | rs2297550 | IKBKE | RASSF5 |
| rs2297550 | rs2297550 | IKBKE | RASSF5 |
| rs2297550 | rs2297550 | IKBKE | RASSF5 |
| rs2297550 | rs2297550 | IKBKE | RASSF5 |
| rs2297550 | rs2297550 | IKBKE | EIF2D |
| rs2297550 | rs2297550 | IKBKE | MAPKAPK2 |
| rs2297550 | rs2297550 | IKBKE | RP11-343H5.6 |
| rs75752784 | rs4388254 | TCF7 | PHF15 |
| rs75752784 | rs4388254 | TCF7 | PHF15 |
| rs75752784 | rs4388254 | TCF7 | PHF15 |
| rs75752784 | rs4388254 | TCF7 | TCF7 |
| rs75752784 | rs4388254 | TCF7 | TCF7 |
| rs75752784 | rs4388254 | TCF7 | AC005355.1 |
| rs35593987 | rs11889341 | STAT4 | STAT4 |
| rs35593987 | rs4274624 | STAT4 | STAT4 |
| rs16843474 | rs34889541 | PTPRC | PTPRC |
| rs11055024 | rs34330 | CDKN1B | DUSP16 |
| rs11055024 | rs34330 | CDKN1B | DUSP16 |
| rs11055024 | rs10845601 | CDKN1B | DUSP16 |
| rs11055024 | rs10845601 | CDKN1B | DUSP16 |
| rs1728773 | rs1170426 | ZFP90 | CHTF8 |
| rs1728773 | rs1170426 | ZFP90 | CIRH1A |
| rs3130631 | rs1150757 | MHC | SNORD84 |
| rs3130631 | rs1150757 | MHC | DDX39B-AS1 |
| rs3130631 | rs1150757 | MHC | DDX39B |
| rs3130631 | rs1150757 | MHC | ATP6V1G2-DDX39B |
| rs3130631 | rs1150757 | MHC | ATP6V1G2-DDX39B |
| rs3130631 | rs1150757 | MHC | NFKBIL1 |
| rs3130631 | rs1150757 | MHC | NFKBIL1 |
| rs3130631 | rs1150757 | MHC | ATP6V1G2 |
| rs3130631 | rs1150757 | MHC | ATP6V1G2 |
| rs4660117 | rs9782955 | LYST | B3GALNT2 |
| rs4660117 | rs9782955 | LYST | LYST |
| rs9915509 | rs12938617 | IKZF3 | IKZF3 |
| rs9915509 | rs2941509 | IKZF3 | IKZF3 |
| rs3805431 | rs6889239 | TNIP1 | GM2A |
| rs3805431 | rs6889239 | TNIP1 | TNIP1 |
| rs3805431 | rs6889239 | TNIP1 | TNIP1 |
| rs3805431 | rs6889239 | TNIP1 | DCTN4 |
| rs3805431 | rs10036748 | TNIP1 | GM2A |
| rs3805431 | rs10036748 | TNIP1 | TNIP1 |
| rs3805431 | rs10036748 | TNIP1 | TNIP1 |
| rs3805431 | rs10036748 | TNIP1 | DCTN4 |
| rs3087876 | rs12917716 | CIITA | GSPT1 |
| rs3087876 | rs12917716 | CIITA | AC007216.1 |
| rs3087876 | rs12917716 | CIITA | RP11-485G7.5 |
| rs3087876 | rs12917716 | CIITA | RMI2 |
| rs3087876 | rs9652601 | CIITA | GSPT1 |
| rs3087876 | rs9652601 | CIITA | AC007216.1 |
| rs3087876 | rs9652601 | CIITA | RP11-485G7.5 |
| rs3087876 | rs9652601 | CIITA | RMI2 |
| rs7743724 | rs9462027 | UHRF1BP1 | ANKS1A |
| rs7743724 | rs9462027 | UHRF1BP1 | ANKS1A |
| rs7743724 | rs9462027 | UHRF1BP1 | TAF11 |
| rs7743724 | rs9462027 | UHRF1BP1 | TAF11 |
| rs7743724 | rs9462027 | UHRF1BP1 | UHRF1BP1 |
| rs9901483 | rs12938617 | IKZF3 | IKZF3 |
| rs9901483 | rs12938617 | IKZF3 | IKZF3 |
| rs9901483 | rs12938617 | IKZF3 | ZPBP2 |
| rs9901483 | rs2941509 | IKZF3 | IKZF3 |
| rs9901483 | rs2941509 | IKZF3 | IKZF3 |
| rs9901483 | rs2941509 | IKZF3 | ZPBP2 |
| rs10499197 | rs77000060 | TNFAIP3 | TNFAIP3 |
| rs10499197 | rs77000060 | TNFAIP3 | RP11-356I2.4 |
| rs10499197 | rs2230926 | TNFAIP3 | TNFAIP3 |
| rs10499197 | rs2230926 | TNFAIP3 | RP11-356I2.4 |
| rs2509948 | rs494003 | RNASEH2C | LTBP3 |
| rs2509948 | rs494003 | RNASEH2C | DRAP1 |
| rs2509948 | rs494003 | RNASEH2C | PCNXL3 |
| rs2509948 | rs494003 | RNASEH2C | MAP3K11 |
| rs2509948 | rs494003 | RNASEH2C | EIF1AD |
| rs2509948 | rs494003 | RNASEH2C | MUS81 |
| rs2509948 | rs494003 | RNASEH2C | SIPA1 |
| rs2509948 | rs494003 | RNASEH2C | TCONS_I2_00005065 |
| rs2509948 | rs494003 | RNASEH2C | SNX32 |
| rs2509948 | rs494003 | RNASEH2C | SCYL1 |
| rs2509948 | rs494003 | RNASEH2C | C11orf68 |
| rs2509948 | rs494003 | RNASEH2C | CFL1 |
| rs2509948 | rs494003 | RNASEH2C | CFL1 |
| rs2509948 | rs494003 | RNASEH2C | FAM89B |
| rs2509948 | rs494003 | RNASEH2C | BANF1 |
| rs2509948 | rs494003 | RNASEH2C | EHBP1L1 |
| rs2509948 | rs494003 | RNASEH2C | EHBP1L1 |
| rs2509948 | rs494003 | RNASEH2C | TCONS_I2_00005064 |
| rs12919083 | rs12917716 | CIITA | RP11-485G7.5 |
| rs12919083 | rs12917716 | CIITA | RMI2 |
| rs12919083 | rs9652601 | CIITA | RP11-485G7.5 |
| rs12919083 | rs9652601 | CIITA | RMI2 |
| rs8052690 | rs13332649 | IRF8 | IRF8 |
| rs8052690 | rs13332649 | IRF8 | TCONS_00024489 |
| rs4764683 | rs4622329 | DRAM1 | IGF1 |
| rs66561220 | rs34330 | CDKN1B | LOH12CR2 |
| rs66561220 | rs34330 | CDKN1B | GPR19 |
| rs66561220 | rs34330 | CDKN1B | CDKN1B |
| rs66561220 | rs34330 | CDKN1B | DUSP16 |
| rs66561220 | rs34330 | CDKN1B | DUSP16 |
| rs66561220 | rs34330 | CDKN1B | TCONS_00021074 |
| rs66561220 | rs34330 | CDKN1B | MANSC1 |
| rs66561220 | rs34330 | CDKN1B | LOH12CR1 |
| rs66561220 | rs34330 | CDKN1B | TCONS_00020168 |
| rs9268532 | rs1150757 | MHC | HLA-DRA |
| rs16843471 | rs34889541 | PTPRC | PTPRC |
| rs34324 | rs34330 | CDKN1B | LOH12CR2 |
| rs34324 | rs34330 | CDKN1B | GPR19 |
| rs34324 | rs34330 | CDKN1B | CDKN1B |
| rs34324 | rs34330 | CDKN1B | DUSP16 |
| rs34324 | rs34330 | CDKN1B | DUSP16 |
| rs34324 | rs34330 | CDKN1B | TCONS_00021074 |
| rs34324 | rs34330 | CDKN1B | MANSC1 |
| rs34324 | rs34330 | CDKN1B | LOH12CR1 |
| rs34324 | rs34330 | CDKN1B | TCONS_00020168 |
| rs2002063 | rs3794060 | DHCR7 | TCONS_00019374 |
| rs2002063 | rs3794060 | DHCR7 | RP11-660L16.2 |
| rs2002063 | rs3794060 | DHCR7 | DHCR7 |
| rs2002063 | rs7118246 | DHCR7 | TCONS_00019374 |
| rs2002063 | rs7118246 | DHCR7 | RP11-660L16.2 |
| rs2002063 | rs7118246 | DHCR7 | DHCR7 |
| rs11130642 | rs9311676 | ABHD6 | RP11-359I18.5 |
| rs11130642 | rs9311676 | ABHD6 | KCTD6 |
| rs11130642 | rs9311676 | ABHD6 | PXK |
| rs11130642 | rs9311676 | ABHD6 | RPP14 |
| rs3169572 | rs12938617 | IKZF3 | LRRC3C |
| rs3169572 | rs12938617 | IKZF3 | ZPBP2 |
| rs3169572 | rs2941509 | IKZF3 | LRRC3C |
| rs3169572 | rs2941509 | IKZF3 | ZPBP2 |
| rs6498136 | rs12917716 | CIITA | GSPT1 |
| rs6498136 | rs12917716 | CIITA | AC007216.1 |
| rs6498136 | rs12917716 | CIITA | RP11-485G7.5 |
| rs6498136 | rs12917716 | CIITA | RMI2 |
| rs6498136 | rs9652601 | CIITA | GSPT1 |
| rs6498136 | rs9652601 | CIITA | AC007216.1 |
| rs6498136 | rs9652601 | CIITA | RP11-485G7.5 |
| rs6498136 | rs9652601 | CIITA | RMI2 |
| rs28403951 | rs12938617 | IKZF3 | IKZF3 |
| rs28403951 | rs12938617 | IKZF3 | IKZF3 |
| rs28403951 | rs12938617 | IKZF3 | ZPBP2 |
| rs28403951 | rs2941509 | IKZF3 | IKZF3 |
| rs28403951 | rs2941509 | IKZF3 | IKZF3 |
| rs28403951 | rs2941509 | IKZF3 | ZPBP2 |
| rs62141110 | rs268124 | SPRED2 | ACTR2 |
| rs62141110 | rs268124 | SPRED2 | SPRED2 |
| rs62141110 | rs268124 | SPRED2 | SPRED2 |
| rs62141110 | rs268124 | SPRED2 | SPRED2 |
| rs1478890 | rs4840568 | BLK | FDFT1 |
| rs1478890 | rs2736332 | BLK | FDFT1 |
| rs7205916 | rs12917716 | CIITA | RP11-485G7.5 |
| rs7205916 | rs12917716 | CIITA | RMI2 |
| rs703711 | rs9804869 | DRAM1 | ACTR6 |
| rs703711 | rs9804869 | DRAM1 | RP11-175P13.3 |
| rs1170445 | rs1170426 | ZFP90 | CHTF8 |
| rs1170445 | rs1170426 | ZFP90 | CIRH1A |
| rs35938199 | rs12938617 | IKZF3 | IKZF3 |
| rs35938199 | rs12938617 | IKZF3 | IKZF3 |
| rs35938199 | rs2941509 | IKZF3 | IKZF3 |
| rs35938199 | rs2941509 | IKZF3 | IKZF3 |
| rs11600860 | rs3794060 | DHCR7 | TCONS_00019374 |
| rs11600860 | rs3794060 | DHCR7 | RP11-660L16.2 |
| rs11600860 | rs3794060 | DHCR7 | DHCR7 |
| rs11600860 | rs7118246 | DHCR7 | TCONS_00019374 |
| rs11600860 | rs7118246 | DHCR7 | RP11-660L16.2 |
| rs11600860 | rs7118246 | DHCR7 | DHCR7 |
| rs2086643 | rs10048743 | IKZF2 | RP11-105N14.1 |
| rs2086643 | rs10048743 | IKZF2 | IKZF2 |
| rs72736070 | rs34889541 | PTPRC | PTPRC |
| rs7769820 | rs9462027 | UHRF1BP1 | RP11-140K17.3 |
| rs7769820 | rs9462027 | UHRF1BP1 | C6orf106 |
| rs7769820 | rs9462027 | UHRF1BP1 | SNRPC |
| rs201152777 | rs12938617 | IKZF3 | IKZF3 |
| rs201152777 | rs2941509 | IKZF3 | IKZF3 |
| rs12937330 | rs12938617 | IKZF3 | IKZF3 |
| rs12937330 | rs2941509 | IKZF3 | IKZF3 |
| rs1645975 | rs1170426 | ZFP90 | CHTF8 |
| rs1645975 | rs1170426 | ZFP90 | CIRH1A |
| rs13215181 | rs9462027 | UHRF1BP1 | RP11-140K17.3 |
| rs13215181 | rs9462027 | UHRF1BP1 | C6orf106 |
| rs13215181 | rs9462027 | UHRF1BP1 | SNRPC |
| rs9268531 | rs1150757 | MHC | HLA-DRA |
| rs6881720 | rs4388254 | TCF7 | SEC24A |
| rs6881720 | rs4388254 | TCF7 | SAR1B |
| rs6881720 | rs4388254 | TCF7 | CTD-2410N18.5 |
| rs6881720 | rs4388254 | TCF7 | AC005355.1 |
| rs6881720 | rs4388254 | TCF7 | CDKL3 |
| rs6881720 | rs4388254 | TCF7 | CDKL3 |
| rs6881720 | rs4388254 | TCF7 | CTD-2410N18.3 |
| rs6881720 | rs4388254 | TCF7 | PPP2CA |
| rs6881720 | rs4388254 | TCF7 | CDKN2AIPNL |
| rs6881720 | rs4388254 | TCF7 | UBE2B |
| rs6881720 | rs4388254 | TCF7 | MIR3661 |
| rs10995020 | rs10761602 | ARID5B | RTKN2 |
| rs10995020 | rs10761602 | ARID5B | ARID5B |
| rs10995020 | rs10761602 | ARID5B | ARID5B |
| rs10995020 | rs10761602 | ARID5B | ARID5B |
| rs10995020 | rs4948496 | ARID5B | RTKN2 |
| rs10995020 | rs4948496 | ARID5B | ARID5B |
| rs10995020 | rs4948496 | ARID5B | ARID5B |
| rs10995020 | rs4948496 | ARID5B | ARID5B |
| rs7683892 | rs6856202 | BANK1 | BANK1 |
| rs7683892 | rs11280314 | BANK1 | BANK1 |
| rs79044630 | rs6762714 | LPP-TPRG1 | RP11-132N15.3 |
| rs79044630 | rs6762714 | LPP-TPRG1 | BCL6 |
| rs79044630 | rs6762714 | LPP-TPRG1 | BCL6 |
| rs8046423 | rs9652601 | CIITA | RP11-490O6.2 |
| rs8046423 | rs9652601 | CIITA | RP11-485G7.5 |
| rs8046423 | rs9652601 | CIITA | SOCS1 |
| rs8046423 | rs9652601 | CIITA | TXNDC11 |
| rs8046423 | rs9652601 | CIITA | RMI2 |
| rs3830584 | rs6762714 | LPP-TPRG1 | BCL6 |
| rs3830584 | rs6762714 | LPP-TPRG1 | BCL6 |
| rs59003594 | rs34889541 | PTPRC | RP11-553K8.2 |
| rs59003594 | rs34889541 | PTPRC | PTPRC |
| rs558702 | rs1150757 | MHC | NFKBIL1 |
| rs558702 | rs1150757 | MHC | APOM |
| rs558702 | rs1150757 | MHC | ATP6V1G2-DDX39B |
| rs558702 | rs1150757 | MHC | GPANK1 |
| rs558702 | rs1150757 | MHC | GPANK1 |
| rs558702 | rs1150757 | MHC | NELFE |
| rs558702 | rs1150757 | MHC | CSNK2B-LY6G5B-1181 |
| rs558702 | rs1150757 | MHC | CSNK2B-LY6G5B-1181 |
| rs558702 | rs1150757 | MHC | SNORD84 |
| rs558702 | rs1150757 | MHC | DDX39B-AS1 |
| rs558702 | rs1150757 | MHC | ABHD16A |
| rs558702 | rs1150757 | MHC | LY6G5C |
| rs558702 | rs1150757 | MHC | ATP6V1G2 |
| rs558702 | rs1150757 | MHC | SKIV2L |
| rs558702 | rs1150757 | MHC | BAG6 |
| rs558702 | rs1150757 | MHC | DDX39B |
| rs558702 | rs1150757 | MHC | CSNK2B |
| rs558702 | rs1150757 | MHC | CSNK2B |
| rs3101018 | rs1150757 | MHC | DXO |
| rs3101018 | rs1150757 | MHC | CLIC1 |
| rs3101018 | rs1150757 | MHC | HSPA1L |
| rs3101018 | rs1150757 | MHC | ABHD16A |
| rs3101018 | rs1150757 | MHC | STK19 |
| rs3101018 | rs1150757 | MHC | HSPA1A |
| rs886582 | rs34548580 | PLD2 | SLC25A11 |
| rs886582 | rs34548580 | PLD2 | RNF167 |
| rs886582 | rs34548580 | PLD2 | CYB5D2 |
| rs886582 | rs34548580 | PLD2 | ZZEF1 |
| rs72787721 | rs17321999 | LBH | TCONS_00003622 |
| rs72787721 | rs17321999 | LBH | LCLAT1 |
| rs72787721 | rs17321999 | LBH | TCONS_00004194 |
| rs72787721 | rs17321999 | LBH | TCONS_00004193 |
| rs72787721 | rs17321999 | LBH | TCONS_00005128 |
| rs3024505 | rs3024505 | IL10 | IL19 |
| rs3024505 | rs3024505 | IL10 | IL20 |
| rs1453560 | rs12938617 | IKZF3 | STARD3 |
| rs1453560 | rs12938617 | IKZF3 | RP11-387H17.4 |
| rs1453560 | rs12938617 | IKZF3 | PSMD3 |
| rs1453560 | rs12938617 | IKZF3 | GSDMB |
| rs1453560 | rs12938617 | IKZF3 | PGAP3 |
| rs1453560 | rs12938617 | IKZF3 | ERBB2 |
| rs1453560 | rs12938617 | IKZF3 | IKZF3 |
| rs1453560 | rs12938617 | IKZF3 | ORMDL3 |
| rs1453560 | rs12938617 | IKZF3 | ORMDL3 |
| rs1453560 | rs12938617 | IKZF3 | LRRC3C |
| rs1453560 | rs2941509 | IKZF3 | STARD3 |
| rs1453560 | rs2941509 | IKZF3 | RP11-387H17.4 |
| rs1453560 | rs2941509 | IKZF3 | PSMD3 |
| rs1453560 | rs2941509 | IKZF3 | GSDMB |
| rs1453560 | rs2941509 | IKZF3 | PGAP3 |
| rs1453560 | rs2941509 | IKZF3 | ERBB2 |
| rs1453560 | rs2941509 | IKZF3 | IKZF3 |
| rs1453560 | rs2941509 | IKZF3 | ORMDL3 |
| rs1453560 | rs2941509 | IKZF3 | ORMDL3 |
| rs1453560 | rs2941509 | IKZF3 | LRRC3C |
| rs12046369 | rs1538633 | IL28RA | NIPAL3 |
| rs12046369 | rs1538633 | IL28RA | STPG1 |
| rs12046369 | rs1538633 | IL28RA | GRHL3 |
| rs12046369 | rs1538633 | IL28RA | RP11-10N16.3 |
| rs849333 | rs12531540 | JAZF1 | AC004549.6 |
| rs849333 | rs12531540 | JAZF1 | TAX1BP1 |
| rs849333 | rs12531540 | JAZF1 | HIBADH |
| rs849333 | rs849142 | JAZF1 | AC004549.6 |
| rs849333 | rs849142 | JAZF1 | TAX1BP1 |
| rs849333 | rs849142 | JAZF1 | HIBADH |
| rs7902904 | rs10761602 | ARID5B | RTKN2 |
| rs7902904 | rs10761602 | ARID5B | ARID5B |
| rs7902904 | rs4948496 | ARID5B | RTKN2 |
| rs7902904 | rs4948496 | ARID5B | ARID5B |
| rs2297548 | rs2297550 | IKBKE | DYRK3 |
| rs2297548 | rs2297550 | IKBKE | RASSF5 |
| rs2297548 | rs2297550 | IKBKE | RASSF5 |
| rs2297548 | rs2297550 | IKBKE | RASSF5 |
| rs2297548 | rs2297550 | IKBKE | RASSF5 |
| rs2297548 | rs2297550 | IKBKE | EIF2D |
| rs2297548 | rs2297550 | IKBKE | MAPKAPK2 |
| rs2297548 | rs2297550 | IKBKE | RP11-343H5.6 |
| 13:41635056 | rs17630801 | ELF1 | MTRF1 |
| 13:41635056 | rs17630801 | ELF1 | KBTBD7 |
| 13:41635056 | rs17630801 | ELF1 | ELF1 |
| 13:41635056 | rs17630801 | ELF1 | ELF1 |
| 13:41635056 | rs17630801 | ELF1 | NAA16 |
| 13:41635056 | rs7329174 | ELF1 | MTRF1 |
| 13:41635056 | rs7329174 | ELF1 | KBTBD7 |
| 13:41635056 | rs7329174 | ELF1 | ELF1 |
| 13:41635056 | rs7329174 | ELF1 | ELF1 |
| 13:41635056 | rs7329174 | ELF1 | NAA16 |
| rs653520 | rs77000060 | TNFAIP3 | TCONS_00012608 |
| rs653520 | rs77000060 | TNFAIP3 | RP11-240M16.1 |
| rs653520 | rs77000060 | TNFAIP3 | RP11-10J5.1 |
| rs653520 | rs77000060 | TNFAIP3 | TCONS_00012793 |
| rs653520 | rs77000060 | TNFAIP3 | TCONS_00012609 |
| rs653520 | rs77000060 | TNFAIP3 | TNFAIP3 |
| rs653520 | rs77000060 | TNFAIP3 | RP11-356I2.4 |
| rs653520 | rs2230926 | TNFAIP3 | TCONS_00012608 |
| rs653520 | rs2230926 | TNFAIP3 | RP11-240M16.1 |
| rs653520 | rs2230926 | TNFAIP3 | RP11-10J5.1 |
| rs653520 | rs2230926 | TNFAIP3 | TCONS_00012793 |
| rs653520 | rs2230926 | TNFAIP3 | TCONS_00012609 |
| rs653520 | rs2230926 | TNFAIP3 | TNFAIP3 |
| rs653520 | rs2230926 | TNFAIP3 | RP11-356I2.4 |
| rs3130288 | rs1150757 | MHC | PPT2 |
| rs3130288 | rs1150757 | MHC | PPT2-EGFL8 |
| rs3130288 | rs1150757 | MHC | AGPAT1 |
| rs3130288 | rs1150757 | MHC | AGPAT1 |
| rs3130288 | rs1150757 | MHC | RNF5 |
| rs3130288 | rs1150757 | MHC | PBX2 |
| rs3130288 | rs1150757 | MHC | GPSM3 |
| rs3130288 | rs1150757 | MHC | PRRT1 |
| rs4090311 | rs1538633 | IL28RA | NIPAL3 |
| rs4090311 | rs1538633 | IL28RA | STPG1 |
| rs4090311 | rs1538633 | IL28RA | STPG1 |
| rs4090311 | rs1538633 | IL28RA | STPG1 |
| rs4090311 | rs1538633 | IL28RA | STPG1 |
| rs4090311 | rs4649203 | IL28RA | NIPAL3 |
| rs4090311 | rs4649203 | IL28RA | STPG1 |
| rs4090311 | rs4649203 | IL28RA | STPG1 |
| rs4090311 | rs4649203 | IL28RA | STPG1 |
| rs4090311 | rs4649203 | IL28RA | STPG1 |
| rs60568231 | rs12418883 | IRF7 | DEAF1 |
| rs60568231 | rs12418883 | IRF7 | DEAF1 |
| rs60568231 | rs12418883 | IRF7 | HRAS |
| rs60568231 | rs12418883 | IRF7 | HRAS |
| rs60568231 | rs12418883 | IRF7 | AP006621.9 |
| rs60568231 | rs12418883 | IRF7 | AP006621.9 |
| rs60568231 | rs12418883 | IRF7 | RNH1 |
| rs60568231 | rs12418883 | IRF7 | EPS8L2 |
| rs60568231 | rs12418883 | IRF7 | EPS8L2 |
| rs60568231 | rs12418883 | IRF7 | EPS8L2 |
| rs60568231 | rs12418883 | IRF7 | PSMD13 |
| rs60568231 | rs12418883 | IRF7 | LRRC56 |
| rs60568231 | rs12418883 | IRF7 | LRRC56 |
| rs60568231 | rs12418883 | IRF7 | PIDD |
| rs60568231 | rs12418883 | IRF7 | TMEM80 |
| rs60568231 | rs12418883 | IRF7 | CEND1 |
| rs60568231 | rs12418883 | IRF7 | RASSF7 |
| rs60568231 | rs12418883 | IRF7 | SIRT3 |
| rs60568231 | rs12418883 | IRF7 | C11orf35 |
| rs60568231 | rs12418883 | IRF7 | IRF7 |
| rs60568231 | rs1061502 | IRF7 | DEAF1 |
| rs60568231 | rs1061502 | IRF7 | DEAF1 |
| rs60568231 | rs1061502 | IRF7 | HRAS |
| rs60568231 | rs1061502 | IRF7 | HRAS |
| rs60568231 | rs1061502 | IRF7 | AP006621.9 |
| rs60568231 | rs1061502 | IRF7 | AP006621.9 |
| rs60568231 | rs1061502 | IRF7 | RNH1 |
| rs60568231 | rs1061502 | IRF7 | EPS8L2 |
| rs60568231 | rs1061502 | IRF7 | EPS8L2 |
| rs60568231 | rs1061502 | IRF7 | EPS8L2 |
| rs60568231 | rs1061502 | IRF7 | PSMD13 |
| rs60568231 | rs1061502 | IRF7 | LRRC56 |
| rs60568231 | rs1061502 | IRF7 | LRRC56 |
| rs60568231 | rs1061502 | IRF7 | PIDD |
| rs60568231 | rs1061502 | IRF7 | TMEM80 |
| rs60568231 | rs1061502 | IRF7 | CEND1 |
| rs60568231 | rs1061502 | IRF7 | RASSF7 |
| rs60568231 | rs1061502 | IRF7 | SIRT3 |
| rs60568231 | rs1061502 | IRF7 | C11orf35 |
| rs60568231 | rs1061502 | IRF7 | IRF7 |
| rs374974295 | rs4388254 | TCF7 | TCF7 |
| rs374974295 | rs4388254 | TCF7 | TCF7 |
| rs1887428 | rs1887428 | JAK2 | ERMP1 |
| rs1887428 | rs1887428 | JAK2 | CD274 |
| rs1887428 | rs1887428 | JAK2 | RCL1 |
| rs1887428 | rs1887428 | JAK2 | AK3 |
| rs1887428 | rs1887428 | JAK2 | CDC37L1 |
| rs1887428 | rs1887428 | JAK2 | RP11-6J24.6 |
| rs1887428 | rs1887428 | JAK2 | PPAPDC2 |
| rs7202408 | rs9652601 | CIITA | RP11-485G7.5 |
| rs7202408 | rs9652601 | CIITA | RMI2 |
| rs11606612 | rs3794060 | DHCR7 | TCONS_00019374 |
| rs11606612 | rs3794060 | DHCR7 | RP11-660L16.2 |
| rs11606612 | rs3794060 | DHCR7 | DHCR7 |
| rs11606612 | rs7118246 | DHCR7 | TCONS_00019374 |
| rs11606612 | rs7118246 | DHCR7 | RP11-660L16.2 |
| rs11606612 | rs7118246 | DHCR7 | DHCR7 |

### Example II

### Methods for Diagnosis of Inflammatory Disorders and Screening Assays to Identify Therapeutic Agents Useful for the Treatment of the Same

The information herein above can be applied clinically to patients for diagnosing the presence of, or an increased susceptibility for developing an inflammatory disorder, and for therapeutic intervention. A preferred embodiment of the invention comprises clinical application of the information described herein to a patient. Diagnostic compositions, including microarrays, and methods can be designed to identify the gene targets and appropriate therapeutic as described herein in nucleic acids from a patient to assess susceptibility for developing inflammatory disorders, including SLE. This can occur after a patient arrives in the clinic; the patient has blood drawn, and using the diagnostic methods described herein, a clinician can detect the SNPs in the chromosomal regions described herein. The information obtained from the patient sample, which can optionally be amplified prior to assessment, will be used to diagnose a patient with an increased or decreased susceptibility for developing an inflammatory disorder. Kits for performing the diagnostic method of the invention are also provided herein. Such kits comprise a microarray comprising at least one of the SNPs provided herein in and the necessary reagents for assessing the patient samples as described above. Capture C genes associated with SLE present in TFH are listed in Table 1 Agents targeting these genes and gene products are also provided in Table 2. In certain embodiments, inhibitors that target HIPK1 and MINK1 are employed. These include, but are not limited to ‘A64’—a high affinity HIPK2 inhibitor that has a low affinity for HIPK1—and the MAP4K2 inhibitor PF06260933, which also inhibits MINK1.

TABLE 2
Genes involved in inflammatory disorders such as SLE and drugs
which modulate the same

| | |
|---|---|
| CAMLG calcium modulating ligand | alemtuzumab/cyclosporin A, cyclosporine |
| | A/tacrolimus, sirolimus/tacrolimus, methotrexate |
| CD274 CD274 molecule | atezolizumab, durvalumab, avelumab, anti PD-L1 |
| | antibody, FAZ053, CX-072, LY3300054, CA-170, |
| | KN035, CK-301, CS1001, MSB0011359C, BCD- |
| | 135, KN046, FS118, HLX20, atezolizumab/ |
| | bevacizumab, 89Zr-avelumab, LY3415244, |
| | INBRX 105, atezolizumab/paclitaxel, TG-1501, |
| | LY3434172, GEN1046, TQB2450 |
| CD44 CD44 molecule | A6 peptide, anti-CD44v7 antibody, AMC303 |
| (Indian blood group) | |
| CD80 CD80 molecule | abatacept, galiximab, belatacept, |
| | abatacept/methotrexate |
| CHRNE cholinergic receptor | ABT-089, isoflurane, mecamylamine, |
| nicotinic epsilon subunit | succinylcholine, rocuronium, doxacurium, |
| | amobarbital, mivacurium, pipecuronium, |
| | rapacuronium, metocurine, atracurium, |
| | cisatracurium, acetylcholine, nicotine, D- |
| | tubocurarine, arecoline, enflurane, pancuronium, |
| | vecuronium |
| CSK C-terminal Src kinase | bosutinib |
| ERBB2 erb-b2 receptor tyrosine kinase 2 | trastuzumab, BMS-599626, varlitinib, tesevatinib, |
| | CP-724, 714, BMS-690514, dacomitinib, afatinib, |
| | pertuzumab, ertumaxomab, MP 412, sapitinib, |
| | mubritinib, trastuzumab emtansine, CUDC 101, |
| | lapatinib/pazopanib, JNJ-26483327, |
| | lapatinib/letrozole, cyclophosphamide/ |
| | docetaxel/doxorubicin/trastuzumab, |
| | cyclophosphamide/doxorubicin/paclitaxel/ |
| | trastuzumab, paclitaxel/trastuzumab, |
| | capecitabine/lapatinib, docetaxel/ |
| | pertuzumab/trastuzumab, cyclophosphamide/ |
| | docetaxel/epirubicin/5-fluorouracil/trastuzumab, |
| | docetaxel/trastuzumab, paclitaxel/ |
| | pertuzumab/trastuzumab, trastuzumab/vinorelbine, |
| | capecitabine/trastuzumab, lapatinib/paclitaxel, |
| | pertuzumab/trastuzumab, cyclophosphamide/ |
| | doxorubicin/paclitaxel/pertuzumab/trastuzumab, |
| | MGAH22, osimertinib, 89Zr-trastuzumab, |
| | tucatinib, poziotinib, [68Ga]ABY-025, pyrotinib, |
| | selatinib, MM-111, lapatinib/trastuzumab, MM- |
| | 302, docetaxel/lapatinib, cyclophosphamide/ |
| | epirubicin/5-fluorouracil/paclitaxel/trastuzumab, |
| | cyclophosphamide/docetaxel/doxorubicin/ |
| | pertuzumab/trastuzumab, cyclophosphamide/ |
| | docetaxel/trastuzumab |
| FDFT1 farnesyl-diphosphate | lapaquistat, zoledronic acid |
| farnesyltransferase 1 | |
| GSK3B glycogen synthase kinase 3 beta | enzastaurin, glycogen synthase kinase-3beta |
| | inhibitor |
| HRAS HRas proto-oncogene, GTPase | ISIS 2503 |
| IGF1 insulin like growth factor 1 | MEDI-573, BI 836845 |
| LTA lymphotoxin alpha | etanercept, etanercept/methotrexate |
| MAPKAPK2 MAPK activated | cdc25C phosphatase (211-221) |
| protein kinase 2 | |
| PDE4A phosphodiesterase 4A | enprofylline, dyphylline, caffeine/ergotamine, |
| | arofylline, tetomilast, L 869298, ibudilast, |
| | apremilast, aspirin/dipyridamole/telmisartan, |
| | drotaverin, anagrelide, cilomilast, milrinone, |
| | dipyridamole, L-826,141, aspirin/dipyridamole, |
| | amrinone, ketotifen, roflumilast, theophylline, |
| | pentoxifylline, caffeine |
| PTPRC protein tyrosine phosphatase | 111 In-BC8, At 211 MAbBC8-B10 |
| receptor type C | |
| TNF tumor necrosis factor | adalimumab, etanercept, infliximab, certolizumab, |
| | golimumab, tumor necrosis factor receptor |
| | antagonist, afelimomab, infliximab/methotrexate, |
| | dexamethasone/thalidomide, dexamethasone/ |
| | pomalidomide, cyclophosphamide/dexamethasone/ |
| | thalidomide, golimumab/methotrexate, |
| | bortezomib/dexamethasone/thalidomide, |
| | rituximab/thalidomide, bortezomib/thalidomide, |
| | prednisone/thalidomide, adalimumab/methotrexate, |
| | etanercept/methotrexate, pomalidomide, |
| | bortezomib/dexamethasone/pomalidomide, |
| | thalidomide, infliximab/methylprednisolone, |
| | infliximab/prednisone, certolizumab/methotrexate, |
| | bevacizumab/docetaxel/prednisone/thalidomide/ |
| | zoledronic acid |

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for alleviating inflammatory disease symptoms in a patient in need thereof, comprising;
a) identifying in a nucleic acid containing biological sample from said patient, an inflammatory disease GWAS causal variant in a gene which is indicative of the presence of, or increased risk for, inflammatory disease;
b) treating said patient with an effective amount of at least one agent which targets said gene harboring said causal variant, thereby alleviating inflammatory disease symptoms.

2. The method of claim 1, wherein said inflammatory disease is selected from systemic lupus erythematosus (SLE), arthritis, rheumatoid arthritis, psoriatic arthritis, juvenile idiopathic arthritis, multiple sclerosis, myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, scleroderma, systemic sclerosis, or allergic asthma.

3. The method of claim 1, wherein said inflammatory disease is SLE.

4. The method of claim 1, wherein proximal and sentinel SNPs, inferred GWAS causal variant and genes implicated in 3D epigenomics assays are listed in Table 1.

5. The method of claim 4, wherein implicated genes and suitable therapeutic are listed in Table 2.

6. The method of claim 4, wherein said gene is MINK1 and the agent is a MAP3/4K antagonist.

7. A method for identifying an agent useful for the treatment of inflammatory disease for use in the method of claim 1, comprising;
a) providing a cell harboring at least one gene comprising an informative SNP for inflammatory disease in a cell type of interest and a cell which lacks said informative SNP;
b) incubating said cells in the presence with an agent; and
c) identifying agents which alter the function of said gene in cells harboring said SNP relative to those lacking said SNP.

8. The method of claim 1, wherein said cells are selected from tonsil follicular T helper cells, naïve CD4+ T cells, naïve CD8+ T cells, memory CD4+ T cells, memory CD8+ T cells, cytotoxic T lymphocytes, naïve B cells, germinal center B cells, Th1 cells, Th2 cells, Th17 cells, NK cells, dendritic cells, monocytes

9. The method of claim 7, wherein said gene and said agent is provided in Table 2.

10. A method for treatment of SLE comprising administration of an effective amount of
i) a MAP3/4K antagonist; or,
ii) a pharmacological inhibitor of HIPK1; said treatment alleviating SLE symptoms.

11. The method of claim 10, wherein said agent is PF06260933.

12. The method of claim 10, further comprising administration of a steroid.

13. (canceled)

14. A transgenic mouse which is a knockout mouse for HIPK1 or MINK1 for use in the method of claim 7.

15. The method of claim 7, wherein said agent is screened in a transgenic mouse comprising a nucleic acid harboring a loss of function mutation in HIPK1 or MINK1.

16. An EBV transformed cell line harboring at least one gene comprising an informative SNP for an inflammatory disease in a cell type of interest as claimed in claim 1, wherein said gene is encoded by
i) a nucleic acid comprising a mutation in a HIPK1 gene isolated from a patient having altered immune function, or
ii) a mutation in a nucleic acid comprising a MINK1 gene isolated from a patient having altered immune function, or
iii) a nucleic acid harboring a mutation in a gene listed in Table 1.

17. (canceled)

18. (canceled)

19. (canceled)


SEQUENCE LISTING (12 sequences)

SEQ ID NO: 1 - DNA, 21 bp, Artificial Sequence
  BLC6-Forward
ctctgtggttgtgggcaaggc

SEQ ID NO: 2 - DNA, 21 bp, Artificial Sequence
  BLC6-Reverse
caggtggcgaatcaggacagg

SEQ ID NO: 3 - DNA, 24 bp, Artificial Sequence
  CXCR5-Forward
gtccctggtgatggaaactcaggc

SEQ ID NO: 4 - DNA, 22 bp, Artificial Sequence
  CXCR5-Reverse
gcagtggcctcccttacacagg

SEQ ID NO: 5 - DNA, 23 bp, Artificial Sequence
  IKZF1-Forward
ccttctccatgcccaggtgactc

SEQ ID NO: 6 - DNA, 23 bp, Artificial Sequence
  IKZF1-Reverse
ggcctcagctaggcaaaccagag

SEQ ID NO: 7 - DNA, 20 bp, Artificial Sequence
  HIPK-1 Forward
cagtcaggagttctcacgca

SEQ ID NO: 8 - DNA, 20 bp, Artificial Sequence
  HIPK-1 Reverse
tggctacttgagggtggaga

SEQ ID NO: 9 - DNA, 20 bp, Artificial Sequence
  B2M Forward
gccgtgtgaaccatgtgact

SEQ ID NO: 10 - DNA, 20 bp, Artificial Sequence
  B2M Reverse
catccaatccaaatgcggca

SEQ ID NO: 11 - DNA, 22 bp, Artificial Sequence
  hu 18S Forward
cctttaacgaggatccattgga

SEQ ID NO: 12 - DNA, 22 bp, Artificial Sequence
  hu 18S Reverse
cgctattggagctggaattacc